(19) United States
(12) Patent Application Publication
(10) Pub. No.: US 2024/0132921 A1
(43) Pub. Date: Apr. 25, 2024

(54) MICROBIAL PRODUCTION OF TYROSOL AND SALIDROSIDE

(71) Applicant: SILICOLIFE LDA., Braga (PT)

(72) Inventors: Simão Pedro DE PINHO SOARES, Braga (PT); Joana Margarida SILVA GOMES, Braga (PT); Cristiana DA SILVA FARIA, Braga (PT); Isabel Cristina DE ALMEIDA PEREIRA DA ROCHA, Lisbon (PT); Paulo Ricardo CARVALHO VILAÇA, Braga (PT)

(73) Assignee: SILICOLIFE LDA., Braga (PT)

(21) Appl. No.: 18/263,894

(22) PCT Filed: Feb. 8, 2022
(86) PCT No.: PCT/EP2022/053036

§ 371 Date: Aug. 2, 2023

(30) Foreign Application Priority Data
Feb. 8, 2021 (EP) 21155780.6
Jul. 13, 2021 (PT) 117340 K
Sep. 13, 2021 (EP) 21196276.6

(51) Int. Cl. C12P 7/22; C12N 9/02; C12N 9/10; C12N 9/88; C12N 15/70; C12P 19/44

(52) U.S. Cl. CPC C12P 7/22; C12N 9/001; C12N 9/1085; C12N 9/88; C12N 15/70; C12P 19/44; C12Y 103/01012; C12Y 205/01054; C12Y 401/01043

(57) ABSTRACT

The invention relates to a method for production of tyrosol, wherein a transgenic bacterial cell that heterologously expresses phenylpyruvate decarboxylase and that overexpresses phospho-2-dehydro-3-deoxyheptonate and prephenate dehydrogenase, and wherein pheAL and feaB are both inactivated or removed, is grown in a medium comprising a metabolic precursor of phosphoenolpyruvate (PEP) and erythrose 4-phosphate (E4P), particularly glucose, and optionally, phenylalanine as a supplement; and tyrosol is extracted from said medium. The invention also relates to a method for production of salidroside, wherein the transgenic cell additionally heterologously expresses uridine diphosphate dependent glycosyltransferase (UGT85A1, EC:2.4.1.)

This application claims the benefit of European Patent Applications EP21155780.6, filed 8 Feb. 2021, and EP21196276.6 filed 13 Sep. 2021 and of the Portuguese Patent Application 20211000027222, filed 13 Jul. 2021, all of which are incorporated herein by reference.

The invention relates to a method for production of tyrosol, wherein a transgenic bacterial cell that heterologously expresses phenylpyruvate decarboxylase and that overexpresses phospho-2-dehydro-3-deoxyheptonate and prephenate dehydrogenase, and wherein pheAL and feaB are both inactivated or removed, is grown in a medium comprising a metabolic precursor of phosphoenolpyruvate (PEP) and erythrose 4-phosphate (E4P), particularly glucose, and optionally, phenylalanine as a supplement; and tyrosol is extracted from said medium. The invention also relates to a method for production of salidroside, wherein the transgenic cell additionally heterologously expresses uridine diphosphate dependent glycosyltransferase (UGT85A1, EC:2.4.1.).

### DESCRIPTION

Tyrosol is a phenolic compound of great industrial value and is marketed as a fine chemical.

Salidroside is a glucoside of tyrosol and has been studied as one of the potential compounds responsible for its putative antidepressant and anxiolytic actions.

Tyrosol concentration in plants is usually low, which leads to low commercial product yields and high production costs. Moreover, the natural extraction process for obtaining high purity tyrosol from plants is complex, which also makes the yield relatively low. Despite its natural abundance, because the cost of its extraction from natural sources is very high, tyrosol is also produced via chemical synthesis methods for industrial purposes, but these methods leave much room for improvement from a commercial point of view.

### Definitions

Transgenic cell as referred to in the current context means that the cell comprises at least one gene derived from a different organism than the host cell (referred to in the current specification as the transgene). This gene is introduced into the transgenic host cell via molecular biology methods.

Heterologous expression or heterologously expresses in relation to a certain gene as referred to in the current specification means that the gene is derived from a source other than the host species in which it is said to be heterologously expressed.

Overexpressing or overexpression in relation to a certain gene as referred to in the current specification means: addition of a functional (transgene or autologous) version of said gene, and/or addition of a promoter sequence controlling the autologous (native) version of said gene, leading to a significantly higher expression of the gene's biological activity relative to the wild-type (bacterial) cell. Significantly higher expression of the gene's biological activity means that there are at least 1.5-fold, particularly at least two-fold, the number of mRNA molecules inside the bacterial cell, compared to the wild-type bacterial cell. The overexpressed gene may also comprise mutations (substitutions, deletions and/or insertions) compared to the wild type nucleic acid and amino acid sequence. The mutations may increase the enzymatic efficacy, optimize the expression rate or change the enzymatic specificity.

Inactivation or knock-out in relation to a certain gene as referred to in the current specification means that the expression of that gene is significantly reduced, particularly by at least 30-fold, more particularly by at least 100-fold, compared to the wild-type bacterial cell or there is no gene expression of that gene.

Recombinant gene expression in relation to a certain gene as referred to in the current specification means: The recombinant gene is inserted into the host cell by molecular biology methods. The recombinant gene may originate from the same organism as the host cell, or from a different organism.

Supplement refers to amounts of a compound which are not the main carbon source for the bacterial cell, but are given in sufficient amounts that the cell's metabolism can compensate for auxotrophy of the compound. Phenylalanine is needed to cover the auxotrophy of pheAL deletion strains. The inventors used M9Y as it has yeast extract as a source of phenylalanine. Supplementation is needed either with yeast extract or pure phenylalanine.

### DETAILED DESCRIPTION OF THE INVENTION

A first aspect of the invention relates to a method for production of tyrosol, wherein a transgenic bacterial cell that heterologously expresses the following enzyme:
a. phenylpyruvate decarboxylase (ARO10, EC:4.1.1.80) and that overexpresses each of the following enzymes:
b. phospho-2-dehydro-3-deoxyheptonate aldolase (aroF, EC:2.5.1.54)
c. prephenate dehydrogenase (tyrA, EC:5.4.99.5 and EC:1.3.1.12) and wherein each of the following genes is inactivated or removed (not present, not expressed):
i. pheAL (Bifunctional chorismate mutase/prephenate dehydratase (UniProtKB—P0A9J8; EC:5.4.99.5)
ii. feaB (Phenylacetaldehyde dehydrogenase, UniProtKB—P80668; EC:1.2.1.39) is grown in a medium comprising
   a metabolic precursor of phosphoenolpyruvate (PEP) and erythrose 4-phosphate (E4P), particularly glucose, and
   optionally, phenylalanine as a supplement;
and tyrosol is extracted from said medium.

In certain embodiments, the transgenic bacterial cell is of the genus Escherichia, In certain embodiments, the transgenic bacterial cell is of the species E. coli. In certain embodiments, the transgenic bacterial cell is of the strain E. coli BL21.

In certain embodiments, the gene encoding the phenylpyruvate decarboxylase originates from yeast. In certain embodiments, the gene encoding the phenylpyruvate decarboxylase originates from S. cerevisiae.

A second aspect of the invention relates to a method for production of salidroside, wherein
the transgenic cell as specified in any of the preceding embodiments additionally heterologously expresses uridine diphosphate dependent glycosyltransferase (UGT85A1, EC:2.4.1.), and
the cell is grown in a medium comprising
   a metabolic precursor of phosphoenolpyruvate (PEP) and erythrose 4-phosphate (E4P), particularly glucose, and
   optionally, phenylalanine as a supplement;
and salidroside is extracted from said medium.

A third aspect of the invention relates to a method for production of hydroxytyrosol, wherein a transgenic bacterial cell that heterologously expresses the following enzyme:
a. phenylpyruvate decarboxylase (ARO10, EC:4.1.1.80) and that overexpresses each of the following enzymes:
b. phospho-2-dehydro-3-deoxyheptonate aldolase (aroF, EC:2.5.1.54)
c. prephenate dehydrogenase (tyrA, EC:5.4.99.5 and EC:1.3.1.12)
d. 4-hydroxyphenylacetate 3-monooxygenase (hpaBC*, EC:1.14.14.9) is grown in a medium comprising
   a metabolic precursor of phosphoenolpyruvate (PEP) and erythrose 4-phosphate (E4P), particularly glucose, and
   optionally, phenylalanine as a supplement;
and hydroxytyrosol is extracted from said medium.

An alternative of the third aspect of the invention relates to a method for production of hydroxytyrosol, wherein a transgenic bacterial cell that recombinantly expresses each of the following enzymes:
a. phenylpyruvate decarboxylase (ARO10, EC:4.1.1.80)
b. phospho-2-dehydro-3-deoxyheptonate aldolase (aroF, EC:2.5.1.54)
c. prephenate dehydrogenase (tyrA, EC:5.4.99.5 and EC:1.3.1.12)
d. 4-hydroxyphenylacetate 3-monooxygenase (hpaBC*, EC:1.14.14.9) is grown in a medium comprising
   a metabolic precursor of phosphoenolpyruvate (PEP) and erythrose 4-phosphate (E4P), particularly glucose, and
   optionally, phenylalanine as a supplement;
and hydroxytyrosol is extracted from said medium.

In certain embodiments, the gene encoding the 4-hydroxyphenylacetate 3-monooxygenase originates from Escherichia. In certain embodiments, the gene encoding the 4-hydroxyphenylacetate 3-monooxygenase originates from E. coli.

In certain embodiments, the gene encoding the 4-hydroxyphenylacetate 3-monooxygenase comprises amino acid substitutions S₂₁₀T, A₂₁₁L and Q₂₁₂E.

In certain embodiments of the third aspect, the medium comprises 5-10 g/L Na₂HPO₄·2H₂O, 2-4 g/L KH₂PO₄, 0.25-1 g/L NaCl, 0.5-1.5 g/L NH₄Cl, 1-3% (w/v) glucose, 0.01-0.05% (w/v) yeast extract, 3-7 mM MgSat, 0.005-0.02 g/L CaCl₂), 0.5-2.0 g/L ascorbic acid, and antibiotics.

In certain embodiments of the third aspect, dodecanol is added to the medium. In certain embodiments of the third aspect, ˜25% dodecanol (v/v) is added to the medium. As dodecanol is immiscible with water it builds a second layer on top of the culture medium.

In certain embodiments of the third aspect, the cells are grown with >2% (v/v) of 02. In certain embodiments of the third aspect, the cells are grown with 2-4% (v/v) of 02.

In certain embodiments, the gene encoding uridine diphosphate dependent glycosyltransferase originates from a plant. In certain embodiments, the gene encoding uridine diphosphate dependent glycosyltransferase originates from Arabidopsis. In certain embodiments, the gene encoding uridine diphosphate dependent glycosyltransferase originates from A. thaliana.

In certain embodiments, the transgenic bacterial cell does not overexpress any of the following proteins:
alcohol dehydrogenase, (UniProtKB—P39451; EC:1.1.1.1),
DNA-binding transcriptional regulatory protein (tyrR NCBI GenPept: NP_415839.1),
and tyrosine aminotransferase, (UniProtKB—P04693, EC:2.6.1.57).

In certain embodiments, the only transgenes of the transgenic bacterial cell are the ones mentioned above.

In certain embodiments, the overexpressed genes and the transgenes are introduced into the transgenic bacterial cell via one or several plasmid vector(s), particularly wherein
phenylpyruvate decarboxylase, phospho-2-dehydro-3-deoxyheptonate aldolase and prephenate dehydrogenase are encoded by a medium-copy plasmid vector, and/or
uridine diphosphate dependent glycosyltransferase is encoded by a low-copy plasmid vector, and/or
4-hydroxyphenylacetate 3-monooxygenase is encoded by a low-copy plasmid vector.

In certain embodiments, said transgenic bacterial cell comprises one or more plasmids encoding said heterologously expressed or overexpressed enzymes under control of a promoter sequence operable in said cell, particularly a T7 promoter (SEQ ID NO. 31), a lac promoter (SEQ ID NO. 32), a tac promoter (SEQ ID NO. 33) or a trc promoter (SEQ ID NO. 34), more particularly wherein
the gene encoding uridine diphosphate dependent glycosyltransferase is under control of a trc promoter, and/or
the gene encoding phenylpyruvate decarboxylase is under control of a T7 promoter, and/or
the gene encoding phospho-2-dehydro-3-deoxyheptonate aldolase is under control of a T7 promoter, and/or
the gene encoding prephenate dehydrogenase is under control of a T7 promoter; and/or
the gene encoding 4-hydroxyphenylacetate 3-monooxygenase is under control of a T7 promoter.

In certain embodiments, the expression of said heterologous and/or overexpressed genes is induced by adding isopropyl-β-d-thiogalactopyranoside (IPTG), particularly at a concentration of ˜0.1 mM IPTG for 96 h.

In certain embodiments, said medium comprises 10 to 50 g/L of glucose, particularly 15 to 30 g/L of glucose.

In certain embodiments, the transgenes are codon-optimized for expression in said transgenic bacterial cell.

In certain embodiments, the medium comprises 5-10 g/L Na₂HPO₄·2H₂O, 2-4 g/L KH₂PO₄, 0.25-1 g/L NaCl, 0.5-1.5 g/L NH₄Cl, 1-3% (w/v) glucose, 0.01-0.05% (w/v) yeast extract, 3-7 mM MgSO₄, 0.005-0.02 g/L CaCl₂) and antibiotics, particularly the antibiotics are 50-200 μg/mL ampicillin, 10-50 μg/mL kanamycin and 25-45 μg/mL chloramphenicol.

In certain embodiments, the cell is grown at 22° C. to 30° C., particularly at ˜30° C.

In certain embodiments, the protein phenylpyruvate decarboxylase has at least 60%, 65%, 70%, 75%, 80%, 85%, 90%, 95% or >95% sequence identity with SEQ ID NO 1 and has a catalytic activity of at least 75% of the activity of SEQ ID NO 1. In certain embodiments, the protein phospho-2-dehydro-3-deoxyheptonate aldolase has at least 60%, 65%, 70%, 75%, 80%, 85%, 90%, 95% or >95% sequence identity with SEQ ID NO 2 and has a catalytic activity of at least 75% of the activity of SEQ ID NO 2. In certain embodiments, the protein prephenate dehydrogenase has at least 60%, 65%, 70%, 75%, 80%, 85%, 90%, 95% or >95% sequence identity with SEQ ID NO 3 and has a catalytic activity of at least 75% of the activity of SEQ ID NO 3. In certain embodiments, the protein uridine diphosphate dependent glycosyltransferase has at least 60%, 65%, 70%, 75%, 80%, 85%, 90%, 95% or >95% sequence identity with SEQ ID NO 4 and has a catalytic activity of at least 75% of the activity of SEQ ID NO 4. In certain embodiments, the protein 4-hydroxyphenylacetate 3-monooxygenase has at least 60%, 65%, 70%, 75%, 80%, 85%, 90%, 95% or >95% sequence identity with SEQ ID NO 035 and has a catalytic activity of at least 75% of the activity of SEQ ID NO 035.

A fourth aspect of the invention relates to a transgenic cell as specified in any one of the above stated embodiments.

An alternative of the fourth aspect relates to a transgenic cell that heterologously expresses the following enzyme:
a. phenylpyruvate decarboxylase (ARO10, EC:4.1.1.80) and that overexpresses each of the following enzymes:
b. phospho-2-dehydro-3-deoxyheptonate aldolase (aroF, EC:2.5.1.54)
c. prephenate dehydrogenase (tyrA, EC:5.4.99.5 and EC:1.3.1.12)
and wherein each of the following genes is inactivated or removed (not present, not expressed):
i. pheAL (Bifunctional chorismate mutase/prephenate dehydratase (UniProtKB—P0A9J8; EC:5.4.99.5)
ii. feaB (Phenylacetaldehyde dehydrogenase, UniProtKB—P80668; EC:1.2.1.39).

Another alternative of the fourth aspect relates to a transgenic cell that heterologously expresses each of the following enzymes:
a. phenylpyruvate decarboxylase (ARO10, EC:4.1.1.80);
b. uridine diphosphate dependent glycosyltransferase (UGT85A1, EC:2.4.1.); and that overexpresses each of the following enzymes:
c. phospho-2-dehydro-3-deoxyheptonate aldolase (aroF, EC:2.5.1.54)
d. prephenate dehydrogenase (tyrA, EC:5.4.99.5 and EC:1.3.1.12) and wherein each of the following genes is inactivated or removed (not present, not expressed):
i. pheAL (Bifunctional chorismate mutase/prephenate dehydratase (UniProtKB—P0A9J8; EC:5.4.99.5)
ii. feaB (Phenylacetaldehyde dehydrogenase, UniProtKB—P80668; EC:1.2.1.39).

Another alternative of the fourth aspect relates to a transgenic cell that heterologously expresses the following enzyme:
a. phenylpyruvate decarboxylase (ARO10, EC:4.1.1.80) and that overexpresses each of the following enzymes:
b. phospho-2-dehydro-3-deoxyheptonate aldolase (aroF, EC:2.5.1.54)
c. prephenate dehydrogenase (tyrA, EC:5.4.99.5 and EC:1.3.1.12);
d. 4-hydroxyphenylacetate 3-monooxygenase (hpaBC*, EC:1.14.14.9).

In certain embodiments of the fourth aspect, the transgenic bacterial cell is of the genus Escherichia, particularly wherein the transgenic bacterial cell is of the species E. coli, more particularly wherein the transgenic bacterial cell is of the strain E. coli BL21.

In certain embodiments of the fourth aspect, the gene encoding the phenylpyruvate decarboxylase originates from yeast, particularly from S. cerevisiae.

In certain embodiments of the fourth aspect, the gene encoding the 4-hydroxyphenylacetate 3-monooxygenase originates from Escherichia, particularly from E. coli.

In certain embodiments of the fourth aspect, the gene encoding the 4-hydroxyphenylacetate 3-monooxygenase comprises amino acid substitutions S₂₁₀T, A₂₁₁L and Q₂₁₂E.

In certain embodiments of the fourth aspect, the gene encoding uridine diphosphate dependent glycosyltransferase originates from a plant, particularly from Arabidopsis, more particularly from A. thaliana.

In certain embodiments of the fourth aspect, the transgenic bacterial cell does not overexpress any of the following proteins:
alcohol dehydrogenase, (UniProtKB—P39451; EC:1.1.1.1),
DNA-binding transcriptional regulatory protein (tyrR NCBI GenPept: NP_415839.1),
and tyrosine aminotransferase, (UniProtKB—P04693, EC:2.6.1.57).

In certain embodiments of the fourth aspect, the only transgenes of the transgenic bacterial cell are the ones mentioned above.

In certain embodiments of the fourth aspect, the overexpressed genes and the transgenes are introduced into the transgenic bacterial cell via one or several plasmid vector(s), particularly wherein
phenylpyruvate decarboxylase, phospho-2-dehydro-3-deoxyheptonate aldolase and prephenate dehydrogenase are encoded by a medium-copy plasmid vector, and/or
uridine diphosphate dependent glycosyltransferase is encoded by a low-copy plasmid vector, and/or
4-hydroxyphenylacetate 3-monooxygenase is encoded by a low-copy plasmid vector.

In certain embodiments of the fourth aspect, said transgenic bacterial cell comprises one or more plasmids encoding said heterologously expressed or overexpressed enzymes under control of a promoter sequence operable in said cell, particularly a T7 promoter (SEQ ID NO. 31), a lac promoter (SEQ ID NO. 32), a tac promoter (SEQ ID NO. 33) or a trc promoter (SEQ ID NO. 34), more particularly wherein
the gene encoding uridine diphosphate dependent glycosyltransferase is under control of a trc promoter, and/or
the gene encoding phenylpyruvate decarboxylase is under control of a T7 promoter, and/or
the gene encoding phospho-2-dehydro-3-deoxyheptonate aldolase is under control of a T7 promoter, and/or
the gene encoding prephenate dehydrogenase is under control of a T7 promoter; and/or
the gene encoding 4-hydroxyphenylacetate 3-monooxygenase is under control of a T7 promoter.

In certain embodiments of the fourth aspect, the protein phenylpyruvate decarboxylase has at least 60%, 65%, 70%, 75%, 80%, 85%, 90%, 95% or >95% sequence identity with SEQ ID NO 1 and has a catalytic activity of at least 75% of the activity of SEQ ID NO 1. In certain embodiments of the fourth aspect, the protein phospho-2-dehydro-3-deoxyheptonate aldolase has at least 60%, 65%, 70%, 75%, 80%, 85%, 90%, 95% or >95% sequence identity with SEQ ID NO 2 and has a catalytic activity of at least 75% of the activity of SEQ ID NO 2. In certain embodiments of the fourth aspect, the protein prephenate dehydrogenase has at least 60%, 65%, 70%, 75%, 80%, 85%, 90%, 95% or >95% sequence identity with SEQ ID NO 3 and has a catalytic activity of at least 75% of the activity of SEQ ID NO 3. In certain embodiments of the fourth aspect, the protein uridine diphosphate dependent glycosyltransferase has at least 60%, 65%, 70%, 75%, 80%, 85%, 90%, 95% or >95% sequence identity with SEQ ID NO 4 and has a catalytic activity of at least 75% of the activity of SEQ ID NO 4. In certain embodiments of the fourth aspect, the protein 4-hydroxyphenylacetate 3-monooxygenase has at least 60%, 65%, 70%, 75%, 80%, 85%, 90%, 95% or >95% sequence identity with SEQ ID NO 035 and has a catalytic activity of at least 75% of the activity of SEQ ID NO 035.

The present specification also comprises the following items.

Items
1. A method for production of hydroxytyrosol, wherein a transgenic bacterial cell that heterologously expresses the following enzyme:
   a. phenylpyruvate decarboxylase (ARO10)
and that overexpresses each of the following enzymes:
   b. phospho-2-dehydro-3-deoxyheptonate aldolase (aroF)
   c. prephenate dehydrogenase (tyrA)
   d. 4-hydroxyphenylacetate 3-monooxygenase (hpaBC*) is grown in a medium comprising
      a metabolic precursor of phosphoenolpyruvate (PEP) and erythrose 4-phosphate (E4P), particularly glucose, and
      optionally, phenylalanine as a supplement;
   and hydroxytyrosol is extracted from said medium.
2. The method according to item 1, wherein the transgenic bacterial cell is of the genus Escherichia, particularly wherein the transgenic bacterial cell is of the species E. coli, more particularly wherein the transgenic bacterial cell is of the strain E. coli BL21.
3. The method according to any one of the preceding items, wherein the gene encoding the phenylpyruvate decarboxylase originates from yeast, particularly from S. cerevisiae.
4. The method according to any one of the preceding items, wherein the gene encoding the 4-hydroxyphenylacetate 3-monooxygenase originates from Escherichia, particularly from E. coli.
5. The method according to any one of the preceding items, wherein the gene encoding the 4-hydroxyphenylacetate 3-monooxygenase comprises amino acid substitutions S₂₁₀T, A₂₁₁₁L and Q₂₁₂E.
6. The method according to any one of the preceding items, wherein the transgenic bacterial cell does not overexpress any of the following proteins:
   alcohol dehydrogenase,
   DNA-binding transcriptional regulatory protein (tyrR),
   and tyrosine aminotransferase.
7. The method according to any one of the preceding items, wherein the only heterologously expressed gene of the transgenic bacterial cell is phenylpyruvate decarboxylase.
8. The method according to any one of the preceding items, wherein the overexpressed genes and the transgenes are introduced into the transgenic bacterial cell via one or several plasmid vector(s), particularly wherein
   phenylpyruvate decarboxylase, phospho-2-dehydro-3-deoxyheptonate aldolase and prephenate dehydrogenase are encoded by a medium-copy plasmid vector, and/or
   4-hydroxyphenylacetate 3-monooxygenase is encoded by a low-copy plasmid vector.
9. The method according to any one of the preceding items, wherein said transgenic bacterial cell comprises one or more plasmids encoding said heterologously expressed or overexpressed enzymes under control of a promoter sequence operable in said cell, particularly a T7 promoter (SEQ ID NO. 31), a lac promoter (SEQ ID NO. 32), a tac promoter (SEQ ID NO. 33) or a trc promoter (SEQ ID NO. 34), more particularly wherein
   the gene encoding 4-hydroxyphenylacetate 3-monooxygenase is under control of a T7 promoter, and/or
   the gene encoding phenylpyruvate decarboxylase is under control of a T7 promoter, and/or
   the gene encoding phospho-2-dehydro-3-deoxyheptonate aldolase is under control of a T7 promoter, and/or
   the gene encoding prephenate dehydrogenase is under control of a T7 promoter.
10. The method according to item 9, wherein the expression of said heterologous and/or overexpressed genes is induced by adding isopropyl-β-d-thiogalactopyranoside (IPTG), particularly at a concentration of ˜0.1 mM IPTG for 96 h.
11. The method according to any one of the preceding items, wherein said medium comprises 10 to 50 g/L of glucose, particularly 15 to 30 g/L of glucose.
12. The method according to any one of the preceding items, wherein the transgenes are codon-optimized for expression in said transgenic bacterial cell.
13. The method according to any one of the preceding items, wherein the medium comprises
   5-10 g/L Na₂HPO₄·2H₂O,
   2-4 g/L KH₂PO₄,
   0.25-1 g/L NaCl,
   0.5-1.5 g/L NH₄Cl,
   1-3% (w/v) glucose,
   0.01-0.05% (w/v) yeast extract,
   3-7 mM MgSO₄,
   0.005-0.02 g/L CaCl₂),
   0.5-2.0 g/L ascorbic acid, and
   antibiotics, particularly the antibiotics are 50-200 μg/mL ampicillin, 10-50 μg/mL kanamycin and 25-45 μg/mL chloramphenicol.
14. The method according to any one of the preceding items, wherein dodecanol is added to the medium, particularly ˜25% dodecanol (v/v) is added to the medium.
15. The method according to any one of the preceding items, wherein the cells are grown with ≥2% (v/v) of O₂, particularly with 2-4% (v/v) of 02.
16. The method according to any one of the preceding items, wherein
   a. the protein phenylpyruvate decarboxylase has at least 60%, 65%, 70%, 75%, 80%, 85%, 90%, 95% or >95% sequence identity with SEQ ID NO 1 and has a catalytic activity of at least 75% of the activity of SEQ ID NO 1 and/or
   b. the protein phospho-2-dehydro-3-deoxyheptonate aldolase has at least 60%, 65%, 70%, 75%, 80%, 85%, 90%, 95% or >95% sequence identity with SEQ ID NO 2 and has a catalytic activity of at least 75% of the activity of SEQ ID NO 2 and/or
   c. the protein prephenate dehydrogenase has at least 60%, 65%, 70%, 75%, 80%, 85%, 90%, 95% or >95% sequence identity with SEQ ID NO 3 and has a catalytic activity of at least 75% of the activity of SEQ ID NO 3 and/or
   d. the protein 4-hydroxyphenylacetate 3-monooxygenase has at least 60%, 65%, 70%, 75%, 80%, 85%, 90%, 95% or >95% sequence identity with SEQ ID NO 035 and has a catalytic activity of at least 75% of the activity of SEQ ID NO 035.
17. A transgenic cell as specified in any one of the preceding items. 18. A transgenic cell that heterologously expresses the following enzyme:
   a. phenylpyruvate decarboxylase (ARO10) and that overexpresses each of the following enzymes:
   b. phospho-2-dehydro-3-deoxyheptonate aldolase (aroF)
   c. prephenate dehydrogenase (tyrA)
and wherein each of the following genes is not expressed:
   i. pheAL (Bifunctional chorismate mutase/prephenate dehydratase);
   ii. feaB (Phenylacetaldehyde dehydrogenase).
19. A transgenic cell that heterologously expresses each of the following enzymes:
   a. phenylpyruvate decarboxylase (ARO10);
   b. uridine diphosphate dependent glycosyltransferase (UGT85A1);
and that overexpresses each of the following enzymes:
   c. phospho-2-dehydro-3-deoxyheptonate aldolase (aroF)
   d. prephenate dehydrogenase (tyrA)
and wherein each of the following genes is not expressed:
   i. pheAL (Bifunctional chorismate mutase/prephenate dehydratase)
   ii. feaB (Phenylacetaldehyde dehydrogenase).
20. A transgenic cell that heterologously expresses the following enzyme:
   a. phenylpyruvate decarboxylase (ARO10) and that overexpresses each of the following enzymes:
   b. phospho-2-dehydro-3-deoxyheptonate aldolase (aroF),
   c. prephenate dehydrogenase (tyrA);
   d. 4-hydroxyphenylacetate 3-monooxygenase (hpaBC*).
21. The transgenic cell according to any one of the preceding items 17 to 20, wherein the transgenic bacterial cell is of the genus Escherichia, particularly wherein the transgenic bacterial cell is of the species E. coli, more particularly wherein the transgenic bacterial cell is of the strain E. coli BL21.
22. The transgenic cell according to any one of the preceding items 17 to 21, wherein the gene encoding the phenylpyruvate decarboxylase originates from yeast, particularly from S. cerevisiae.
23. The transgenic cell according to any one of the preceding items 17 or 20 to 22, wherein the gene encoding the 4-hydroxyphenylacetate 3-monooxygenase originates from Escherichia, particularly from E. coli.
24. The transgenic cell according to any one of the preceding items 17 or 20 to 23, wherein the gene encoding the 4-hydroxyphenylacetate 3-monooxygenase comprises amino acid substitutions S₂₁₀T, A₂₁₁L and Q₂₁₂E.
25. The transgenic cell according to any one of the preceding items 17 or 19 or 21 to 22, wherein the gene encoding uridine diphosphate dependent glycosyltransferase originates from a plant, particularly from Arabidopsis, more particularly from A. thaliana.
26. The transgenic cell according to any one of the preceding items 17 to 25, wherein the transgenic bacterial cell does not overexpress any of the following proteins:
   alcohol dehydrogenase, (UniProtKB—P39451; EC:1.1.1.1),
   DNA-binding transcriptional regulatory protein (tyrR NCBI GenPept: NP_415839.1),
   and tyrosine aminotransferase, (UniProtKB—P04693, EC:2.6.1.57).
27. The transgenic cell according to any one of the preceding items 18 or 20 to 26, wherein the only heterologously expressed gene of the transgenic cell is phenylpyruvate decarboxylase.
28. The transgenic cell according to any one of the preceding items 19 or 21 to 26, wherein the only heterologously expressed genes of the transgenic cell are phenylpyruvate decarboxylase and uridine diphosphate dependent glycosyltransferase.
29. The transgenic cell according to any one of the preceding items 17 to 27, wherein the overexpressed genes and the transgenes are introduced into the transgenic bacterial cell via one or several plasmid vector(s), particularly wherein
   phenylpyruvate decarboxylase, phospho-2-dehydro-3-deoxyheptonate aldolase and prephenate dehydrogenase are encoded by a medium-copy plasmid vector, and/or
   uridine diphosphate dependent glycosyltransferase is encoded by a low-copy plasmid vector, and/or
   4-hydroxyphenylacetate 3-monooxygenase is encoded by a low-copy plasmid vector.
30. The transgenic cell according to any one of the preceding items 17 to 28, wherein transgenic bacterial cell comprises one or more plasmids encoding said heterologously expressed or overexpressed enzymes under control of a promoter sequence operable in said cell, particularly a T7 promoter (SEQ ID NO. 31), a lac promoter (SEQ ID NO. 32), a tac promoter (SEQ ID NO. 33) or a trc promoter (SEQ ID NO. 34), more particularly wherein
   the gene encoding uridine diphosphate dependent glycosyltransferase is under control of a trc promoter, and/or
   the gene encoding phenylpyruvate decarboxylase is under control of a T7 promoter, and/or
   the gene encoding phospho-2-dehydro-3-deoxyheptonate aldolase is under control of a T7 promoter, and/or
   the gene encoding prephenate dehydrogenase is under control of a T7 promoter; and/or
   the gene encoding 4-hydroxyphenylacetate 3-monooxygenase is under control of a T7 promoter.
31. The transgenic cell according to any one of the preceding items 17 to 29, wherein
   the protein phenylpyruvate decarboxylase has at least 60%, 65%, 70%, 75%, 80%, 85%, 90%, 95% or >95% sequence identity with SEQ ID NO 1 and has a catalytic activity of at least 75% of the activity of SEQ ID NO 1, and/or
   the protein phospho-2-dehydro-3-deoxyheptonate aldolase has at least 60%, 65%, 70%, 75%, 80%, 85%, 90%, 95% or >95% sequence identity with SEQ ID NO 2 and has a catalytic activity of at least 75% of the activity of SEQ ID NO 2, and/or
   the protein prephenate dehydrogenase has at least 60%, 65%, 70%, 75%, 80%, 85%, 90%, 95% or >95% sequence identity with SEQ ID NO 3 and has a catalytic activity of at least 75% of the activity of SEQ ID NO 3, and/or
   the protein uridine diphosphate dependent glycosyltransferase has at least 60%, 65%, 70%, 75%, 80%, 85%, 90%, 95% or >95% sequence identity with SEQ ID NO 4 and has a catalytic activity of at least 75% of the activity of SEQ ID NO 4, and/or
   the protein 4-hydroxyphenylacetate 3-monooxygenase has at least 60%, 65%, 70%, 75%, 80%, 85%, 90%, 95% or >95% sequence identity with SEQ ID NO 035 and has a catalytic activity of at least 75% of the activity of SEQ ID NO 035.

### DESCRIPTION OF FIGURES

FIG. 1 shows biosynthesis of tyrosol and salidroside in E. coli BL21 (DE3) using glucose as carbon source. To produce tyrosol, the genes aroF^{fbr}, tyrA^{fbr} and ScARO10* were cloned in a plasmid and transformed in E. coli BL21 (DE3) to yield tyrosol production strains. In order to produce salidroside, the gene AtUGT85A1 were cloned in different plasmids and transformed in E. coli BL21 (DE3) to yield salidroside production strains from tyrosol production strains. For salidroside production there was a dynamic control over the relevant biosynthetic genes, as indicated by triangle and circle symbols: filled triangles indicate the use of T7 promoter, while open triangles indicate the use of trc promoter; one circle indicate the use of low copy number plasmid, two circles indicate the use of medium copy number plasmid and three circles indicate the use of high copy number plasmid. Abbreviations: phosphoenolpyruvate (PEP); erythrose 4-phosphate (E4P); phospho-2-dehydro-3-deoxyheptonate aldolase (aroF^{fbr}); 3-deoxy-D-arabino-heptulosonate 7-phosphate (DAHP); prephenate dehydrogenase (tyrA^{fbr}); 4-hydroxyphenylpyruvate (4-HPP); phenylpyruvate decarboxylase from S. cerevisiae (ScARO10*); 4-hydroxyphenylacetaldehyde (4-HPAA); alcohol dehydrogenases (Ps); uridine diphosphate dependent glycosyltransferase from A. thaliana (AtUGT85A1).

FIG. 2 shows selection of the best phenylpyruvate decarboxylase (ScARO10*, EipdC and KpPDC) for tyrosol production from glucose in E. coli BL21 (DE3). a) Schematic overview of the pathway for tyrosol production from glucose. b) Tyrosol titer (g/L) for strains ST93, ST135 and ST136 induced with 0.1 mM of iPTG in M9Y medium. Cultures were sampled after 72 h of growth for tyrosol detection. Statistical analysis was performed by using Student's t test (*p<0.05, **p<0.01, ***p<0.001). All data represent the mean of n=3 biologically independent samples and error bars show standard deviation.

FIG. 3 shows the influence of overexpression of adhP* on tyrosol production from glucose in E. coli BL21 (DE3). a) Schematic overview of the pathway for tyrosol production from glucose. b) Tyrosol titer (g/L) for strains ST93, ST81 and ST114 induced with 0.1 mM of IPTG in M9Y medium. Cultures were sampled after 48 h of growth for tyrosol detection. The strain ST81 was grown at 22 and 30° C. to evaluate the effect on tyrosol titer. Statistical analysis was performed by using Student's t test (*p<0.05, **p<0.01, ***p<0.001). All data represent the mean of n=3 biologically independent samples and error bars show standard deviation.

FIG. 4 shows engineering aromatic amino acid pathways to improve tyrosol production from glucose in E. coli BL21 (DE3). a) Schematic overview of the pathway for tyrosol production from glucose. b) Tyrosol titer (g/L) for strains ST170 and 191 harbouring knockouts on feaB and pheAL genes induced with 0.1 mM of IPTG in M9Y medium with or without phenylalanine supplementation. Cultures were sampled after 96 h of growth for tyrosol detection. Statistical analysis was performed by using Student's t test (*p<0.05, **p<0.01, ***p<0.001). All data represent the mean of n=3 biologically independent samples and error bars show standard deviation. ARO10*_aroF^{fbr}_tyrA^{fbr} corresponds to plasmid pET-21a(+)_ScARO10*_aroF^{fbr}_tyrA^{fbr} and adhP* corresponds to pET-28a(+)_adhP*.

FIG. 5 shows the effect of different expression level of AtUGT85A1 on salidroside production from glucose in E. coli BL21 (DE3). a) Schematic overview of the pathway for salidroside production from glucose. b) Salidroside and tyrosol titers (g/L) for strains ST92, 116, 131 and 176 induced with 0.1 mM of IPTG in M9Y medium. Cultures were sampled after 48 h of growth for salidroside and tyrosol detection. Statistical analysis was performed by using Student's t test (*p<0.05, **p<0.01, ***p<0.001). All data represent the mean of n=3 biologically independent samples and error bars show standard deviation

FIG. 6 shows engineering of aromatic amino acid pathways to improve salidroside production from glucose in E. coli BL21 (DE3). a) Schematic overview of the pathway for salidroside production from glucose. b) Salidroside titer (g/L) for strains ST172 and ST178 induced with 0.1 mM of IPTG in M9Y medium with or without phenylalanine supplementation. Cultures were sampled after 96 h of growth for salidroside detection. Statistical analysis was performed by using Student's t test (*p<0.05, **p<0.001). All data represent the mean of n=3 biologically independent samples and error bars show standard deviation.

FIG. 7. The effect of different expression level of hpaBC* on hydroxytyrosol production from glucose in E. coli BL21 (DE3). a) Schematic overview of the pathway for hydroxytyrosol production from glucose. b) Hydroxytyrosol titer (g/L) for strains ST76, 119 and 132 induced with 0.1 mM of IPTG in M9Y medium supplemented with 1 g/L of ascorbic acid. Cultures were sampled after 48 h of growth for hydroxytyrosol detection. Statistical analysis was performed by using Student's t test (*p<0.05, **p<0.01, ***p<0.001). All data represent the mean of FIG. 8. The effect of different expression level of hpaBC* on hydroxytyrosol production from glucose in E. coli BL21 (DE3). a) Schematic overview of the pathway for hydroxytyrosol production from glucose. b) Hydroxytyrosol titer (g/L) for strains ST119 and 132 induced with 0.1 mM of IPTG in M9Y medium supplemented with 1 g/L of ascorbic acid and addition or not of 25% (v/v) of 1-dodecanol. Cultures were sampled after 48 h of growth for hydroxytyrosol detection. Statistical analysis was performed by using Student's t test. All data represent the mean of n=3 biologically independent samples and error bars show standard deviation (see materials and methods).

### MATERIAL AND METHODS

Cloning Strategy

E. coli DH5a cells (New England BioLabs, Massachusetts, USA) were used for gene cloning and vector propagation. This strain was cultured in Luria-Bertani (LB) medium (10 g/L tryptone, 5 g/L yeast extract, 10 g/L NaCl) with the appropriate antibiotic concentration. The solid version of this medium included 20 g/L of agar. All cultivations were performed at 37° C. and, in the case of liquid cultures, under shaking conditions (200 rpm). For long-term storage, glycerol was added to a final concentration of 30% (v/v) to overnight cultures in selective media and kept in a −80° C. freezer.

The genes used in this study were amplified by polymerase chain reaction (PCR) using Phusion High-Fidelity DNA Polymerase (Thermo Scientific, Waltham, USA) in a LifeECO Thermal Cycler. All primers were purchased from Integrated DNA Technologies (Coralville, USA). DNA fragments were purified using DNA Clean and Concentrator DNA Kit (Zymo Research, Irvine, USA).

Plasmids were extracted using Plasmid Miniprep Kit (Zymo Research). All digestions were performed using the appropriate FastDigest® restriction endonucleases (Thermo Scientific). Ligations were performed with T4 DNA Ligase (Thermo Scientific) and transformed in chemically competent E. coli DH5a cells and E. coli BL21 (DE3) using Mix & Go E. coli Transformation Kit & Buffer Set (Zymo Research). The success of ligation was checked through colony PCR using DreamTaq (Thermo Scientific) and further confirmed by sequencing (StabVida, Lisbon, Portugal). Protocols were performed in accordance with manufacturer's instructions.

The tyrA^{fbr} gene and the codon-optimized genes ScARO10*, KpPDC, EipdC and AtUGT85A1 were purchased from IDT DNA Technology (Coralville, USA) and cloned in pET-21a(+) vector (Novagen, Darmstadt, Germany) in the case of tyrA^{fbr} and ScARO10*, in pJET1.2 vector (CloneJET PCR Cloning Kit, Thermo Scientific) in the case of KpPDC and EipdC, and in pET-28a(+) vector (Novagen, Darmstadt, Germany) for the case of UGT gene. aroF^{fbr} and hpaBC* genes were amplified from E. coli BL21 (DE3) genomic DNA from New England BioLabs (Massachusetts, USA). hpaBC* gene was mutated in S₂₁₀T, A₂₁₁₁L and Q₂₁₂E of HpaB subunit, in order to improve the activity for tyrosol (Chen, 2019). adhP* was kindly provided by Prof. Isabel Rocha group (University of Minho, Portugal).

Plasmid Construction and Bacterial Strains

The plasmids pET-21a(+), pET-28a(+), pACYCDuet and pRSFDuet (Novagen, Darmstadt, Germany) were used to provide individual expression of each protein under the control of the T7lac promoter and a ribosome binding site (RBS). All the plasmids were constructed by traditional molecular biology techniques and the success of the plasmid constructions was confirmed by colony PCR and sequencing the regions of interest with the appropriate primers.

E. coli DH5a was used as a host for gene cloning and plasmid propagation while E. coli BL21 (DE3), the parent strain, was engineered to produce tyrosol, salidroside, and hydroxytyrosol. For all the strains, positive transformants were isolated in LB agar plates, containing the appropriate antibiotic concentrations (100 μg/mL ampicillin, 30 μg/mL kanamycin and 34 μg/mL chloramphenicol) and incubated at 37° C., overnight. To confirm the success of the transformation, a few transformant colonies were cultivated in LB medium with appropriate antibiotics, overnight. Afterwards, plasmids were extracted, digested with appropriate restriction enzymes and the correct fragment lengths were confirmed by running the digestion in a 1% (w/v) agarose gel.

Construction of Tyrosol Plasmids and Strains

The plasmid pET-21a(+) (Novagen), with ampicillin resistance marker, was used to clone the genes adhP*, aroF^{fbr}, tyrA^{fbr} and the codon-optimized gene, ScARO10*. The optimized phenylpyruvate decarboxylase gene ScARO10* was amplified by PCR using the primer pair ARO10*_pet_fw/ARO10*_RBS_rev (primers are shown in Table 1) and the plasmid pET-21a(+) was amplified by PCR using the primer pair pet21a_fw/pet21a_rev. These two fragments were fused using circular polymerase extension cloning (CPEC) (Quan, J. et al, Nat Protoc 6, 242-251 (2011)). Then, this PCR product was amplified by PCR using the primers ARO10*_pet_fw and ARO10_hindiii_rev, restricted with Ndel and Hindlll and cloned into the plasmid pET-21a(+), also restricted with these enzymes, originating pET-21a(+)_ScARO10*. The PCR product for aroF^{fbr}, with the mutation D147N, was amplified by PCR in two fragments, using the primer pairs aroF_fbr_RBS_fwlaroF_D147N_rev and aroF_D147N_fwlaroF_fbr_RBS_rev. These two fragments were fused using PCR technique with the primer pair aroF_fbr_RBS_fwlaroF_fbr_RBS_rev, and was restricted and ligated into Hindlll and Notl restriction sites of the previous construction, originating pET-21a(+)_ScARO10*_aroF^{fbr}. The chorismate mutase or prephenate dehydrogenase gene, tyrA^{fbr}, with the mutations M531 and A354V was ordered from IDT DNA Technology (USA) and restricted with Notl and Xhol in order to be cloned into the previous construction, originating pET-21a(+)_ScARO10*_aroF^{fbr}_tyrA^{fbr}. The alcohol dehydrogenase gene, adhP*, was amplified by PCR from the plasmid pET-28a(+)_adhP*, that was kindly provided by Prof. Isabel Rocha group (University of Minho, Portugal) with the primers Tyr2_adhp_JO_fw and Tyr2_adhp_JO_rev, after the plasmid pET-21a(+)_ScARO10*_aroF^{fbr}_tyrA^{fbr} was restricted with Notl and then the amplified fragment and the plasmid were ligated using the In-Fusion® HD Cloning Plus Kit (TaKaRa, France), forming pET-21a (+)_ScARO10* aroF^{fbr}_adhP*_tyrA^{fbr}.

TABLE 1
Sequences of primers used in the cloning
procedures of tyrosol production strains in
this study (⁺restriction sites are underlined).
Abbreviations: fw-forward and rev-reverse.

| | | Restriction |
|---|---|---|
| Primer | Sequence (SEQ ID NO.) | Sites⁺ |
| pet21a_fw | ctcgagcaccaccaccac (SEQ ID NO. 13) | — |
| ARO10*_pet_fw | actttaagaaggagatatacatatgGCTCCGGTTACCATCG | — |
| | (SEQ ID NO. 14) | |
| pet21a_rev | Ctcgagcaccaccaccac (SEQ ID NO. 15) | — |
| ARO10*_RBS_ | AACAAAATTATTTCTATTAggtaccTTATTTTTTGTTACGTTTCA | KpnI |
| rev | GAGCAG (SEQ ID NO. 16) | |
| ARO10_hindiii_ | cccAAGCTTTTATTTTTTGTTACGTTTCAGAGCAG (SEQ ID | HindIII |
| rev | NO. 17) | |
| aroF_fbr_RBS_ | GTTTAACTTTAtaaggaggaaaaaaaATGcaaaaagacgcgctga | — |
| fw | (SEQ ID NO. 18) | |
| aroF_D147N_fw | cggaagcgttaaatccgaatag (SEQ ID NO. 19) | — |
| aroF_D147N_ | ctattcggatttaacgcttccg (SEQ ID NO. 20) | — |
| rev | | |
| aroF_fbr_RBS_ | AACAAAATTATTTCTATTAggtaccttaagccacgcgagccgtc | KpnI |
| rev | (SEQ ID NO. 21) | |
| Tyr2_adhp_JO_ | GTGGCTTAAGCGGCCTAATACGACTCACTATAGGGGAATT | — |
| fw | (SEQ ID NO. 22) | |
| tyr2_adph_JO_ | TTTCTATTAGCGGCCGAATTCTTAGTGACGGAAATCAATC | — |
| rev | (SEQ ID NO. 23) | |
| pet28a_RBS_ | AACAAAATTATTTCTATTAggtaccggggaattgttatccgctc | |
| rev | (SEQ ID NO. 24) | KpnI |
| RBS_linker_st7 | ggtaccTAATAGAAATAATTTTGTTTAACTTTAtaaggaggaaaaaaa | KpnI |
| fw | (SEQ ID NO. 25) | |
| tyrA_fbr_pet_ | cagtggtggtggtggtggtgctcgagTTACTGGCGATTGTCATTCG | XhoI |
| rev | (SEQ ID NO. 26) | |

Alternatively, the plasmid pET-28a(+) (Novagen), containing kanamycin resistance gene, was also used to clone the genes aroF^{fbr} and tyrA^{fbr}. For that, the pET-28a(+) plasmid was amplified by PCR using the primers pet21a_fw and pet28a_RBS_rev and the aroF^{fbr} gene was amplified from pET-21a(+)_ScARO10*_aroF^{fbr}_tyrA^{fbr} plasmid, using the primers RBS_linker_st7_fw and aroF_fbr_RBS_rev. After, both fragments were merged using CPEC, originating pET-28a(+)_aroF^{fbr}. Afterwards, this plasmid was amplified by PCR with the primers pet21a_fw and aroF_fbr_RBS_rev and the tyrA^{fbr} gene was amplified from pET-21a(+)_ScARO10*_aroF^{fbr}_tyrA^{fbr} plasmid, using the primers RBS_linker_st7 fw and tyrA_fbr pet rev. Finally, these two fragments were fused using the CPEC strategy, forming pET-28a(+)_aroF^{fbr} tyrA^{fbr}.

Furthermore, two alternative decarboxylases encoded by EipdC and KpPDC genes from Enterobacter sp. and Komagataella phaffii, respectively, were tested instead of ScARO10*. For that, the synthetic genes previously cloned into pJET1.2 (Thermo Scientific) were restricted with Xbal and Hindlll and cloned into the plasmid pET-21a(+)_ScARO10*_aroF^{fbr}_tyrA^{fbr}, also restricted with these enzymes, originating pET-21a (+)_EipdC_aroF^{fbr} tyrA^{fbr} and pET-21a(+)_KpPDC_aroF^{fbr}_tyrA^{fbr}, respectively.

The plasmids and tyrosol production strains constructed and used in this work are listed in Table 2.

TABLE 2
Plasmids and strains used or engineered for tyrosol production in this work.

| | | Source or |
|---|---|---|
| Plasmids and strains | Relevant genotype and characteristics | reference |
| pET-21a(+) | Amp^{R} | Novagen |
| pET-21a(+)_ScARO10* | Amp^{R}, ScARO10* | This study |
| pET-21a(+)_ScARO10*_aroF^{fbr}_tyrA^{fbr} | Amp^{R}, ScARO10*, aroF^{fbr}, tyrA^{fbr} | This study |
| PET- | Amp^{R}, ScARO10*, aroF^{fbr}, adhP*, | This study |
| 21a(+)_ScARO10*_aroF^{fbr}_adhP*_tyrA^{fbr} | tyrA^{fbr} | |
| pET-21a(+)_KpPDC_aroF^{fbr}_tyrA^{fbr} | Amp^{R}, KpPDC, aroF^{fbr}, tyrA^{fbr} | This study |
| pET-21a(+)_EipdC_aroF^{fbr}_tyrA^{fbr} | Amp^{R}, EipdC, aroF^{fbr}, tyrA^{fbr} | This study |
| pET-28a(+) | Kan^{R} | Novagen |
| pET-28a(+)_aroF^{fbr}_tyrA^{fbr} | Kan^{R}, aroF^{fbr}, tyrA^{fbr} | This study |
| pET-28a(+)_adhP* | Kan^{R}, adhP* | (a) |
| E. coli DH5α | fhuA2 Δ(argF-lacZ)U169 phoA | NEB |
| | glnV44 Φ80 Δ(lacZ)M15 gyrA96 | |
| | recA1 relA1 endA1 thi-1 hsdR17 | |
| E. coli BL21 (DE3) | fhuA2 [lon] ompT gal (λ DE3) [dcm] | NEB |
| | ΔhsdS λ DE3 = λ sBamHlo ΔEcoRI- | |
| | B int::(lacI::PlacUV5::T7 gene1) i21 | |
| | Δnin5 | |
| E. coli BL21 (DE3) ΔpheALΔfeaB | E. coli BL21 (DE3) with knockouts in | SilicoLife |
| | the pheAL and feaB genes | |
| ST53 | E. coli BL21 (DE3) with | This study |
| | pET-21a(+)_ScARO10* | |
| ST93 | E. coli BL21 (DE3) with | This study |
| | pET- | |
| | 21a(+)_ScARO10*_aroF^{fbr}_tyrA^{fbr} | |
| ST96 | ST53 with pET- | This study |
| | 28a(+)_aroF^{fbr}_tyrA^{fbr} | |
| ST81 | ST96 with pET-28a(+)_adhP* | This study |
| ST114 | E. coli BL21 (DE3) with | This study |
| | pET-21a(+)_KpPDC_aroF^{fbr}_tyrA^{fbr} | |
| ST135 | E. coli BL21 (DE3) with | This study |
| | pET-21a(+)_EipdC_aroF^{fbr}_tyrA^{fbr} | |
| ST170 | E. coli BL21 (DE3) ΔpheALΔfeaB with | This study |
| | pET- | |
| | 21a(+)_ScARO10*_aroF^{fbr}_tyrA^{fbr} | |
| | and pET-28a(+)_adhP* | |
| ST191 | E. coli BL21 (DE3) ΔpheALΔfeaB with | This study |
| | pET- | |
| | 21a(+)_ScARO10*_aroF^{fbr} tyrA^{fbr} | |

(a) Plasmid kindly provided by Prof. Isabel Rocha group (University of Minho, Portugal).

Construction of Salidroside Plasmids and Strains

The plasmid pET-28a(+) was used to clone the codon optimized gene AtUGT85A1, corresponding to the final step of the proposed pathway, which consists in the conversion of tyrosol into salidroside. The AtUGT85A1 gene was amplified by PCR using the primers UGT85a1_ncoi_fw and UGT85A1_ (primers are shown in Table 3) with restriction sites to Ncol and BamHI and cloned in pET-28a(+), originating pET-28a(+)_AtUGT85A1.

Additionally, to test different plasmid copy number, the AtUGT85A1 gene was cloned in the plasmids pACYCDuet and pRSFDuet, with chloramphenicol and kanamycin resistance marker, respectively. To construct pACYCDuet_AtUGT85A1 and pRSFDuet_AtUGT85A1 plasmids the AtUGT85A1 gene was extract with Ndel and Xhol from pET28a(+)_AtUGT85A1 plasmid, and cloned in pACYCDuet and pRSFDuet, respectively, also digested with these enzymes.

Moreover, to increase salidroside production the T7lac promoter in pACYCDuet_AtUGT85A1 was replaced by trc promoter, using PCR technique with primers pacyc_trc_mc2_fw and pacyc_trc_mc2_rev, originating pACYCDuet_trc-promoter_AtUGT85A1.

TABLE 3
Sequences of primers used in the cloning
procedures of salidroside production
strains in this work (⁺restriction sites
are underlined). Abbreviations: fw-forward
and rev-reverse.

| | | Restriction |
|---|---|---|
| Primer | Sequence | Sites⁺ |
| UGT85a1_ | cccccatgGGATCACAGATCAT | NcoI |
| ncoi_fw | ACAC | |
| | (SEQ ID NO. 27) | |
| UGT85A1_ | ccggatccTTAGTCCTGGCTTT | BamHI |
| bamhi_rev | TC (SEQ ID NO. 28) | |
| pacyc_trc_ | TTGACAATTAATCATCCGGCT | |
| mc2_fw | CGTATAATGggaattgtgag | |
| | cggataacaattc | |
| | (SEQ ID NO. 29) | |
| pacyc_trc_ | CATTATACGAGCCGGATGATTA | |
| mc2_rev | ATTGTCAAgcaggagtcg | |
| | cataagggagagc | |
| | (SEQ ID NO. 30) | |

The plasmids and salidroside production strains constructed and used in this study are listed in Table 4.

TABLE 4
Plasmids and strains used or engineered for salidroside production in this study.

| | | Source or |
|---|---|---|
| Plasmids and strains | Relevant genotype and characteristics | reference |
| pET-21a(+)_ScARO10* | Amp^{R}, ScARO10* | This study |
| pET-21a(+)_ScARO10*_aroF^{fbr}_tyrA^{fbr} | Amp^{R}, ScARO10*, aroF^{fbr}, tyrA^{fbr} | This study |
| pET- | Amp^{R}, ScARO10*, aroF^{fbr}, adhP*, tyrA^{fbr} | This study |
| 21a(+)_ScARO10*_aroF^{fbr}_adhP*_tyrA^{fbr} | | |
| pET-28a(+)_AtUGT85A1 | Kan^{R}, AtUGT85A1 | This study |
| pACYCDuet_AtUGT85A1 | Chlo^{R}, AtUGT85A1, T7lac promoter | This study |
| pACYCDuet_trc-pm_AtUGT85A1 | Chlo^{R}, AtUGT85A1, trc promoter | This study |
| pRSFDuet_AtUGT85A1 | Kan^{R}, AtUGT85A1 | This study |
| E. coli DH5α | fhuA2 Δ(argF-lacZ)U169 phoA glnV44 | NEB |
| | Φ80 Δ(lacZ)M15 gyrA96 recA1 relA1 | |
| | endA1 thi-1 hsdR17 | |
| E. coli BL21 (DE3) | fhuA2 [lon] ompT gal (λ DE3) [dcm] | NEB |
| | ΔhsdS λ DE3 = λ sBamHlo ΔEcoRI-B | |
| | int::(lacI::PlacUV5::T7 gene1) i21 Δnin5 | |
| E. coli BL21 (DE3) ΔpheALΔfeaB | E. coli BL21 (DE3) with knockouts in the | SilicoLife |
| | pheAL and feaB genes | |
| ST95 | E. coli BL21 (DE3) with | This study |
| | pET-21a(+)_ScARO10* and | |
| | pET-28a(+)_AtUGT85A1 | |
| ST92 | E. coli BL21 (DE3) with | This study |
| | pET-21a(+)_ScARO10*_aroF^{fbr}_tyrA^{fbr} | |
| | and pET-28a(+)_AtUGT85A1 | |
| ST116 | E. coli BL21 (DE3) with | This study |
| | pET-21a(+)_ScARO10*_aroF^{fbr} tyrA^{fbr} | |
| | and pACYCDuet_AtUGT85A1 | |
| ST117 | E. coli BL21 (DE3) with pET- | This study |
| | 21a(+)_ScARO10*_aroF^{fbr}_adhP*_tyrA^{fbr} | |
| | and pACYCDuet_AtUGT85A1 | |
| ST131 | E. coli BL21 (DE3) with | This study |
| | pET-21a(+)_ScARO10*_aroF^{fbr}_tyrA^{fbr} | |
| | and pRSFDuet_AtUGT85A1 | |
| ST176 | E. coli BL21 (DE3) with | This study |
| | pET-21a(+)_ScARO10*_aroF^{fbr}_tyrA^{fbr} | |
| | and pACYCDuet_trc-pm_AtUGT85A1 | |
| ST172 | E. coli BL21 (DE3) ΔpheALΔfeaB with | This study |
| | pET-21a(+)_ScARO10*aroF^{fbr}_tyrA^{fbr} | |
| | and pACYCDuet_AtUGT85A1 | |
| ST178 | E. coli BL21 (DE3) ΔpheALΔfeaB with | This study |
| | pET-21a(+)_ScARO10*_aroF^{fbr}_tyrA^{fbr} | |
| | and pACYCDuet_trc-pm_AtUGT85A1 | |

Construction of Hydroxytyrosol Plasmids and Strains

The plasmid pET-28a(+) was used to clone the hpaBC* gene with mutations in S₂₁₀T, A₂₁₁₁L and Q₂₁₂E of HpaB subunit, which enzyme is responsible for conversion of tyrosol into hydroxytyrosol. These mutations, identified by Chen and his co-workers, improve the activity and specificity of HpaB towards tyrosol. The hpaBC* gene was amplified by PCR in two fragments to insert the given mutations using the primer pairs hpaB_rbs_xbailhpab_210_2_rev and hpab_210_2_fwlhpac_bamhi_rev, using genomic DNA of E. coli BL21 (DE3) as template (primers are shown in Table 5). These two fragments were fused using PCR technique with the primer pair hpaB_rbs_xbailhpac_bamhi_rev, restricted and ligated into Xbal and BamHI restriction sites of the plasmid pET-28a(+), forming pET-28a(+)_hpaBC*.

In addition, to test the influence of different plasmid copy number, the hpaBC*gene was cloned in the plasmids pACYCDuet and pRSFDuet with chloramphenicol and kanamycin resistance marker, respectively. For both cases, the hpaBC* gene was extract from pET-28a(+)_hpaBC* plasmid, restricted and ligated into Ndel and Xhol restriction sites of each plasmid, originating pACYCDuet_hpaBC* and pRSFDuet_hpaBC*.

TABLE 5
Sequences of primers used in the cloning
procedures of hydroxytyrosol production
strains in this study.
Abbreviations: fw-forward and rev-reverse.

| | | Restriction |
|---|---|---|
| Primer | Sequence | Sites |
| hpaB_rbs_ | cctctagattaactttaagaagg | XbaI |
| xbai | agtatacatATGAAACCAGAAGA | |
| | TTTCCGc (SEQ ID NO. 38) | |
| hpab_210_ | CGGCACCCTGGAAGTGATGGGCG | - |
| 2_fw | AAAACCCGGAC (SEQ | |
| | ID NO. 39) | |
| hpab_210_ | CATCACTTCCAGGGTGCCGAAGC | — |
| 2_rev | CAATCATGTTGTAG | |
| | (SEQ ID NO. 40) | |
| hpac_ | CCggatccTTAAATCGCAGCTTC | BamHI |
| bamhi_ | CATTTCCAG | |
| rev | (SEQ ID NO. 41) | |

The plasmids and hydroxytyrosol production strains constructed and used in this work are listed in Table 6.

TABLE 6
Plasmids and strains used or engineered for hydroxytyrosol production in this study.

| | | Source or |
|---|---|---|
| Plasmids and strains | Relevant genotype and characteristics | reference |
| pET- | Amp^{R}, ScARO10*, aroF^{fbr}, tyrA^{fbr} | This study |
| 21a(+)_ScARO10*_aroF^{fbr}_tyrA^{fbr} | | |
| pET-28a(+)_hpaBC* | Kan^{R}, hpaBC* | This study |
| pACYCDuet_hpaBC* | Chlo^{R}, hpaBC* | This study |
| pRSFDuet_hpaBC* | Kan^{R}, hpaBC* | This study |
| E. coli DH5 α | fhuA2 Δ (argF-lacZ)U169 phoA glnV44 Φ80 Δ | NEB |
| | (lacZ)M15 gyrA96 recA1 relA1 endA1 thi-1 | |
| | hsdR17 | |
| E. coli BL21 (DE3) | fhuA2 [lon] omp T gal (λ DE3) [dcm] ΔhsdS λ | NEB |
| | DE3 = λ sBamHlo ΔEcoRI-B | |
| | int::(lacI::PlacUV5::T7 gene1) i21 Δnin5 | |
| ST76 | E. coli BL21 (DE3) with | This study |
| | pET-21a(+)_ScARO10*_aroF^{fbr}_tyrA^{fbr} and | |
| | pET-28a(+)_hpaBC* | |
| ST119 | E. coli BL21 (DE3) with | This study |
| | pET-21a(+)_ScARO10*_aroF^{fbr}_tyrA^{fbr} and | |
| | pACYCDuet_hpaBC* | |
| ST132 | E. coli BL21 (DE3) with | This study |
| | pET-21a(+)_ScARO10*_aroF^{fbr}_tyrA^{fbr} and | |
| | pRSFDuet_hpaBC* | |

Strain Maintenance and Cultivation Media

All strains were cultivated in LB broth medium (10 g/L tryptone, 5 g/L yeast extract, 10 g/L NaCl) and M9Y medium, which contained 1×M9 minimal salts (Na₂HPO₄·2H₂O, 8.5 g/L; KH₂PO₄, 3.0 g/L; NaCl, 0.5 g/L; NH₄Cl, 1.0 g/L) and 2% (w/v) glucose, and was supplemented with 0.025% (w/v) yeast extract, 5 mM MgSO₄, 0.011 g/L CaCl₂) and with the appropriate antibiotic concentrations (100 μg/mL ampicillin, 30 μg/mL kanamycin and 34 μg/mL chloramphenicol). Additionally, strains with background of E. coli BL21 (DE3) LpheALLfeaB were supplemented with 20 mg/L of phenylalanine.

A single colony of the engineered E. coli strain was used to inoculate 10 ml liquid LB medium containing appropriate antibiotics and allowed to grow overnight at 37° C. with agitation of 200 rpm. Then, the precultures were transferred to 250 mL shake flask with 50 mL of LB medium containing the appropriate antibiotic, with an initial optical density (00600) of 0.1. Firstly, the cultures were cultivated on a rotary shaker at 200 rpm and 37° C. until cell density (00600) reached 0.6-0.8. At this point, in the case of tyrosol and salidroside, cells were collected by centrifugation (6000 rpm for 10 min), resuspended in 50 ml M9Y medium with suitable antibiotics and the gene expression was induced with isopropyl 1-thio-β-D-galactopyranoside (IPTG) at a final concentration of 0.1 or 1 mM. After induction, the cultures were incubated at 22 or 30° C. and with agitation of 200 rpm. Samples of broth were collected at time 0, induction time 24, 48, 72, 96 and 121 h for HPLC analysis and cell density measurement. For hydroxytyrosol, cells were cultivated as stated above with some changes: a) addition of 1 g/L of ascorbic acid; b) addition or absence of 12.5 ml of 1-dodecanol to the growth medium at 16 h of induction. These formulations aimed to improve hydroxytyrosol recovery. Samples of broth were collected at time 0, induction time 24 and 48 for high-performance liquid chromatography (HPLC) analysis and cell density measurement. All the experiments were performed in triplicate and the samples were analysed by HPLC and nuclear magnetic resonance spectroscopy (NMR).

Analytical Methods

The tyrosol, salidroside, hydroxytyrosol, glucose and organic acids content of the fermentation medium were analysed using HPLC. NMR technique was used to confirm the presence of tyrosol, salidroside and hydroxytyrosol in the medium samples and for quantification of hydroxytyrosol in the 1-dodecanol fraction of the biphasic growth.

For each sampling, 1 mL of broth were removed from the culture and centrifuged at 15000 rpm for 10 min to separate cells from the medium. Next, the supernatant was filtered through a membrane filter with a pore size of 0.22 μm into HPLC vials and stored at −20° C. until further analysis. Tyrosol, salidroside and hydroxytyrosol concentrations were quantified by an HPLC apparatus from SHIMADZU (Kyoto, Japan) model Nexera X2 equipped with DAD SPD-M20A detector, also from SHIMADZU. The samples were analysed using a Kinetex® C18 column (150 mm×2.1 mm; particle size, 1.7 μm) from Phenomenex (California, USA). For the analysis of tyrosol and salidroside, a 5 μl sample of the fermentation supernatant was applied to the column, along with the mobile phases included solvent A (0.1% formic acid in H₂O) and solvent B (acetonitrile with 0.1% formic acid). Each sample was eluted at 30° C., with a flow rate of 0.5 ml/min and under the following HPLC conditions: solvent B concentration was maintained at 5% for 1 min, then increased from 5% to 9% over 4 min, after increased from 9% to 30% during 5 min, remained at 30% for 6 min and finally decreased from 30% to 5% over 2 min. The compounds were detected at 280 nm. In these conditions, the retentions time of tyrosol and salidroside were 7 and 5 min, respectively. To quantify tyrosol and salidroside in the culture medium, calibration curves were generated with a series of known concentrations of the tyrosol standard (Fisher, USA) and salidroside standard (Sigma-Aldrich, USA) dissolved in water. The R² coefficients for the calibration curves were >0.99. For the analysis of hydroxytyrosol, a 10 μl sample of the fermentation supernatant was applied to the column, along with the mobile phases included solvent A (0.5% acetic acid in H₂O) and solvent B (100% acetonitrile). Each sample was eluted at 30° C., with a flow rate of 0.3 ml/min and under the following HPLC conditions: solvent B concentration was maintained at 5% for 2 min, then increased from 5% to 9% over 2 min, after increased from 9% to 30% over 6 min, then was maintained at 30% for 4 min and finally decreased from 30% to 5% over 2 min. The hydroxytyrosol was detected at 280 nm with the retention time of 8 min. To quantify hydroxytyrosol in the culture medium, calibration curves were generated with a series of known concentrations of the hydroxytyrosol standard (TCI, Japan) dissolved in water. The R² coefficients for the calibration curves were >0.99.

Quantitative analysis of glucose and fermentation products were performed using HPLC apparatus from Jasco (Japan) model LC-Netll/ADC equipped with UV-2075 Plus and RI-4030 Plus detectors, also from Jasco. The samples were analysed using an Aminex HPX-87H column (300 mm×7.7 mm) from Bio-Rad (USA), which was kept at 60° C. and 0.5 mM H2504 was used as mobile phase with a flow rate of 0.5 mL/min. Glucose and ethanol were detected with a refractive index (RI) detector (4030, Jasco) and organic acids (acetate, formate, lactate, succinate and pyruvate) were detected at 210 nm using the UV detector. Calibration curves were obtained by injecting standards with known concentrations for each metabolite. Metabolite concentrations in samples were calculated by comparing the peak areas of the samples with the calibration curves. The R² coefficients for the calibration curves were >0.99.

Hydroxytyrosol in the 1-dodecanol fraction of biphasic growth was quantified by a proton magnetic resonance spectroscopy (¹H) using a NMR device apparatus from BRUKER (USA) model Avance II 400 MHz spectrometer. To do so, 300 μl of 1-dodecanol fraction was diluted in 300 μl of deuterated chloroform plus 5 μl of a 250 mM formate solution (internal standard). To confirm the production of tyrosol, hydroxytyrosol and salidroside, positive samples analysed in the HPLC were promptly transferred to an NMR tube with 10% (v/v) of D₂O and read in the spectrometer referred above.

All cell optical density measurements (00600) were performed using the NanoDrop One spectrophotometer from Thermo Fisher (USA).

Statistical Analysis

All experiments were independently conducted three times. Experimental data are represented by the mean±standard deviation. Student's t test was used to conduct statistical analyses. Differences between engineered strains were considered significant when the P value was <0.05.

Sequences

Protein sequences:

TABLE 7
Listing of protein sequences.

| EC number/ | Protein/ | Sequence |
|---|---|---|
| Uniprot ID | Organism | (SEQ ID NO.) |
| EC: | Phenylpyruvate | MAPVTIEKFVNQEERHLVSN |
| 4.1.1.80/ | decarboxylase | RSATIPFGEYIFKRLLSIDT |
| Q06408 | Saccharomyces | KSVFGVPGDFNLSLLEYLYS |
| | cerevisiae | PSVESAGLRWVGTCNELNAA |
| | | YAADGYSRYSNKIGCLITTY |
| | | GVGELSALNGIAGSFAENVK |
| | | VLHIVGVAKSIDSRSSNFSD |
| | | RNLHHLVPQLHDSNFKGPNH |
| | | KVYHDMVKDRVACSVAYLED |
| | | IETACDQVDNVIRDIYKYSK |
| | | PGYIFVPADFADMSVTCDNL |
| | | VNVPRISQQDCIVYPSENQL |
| | | SDIINKITSWIYSSKTPAIL |
| | | GDVLTDRYGVSNFLNKLICK |
| | | TGIWNFSTVMGKSVIDESNP |
| | | TYMGQYNGKEGLKQVYEHFE |
| | | LCDLVLHFGVDINEINNGHY |
| | | TFTYKPNAKIIQFHPNYIRL |
| | | VDTRQGNEQMFKGINFAPIL |
| | | KELYKRIDVSKLSLQYDSNV |
| | | TQYTNETMRLEDPTNGQSSI |
| | | ITQVHLQKTMPKFLNPGDVV |
| | | VCETGSFQFSVRDFAFPSQL |
| | | KYISQGFFLSIGMALPAALG |
| | | VGIAMQDHSNAHINGGNVKE |
| | | DYKPRLILFEGDGAAQMTIQ |
| | | ELSTILKCNIPLEVIIWNNN |
| | | GYTIERAIMGPTRSYNDVMS |
| | | WKWTKLFEAFGDFDGKYTNS |
| | | TLIQCPSKLALKLEELKNSN |
| | | KRSGIELLEVKLGELDFPEQ |
| | | LKCMVEAAALKRNKK |
| | | (SEQ ID NO. 1) |
| EC: | phospho-2- | MQKDALNNVHITDEQVLMTP |
| 2.5.1.54/ | dehydro- | EQLKAAFPLSLQQEAQIADS |
| P00888 | 3-deoxyheptonate | RKSISDIIAGRDPRLLVVCG |
| | aldolase | PCSIHDPETALEYARRFKAL |
| | Escherichia coli | AAEVSDSLYLVMRVYFEKPR |
| | | TTVGWKGLINDPHMDGSFDV |
| | | EAGLQIARKLLLELVNMGLP |
| | | LATEALNPNSPQYLGDLFSW |
| | | SAIGARTTESQTHREMASGL |
| | | SMPVGFKNGTDGSLATAINA |
| | | MRAAAQPHRFVGINQAGQVA |
| | | LLQTQGNPDGHVILRGGKAP |
| | | NYSPADVAQCEKEMEQAGLR |
| | | PSLMVDCSHGNSNKDYRRQP |
| | | AVAESVVAQIKDGNRSIIGL |
| | | MIESNIHEGNQSSEQPRSEM |
| | | KYGVSVTDACISWEMTDALL |
| | | REIHQDLNGQLTARVA |
| | | (SEQ ID NO. 2) |
| EC: | bifunctional | MVAELTALRDQIDEVDKALL |
| 5.4.99.5 | chorismate | NLLAKRLELVAEVGEVKSRF |
| and | mutase/ | GLPIYVPEREASILASRRAE |
| EC: | prephenate | AEALGVPPDLIEDVLRRVMR |
| 1.3.1.12/ | dehydrogenase | ESYSSENDKGFKTLCPSLRP |
| P07023 | Escherichia coli | VVIVGGGGQMGRLFEKMLTL |
| | | SGYQVRILEQHDWDRAADIV |
| | | ADAGMVIVSVPIHVTEQVIG |
| | | KLPPLPKDCILVDLASVKNG |
| | | PLQAMLVAHDGPVLGLHPMF |
| | | GPDSGSLAKQVVVWCDGRKP |
| | | EAYQWFLEQIQVWGARLHRI |
| | | SAVEHDQNMAFIQALRHFAT |
| | | FAYGLHLAEENVQLEQLLAL |
| | | SSPIYRLELAMVGRLFAQDP |
| | | QLYADIIMSSERNLALIKRY |
| | | YKRFGEAIELLEQGDKQAFI |
| | | DSFRKVEHWFGDYVQRFQSE |
| | | SRVLLRQANDNRQ |
| | | (SEQ ID NO. 3) |
| EC: | UDP-Glycosyl- | MGSQIIHNSQKPHVVCVPYP |
| 2.4.1.-/ | transferase | AQGHINPMMRVAKLLHARGF |
| Q9SK82 | Arabidopsis | YVTFVNTVYNHNRFLRSRGS |
| | thaliana | NALDGLPSFRFESIADGLPE |
| | | TDMDATQDITALCESTMKNC |
| | | LAPFRELLQRINAGDNVPPV |
| | | SCIVSDGCMSFTLDVAEELG |
| | | VPEVLFWTTSGCAFLAYLHF |
| | | YLFIEKGLCPLKDESYLTKE |
| | | YLEDTVIDFIPTMKNVKLKD |
| | | IPSFIRTTNPDDVMISFALR |
| | | ETERAKRASAIILNTFDDLE |
| | | HDVVHAMQSILPPVYSVGPL |
| | | HLLANREIEEGSEIGMMSSN |
| | | LWKEEMECLDWLDTKTQNSV |
| | | IYINFGSITVLSVKQLVEFA |
| | | WGLAGSGKEFLWVIRPDLVA |
| | | GEEAMVPPDFLMETKDRSML |
| | | ASWCPQEKVLSHPAIGGFLT |
| | | HCGWNSILESLSCGVPMVCW |
| | | PFFADQQMNCKFCCDEWDVG |
| | | IEIGGDVKREEVEAVVRELM |
| | | DGEKGKKMREKAVEWQRLAE |
| | | KATEHKLGSSVMNFETVVSK |
| | | FLLGQKSQD |
| | | (SEQ ID NO. 4) |
| EC: | 4-hydroxy- | MKPEDFRASTQRPFTGEEYL |
| 1.14.14.9/ | phenylacetate | KSLQDGREIYIYGERVKDVT |
| Q57160 | 3-monooxygenase | THPAFRNAAASVAQLYDALH |
| | oxygenase | KPEMQDSLCWNTDTGSGGYT |
| | * | HKFFRVAKSADDLRQQRDAI |
| | (S210T, A211L | AEWSRLSYGWMGRTPDYKAA |
| | and Q212E) | FGCALGANPGFYGQFEQNAR |
| | Escherichia coli | NWYTRIQETGLYFNHAIVNP |
| | | PIDRHLPTDKVKDVYIKLEK |
| | | ETDAGIIVSGAKVVATNSAL |
| | | THYNMIGFGTLEVMGENPDF |
| | | ALMFVAPMDADGVKLISRAS |
| | | YEMVAGATGSPYDYPLSSRF |
| | | DENDAILVMDNVLIPWENVL |
| | | IYRDFDRCRRWTMEGGFARM |
| | | YPLQACVRLAVKLDFITALL |
| | | KKSLECTGTLEFRGVQADLG |
| | | EVVAWRNTFWALSDSMCSEA |
| | | TPWVNGAYLPDHAALQTYRV |
| | | LAPMAYAKIKNIIERNVTSG |
| | | LIYLPSSARDLNNPQIDQYL |
| | | AKYVRGSNGMDHVQRIKILK |
| | | LMWDAIGSEFGGRHELYEIN |
| | | YSGSQDEIRLQCLRQAQNSG |
| | | NMDKMMAMVDRCLSEYDQDG |
| | | WTVPHLHNNDDINMLDKLLK |
| | | (SEQ ID NO. 35) |

Gene Sequences:

TABLE 8
Listing of gene sequences.

| EC | | |
|---|---|---|
| number/ | | |
| Uniprot | Gene/ | Sequence |
| ID | Organism | (SEQ ID NO.) |
| EC: | aroF^{wt} | ATGCAAAAAGACGCGCTGAA |
| 2.5.1.54/ | Escherichia | TAACGTACATATTACCGACG |
| P00888 | coli | AACAGGTTTTAATGACTCCG |
| | | GAACAACTGAAGGCCGCTTT |
| | | TCCATTGAGCCTGCAACAAG |
| | | AAGCCCAGATTGCTGACTCG |
| | | CGTAAAAGCATTTCAGATAT |
| | | TATCGCCGGGCGCGATCCTC |
| | | GTCTGCTGGTAGTATGTGGT |
| | | CCTTGTTCCATTCATGATCC |
| | | GGAAACTGCTCTGGAATATG |
| | | CTCGTCGATTTAAAGCCCTT |
| | | GCCGCAGAGGTCAGCGATAG |
| | | CCTCTATCTGGTAATGCGCG |
| | | TCTATTTTGAAAAACCCCGT |
| | | ACCACTGTCGGCTGGAAAGG |
| | | GTTAATTAACGATCCCCATA |
| | | TGGATGGCTCTTTTGATGTA |
| | | GAAGCCGGGCTGCAGATCGC |
| | | GCGTAAATTGCTGCTTGAGC |
| | | TGGTGAATATGGGACTGCCA |
| | | CTGGCGACGGAAGCGTTAGA |
| | | TCCGAATAGCCCGCAATACC |
| | | TGGGCGATCTGTTTAGCTGG |
| | | TCAGCAATTGGTGCTCGTAC |
| | | AACGGAATCGCAAACTCACC |
| | | GTGAAATGGCCTCCGGGCTT |
| | | TCCATGCCGGTTGGTTTTAA |
| | | AAACGGCACCGACGGCAGTC |
| | | TGGCAACAGCAATTAACGCT |
| | | ATGCGCGCCGCCGCCCAGCC |
| | | GCACCGTTTTGTTGGCATTA |
| | | ACCAGGCAGGGCAGGTTGCG |
| | | TTGCTACAAACTCAGGGGAA |
| | | TCCGGACGGCCATGTGATCC |
| | | TGCGCGGTGGTAAAGCGCCG |
| | | AACTATAGCCCTGCGGATGT |
| | | TGCGCAATGTGAAAAAGAGA |
| | | TGGAACAGGCGGGACTGCGC |
| | | CCGTCTCTGATGGTAGATTG |
| | | CAGCCACGGTAATTCCAATA |
| | | AAGATTATCGCCGTCAGCCT |
| | | GCGGTGGCAGAATCCGTGGT |
| | | TGCTCAAATCAAAGATGGCA |
| | | ATCGCTCAATTATTGGTCTG |
| | | ATGATCGAAAGTAATATCCA |
| | | CGAGGGCAATCAGTCTTCCG |
| | | AGCAACCGCGCAGTGAAATG |
| | | AAATACGGTGTATCCGTAAC |
| | | CGATGCCTGCATTAGCTGGG |
| | | AAATGACCGATGCCTTGCTG |
| | | CGTGAAATTCATCAGGATCT |
| | | GAACGGGCAGCTGACGGCTC |
| | | GCGTGGCTTAA |
| | | (SEQ ID NO. 5) |
| | aroF^{fbr} | ATGCAAAAAGACGCGCTGAA |
| | (D147N) | TAACGTACATATTACCGACG |
| | Escherichia | AACAGGTTTTAATGACTCCG |
| | coli | GAACAACTGAAGGCCGCTTT |
| | | TCCATTGAGCCTGCAACAAG |
| | | AAGCCCAGATTGCTGACTCG |
| | | CGTAAAAGCATTTCAGATAT |
| | | TATCGCCGGGCGCGATCCTC |
| | | GTCTGCTGGTAGTATGTGGT |
| | | CCTTGTTCCATTCATGATCC |
| | | GGAAACTGCTCTGGAATATG |
| | | CTCGTCGATTTAAAGCCCTT |
| | | GCCGCAGAGGTCAGCGATAG |
| | | CCTCTATCTGGTAATGCGCG |
| | | TCTATTTTGAAAAACCCCGT |
| | | ACCACTGTCGGCTGGAAAGG |
| | | GTTAATTAACGATCCCCATA |
| | | TGGATGGCTCTTTTGATGTA |
| | | GAAGCCGGGCTGCAGATCGC |
| | | GCGTAAATTGCTGCTTGAGC |
| | | TGGTGAATATGGGACTGCCA |
| | | CTGGCGACGGAAGCGTTAAA |
| | | TCCGAATAGCCCGCAATACC |
| | | TGGGCGATCTGTTTAGCTGG |
| | | TCAGCAATTGGTGCTCGTAC |
| | | AACGGAATCGCAAACTCACC |
| | | GTGAAATGGCCTCCGGGCTT |
| | | TCCATGCCGGTTGGTTTTAA |
| | | AAACGGCACCGACGGCAGTC |
| | | TGGCAACAGCAATTAACGCT |
| | | ATGCGCGCCGCCGCCCAGCC |
| | | GCACCGTTTTGTTGGCATTA |
| | | ACCAGGCAGGGCAGGTTGCG |
| | | TTGCTACAAACTCAGGGGAA |
| | | TCCGGACGGCCATGTGATCC |
| | | TGCGCGGTGGTAAAGCGCCG |
| | | AACTATAGCCCTGCGGATGT |
| | | TGCGCAATGTGAAAAAGAGA |
| | | TGGAACAGGCGGGACTGCGC |
| | | CCGTCTCTGATGGTAGATTG |
| | | CAGCCACGGTAATTCCAATA |
| | | AAGATTATCGCCGTCAGCCT |
| | | GCGGTGGCAGAATCCGTGGT |
| | | TGCTCAAATCAAAGATGGCA |
| | | ATCGCTCAATTATTGGTCTG |
| | | ATGATCGAAAGTAATATCCA |
| | | CGAGGGCAATCAGTCTTCCG |
| | | AGCAACCGCGCAGTGAAATG |
| | | AAATACGGTGTATCCGTAAC |
| | | CGATGCCTGCATTAGCTGGG |
| | | AAATGACCGATGCCTTGCTG |
| | | CGTGAAATTCATCAGGATCT |
| | | GAACGGGCAGCTGACGGCTC |
| | | GCGTGGCTTAA |
| | | (SEQ ID NO. 6) |
| EC: | AtUGT85A1 | ATGGGATCTCAGATCATTCA |
| 2.4.1.-/ | Arabidopsis | TAACTCACAAAAACCACATG |
| Q9SK82 | thaliana | TAGTTTGTGTTCCATATCCG |
| | | GCTCAAGGCCACATCAACCC |
| | | TATGATGAGAGTGGCTAAAC |
| | | TCCTCCACGCCAGAGGCTTC |
| | | TACGTCACCTTCGTCAACAC |
| | | CGTCTACAACCACAATCGTT |
| | | TCCTTCGTTCTCGTGGGTCC |
| | | AATGCCCTAGATGGACTTCC |
| | | TTCGTTCCGATTTGAGTCCA |
| | | TTGCTGACGGTCTACCAGAG |
| | | ACAGACATGGATGCCACGCA |
| | | GGACATCACAGCTCTTTGCG |
| | | AGTCCACCATGAAGAACTGT |
| | | CTCGCTCCGTTCAGAGAGCT |
| | | TCTCCAGCGGATCAACGCTG |
| | | GAGATAATGTTCCTCCGGTA |
| | | AGCTGTATTGTATCTGACGG |
| | | TTGTATGAGCTTTACTCTTG |
| | | ATGTTGCGGAGGAGCTTGGA |
| | | GTCCCGGAGGTTCTTTTTTG |
| | | GACAACCAGTGGCTGTGCGT |
| | | TCCTGGCTTATCTACACTTT |
| | | TATCTCTTCATCGAGAAGGG |
| | | CTTATGTCCGCTAAAAGATG |
| | | AGAGTTACTTGACGAAGGAG |
| | | TACTTAGAAGACACGGTTAT |
| | | AGATTTTATACCAACCATGA |
| | | AGAATGTGAAACTAAAGGAT |
| | | ATTCCTAGCTTCATACGTAC |
| | | CACTAATCCTGATGATGTTA |
| | | TGATTAGTTTCGCCCTCCGC |
| | | GAGACCGAGCGAGCCAAACG |
| | | TGCTTCTGCTATCATTCTAA |
| | | ACACATTTGATGACCTTGAG |
| | | CATGATGTTGTTCATGCTAT |
| | | GCAATCTATCTTACCTCCGG |
| | | TTTATTCAGTTGGACCGCTT |
| | | CATCTCTTAGCAAACCGGGA |
| | | GATTGAAGAAGGTAGTGAGA |
| | | TTGGAATGATGAGTTCGAAT |
| | | TTATGGAAAGAGGAGATGGA |
| | | GTGTTTGGATTGGCTTGATA |
| | | CTAAGACTCAAAATAGTGTC |
| | | ATTTATATCAACTTTGGGAG |
| | | CATAACGGTTTTGAGTGTGA |
| | | AGCAGCTTGTGGAGTTTGCT |
| | | TGGGGTTTGGGGGGAAGTGG |
| | | GAAAGAGTTTTTATGGGTGA |
| | | TCCGGCCAGATTTAGTAGCG |
| | | GGAGAGGAGGCTATGGTTCC |
| | | GCCGGACTTTTTAATGGAGA |
| | | CTAAAGACCGCAGTATGCTA |
| | | GCGAGTTGGTGTCCTCAAGA |
| | | GAAAGTACTTTCTCATCCTG |
| | | CTATTGGAGGGTTTTTGACG |
| | | CATTGCGGGTGGAACTCGAT |
| | | ATTGGAAAGTCTTTCGTGTG |
| | | GAGTTCCGATGGTGTGTTGG |
| | | CCATTTTTTGCTGACCAGCA |
| | | AATGAATTGTAAGTTTTGTT |
| | | GTGACGAGTGGGATGTTGGG |
| | | ATTGAGATAGGTGGAGATGT |
| | | GAAGAGAGAGGAAGTTGAGG |
| | | CGGTGGTTAGAGAGCTCATG |
| | | GATGGAGAGAAGGGAAAGAA |
| | | AATGAGAGAAAAGGCGGTAG |
| | | AGTGGCAGCGCTTAGCCGAG |
| | | AAAGCGACGGAACATAAACT |
| | | TGGTTCTTCCGTTATGAATT |
| | | TTGAGACGGTTGTTAGCAAG |
| | | TTTCTTTTGGGACAAAAATC |
| | | ACAGGATTAA |
| | | (SEQ ID NO. 7) |
| | AtUGT85A1 | ATGGGATCACAGATCATACA |
| | optimized | CAACTCGCAGAAACCCCATG |
| | | TGGTCTGTGTACCGTACCCC |
| | | GCTCAGGGCCATATTAATCC |
| | | AATGATGCGTGTCGCTAAAT |
| | | TACTTCATGCCAGAGGGTTT |
| | | TATGTAACATTCGTCAATAC |
| | | AGTGTATAATCACAATAGAT |
| | | TTCTTAGAAGCCGCGGGTCG |
| | | AATGCGTTAGACGGCCTGCC |
| | | CTCCTTCCGGTTTGAGTCAA |
| | | TAGCCGACGGGTTGCCTGAA |
| | | ACGGATATGGACGCCACACA |
| | | GGACATAACGGCTCTGTGTG |
| | | AGTCGACTATGAAGAATTGT |
| | | CTGGCTCCCTTCCGCGAGTT |
| | | GCTGCAACGGATAAATGCTG |
| | | GGGATAACGTACCTCCTGTT |
| | | AGCTGTATAGTATCCGATGG |
| | | GTGCATGTCCTTTACCCTTG |
| | | ATGTAGCAGAGGAACTTGGA |
| | | GTGCCGGAAGTTTTGTTCTG |
| | | GACCACTAGCGGGTGTGCCT |
| | | TTTTAGCATACCTGCACTTT |
| | | TATTTATTTATAGAAAAGGG |
| | | GTTGTGTCCCTTAAAAGACG |
| | | AGTCCTATTTGACGAAGGAA |
| | | TACCTTGAGGACACGGTTAT |
| | | AGATTTCATACCGACTATGA |
| | | AAAACGTTAAGCTGAAGGAT |
| | | ATACCTAGCTTCATACGCAC |
| | | AACTAATCCGGATGATGTTA |
| | | TGATCTCTTTTGCCCTGCGT |
| | | GAGACAGAGCGCGCTAAGCG |
| | | GGCGTCTGCGATTATATTGA |
| | | ACACATTTGACGATCTGGAA |
| | | CACGATGTTGTCCACGCAAT |
| | | GCAGTCCATTCTTCCTCCGG |
| | | TATATTCAGTGGGACCCTTG |
| | | CACCTTTTAGCGAACCGGGA |
| | | AATCGAAGAAGGATCTGAAA |
| | | TAGGTATGATGTCTTCCAAC |
| | | TTATGGAAGGAAGAAATGGA |
| | | GTGTCTTGACTGGTTGGATA |
| | | CAAAGACACAAAATTCCGTA |
| | | ATATACATAAACTTCGGCAG |
| | | CATCACGGTGTTGAGCGTAA |
| | | AACAGCTGGTCGAATTCGCT |
| | | TGGGGTTTGGCAGGTTCCGG |
| | | TAAGGAGTTCTTGTGGGTCA |
| | | TAAGACCAGACTTAGTCGCG |
| | | GGGGAAGAAGCAATGGTACC |
| | | CCCCGACTTCCTTATGGAGA |
| | | CGAAAGACCGTTCCATGTTG |
| | | GCCTCTTGGTGCCCTCAAGA |
| | | GAAAGTCTTGTCACATCCCG |
| | | CTATTGGAGGGTTCCTGACA |
| | | CACTGTGGTTGGAATTCAAT |
| | | TCTTGAGAGCTTATCGTGTG |
| | | GGGTGCCAATGGTGTGCTGG |
| | | CCGTTCTTTGCAGATCAGCA |
| | | AATGAACTGTAAGTTTTGCT |
| | | GCGACGAATGGGATGTAGGT |
| | | ATAGAGATCGGCGGCGACGT |
| | | TAAGCGCGAGGAGGTCGAGG |
| | | CAGTTGTAAGAGAGCTGATG |
| | | GACGGTGAGAAAGGCAAAAA |
| | | AATGAGAGAAAAAGCGGTCG |
| | | AGTGGCAGCGGTTGGCTGAG |
| | | AAAGCTACGGAACATAAACT |
| | | TGGCAGTAGCGTTATGAACT |
| | | TTGAAACTGTTGTATCGAAA |
| | | TTTTTGCTGGGGCAGAAAAG |
| | | CCAGGACTAA |
| | | (SEQ ID NO. 8) |
| EC: | ARO10^{wt} | ATGGCACCTGTTACAATTGA |
| 4.1.1.80/ | Saccharomyces | AAAGTTCGTAAATCAA |
| Q06408 | cerevisiae | GAAGAACGACACCTTGTTTC |
| | | CAACCGATCAGCAAC |
| | | AATTCCGTTTGGTGAATACA |
| | | TATTTAAAAGATTGTT |
| | | GTCCATCGATACGAAATCAG |
| | | TTTTCGGTGTTCCTGGTGAC |
| | | TTCAACTTATCTCTATTAGA |
| | | ATATCTCTATTCACCTAGTG |
| | | TTGAATCAGCTGGCCTAAGA |
| | | TGGGTCGGCACGTGTAATGA |
| | | ACTGAACGCCGCTTATGCGG |
| | | CCGACGGATATTCCCGTTAC |
| | | TCTAATAAGATTGGCTGTTT |
| | | AATAACCACGTATGGCGTTG |
| | | GTGAATTAAGCGCCTTGAAC |
| | | GGTATAGCCGGTTCGTTCGC |
| | | TGAAAATGTCAAAGTTTTGC |
| | | ACATTGTTGGTGTGGCCAAG |
| | | TCCATAGATTCGCGTTCAAG |
| | | TAACTTTAGTGATCGGAACC |
| | | TACATCATTTGGTCCCACAG |
| | | CTACATGATTCAAATTTTAA |
| | | AGGGCCAAATCATAAAGTAT |
| | | ATCATGATATGGTAAAAGAT |
| | | AGAGTCGCTTGCTCGGTAGC |
| | | CTACTTGGAGGATATTGAAA |
| | | CTGCATGTGACCAAGTCGAT |
| | | AATGTTATCCGCGATATTTA |
| | | CAAGTATTCTAAACCTGGTT |
| | | ATATTTTTGTTCCTGCAGAT |
| | | TTTGCGGATATGTCTGTTAC |
| | | ATGTGATAATTTGGTTAATG |
| | | TTCCACGTATATCTCAACAA |
| | | GATTGTATAGTATACCCTTC |
| | | TGAAAACCAATTGTCTGACA |
| | | TAATCAACAAGATTACTAGT |
| | | TGGATATATTCCAGTAAAAC |
| | | ACCTGCGATCCTTGGAGACG |
| | | TACTGACTGATAGGTATGGT |
| | | GTGAGTAACTTTTTGAACAA |
| | | GCTTATCTGCAAAACTGGGA |
| | | TTTGGAATTTTTCCACTGTT |
| | | ATGGGAAAATCTGTAATTGA |
| | | TGAGTCAAACCCAACTTATA |
| | | TGGGTCAATATAATGGTAAA |
| | | GAAGGTTTAAAACAAGTCTA |
| | | TGAACATTTTGAACTGTGCG |
| | | ACTTGGTCTTGCATTTTGGA |
| | | GTCGACATCAATGAAATTAA |
| | | TAATGGGCATTATACTTTTA |
| | | CTTATAAACCAAATGCTAAA |
| | | ATCATTCAATTTCATCCGAA |
| | | TTATATTCGCCTTGTGGACA |
| | | CTAGGCAGGGCAATGAGCAA |
| | | ATGTTCAAAGGAATCAATTT |
| | | TGCCCCTATTTTAAAAGAAC |
| | | TATACAAGCGCATTGACGTT |
| | | TCTAAACTTTCTTTGCAATA |
| | | TGATTCAAATGTAACTCAAT |
| | | ATACGAACGAAACAATGCGG |
| | | TTAGAAGATCCTACCAATGG |
| | | ACAATCAAGCATTATTACAC |
| | | AAGTTCACTTACAAAAGACG |
| | | ATGCCTAAATTTTTGAACCC |
| | | TGGTGATGTTGTCGTTTGTG |
| | | AAACAGGCTCTTTTCAATTC |
| | | TCTGTTCGTGATTTCGCGTT |
| | | TCCTTCGCAATTAAAATATA |
| | | TATCGCAAGGATTTTTCCTT |
| | | TCCATTGGCATGGCCCTTCC |
| | | TGCCGCCCTAGGTGTTGGAA |
| | | TTGCCATGCAAGACCACTCA |
| | | AACGCTCACATCAATGGTGG |
| | | CAACGTAAAAGAGGACTATA |
| | | AGCCAAGATTAATTTTGTTT |
| | | GAAGGTGACGGTGCAGCACA |
| | | GATGACAATCCAAGAACTGA |
| | | GCACCATTCTGAAGTGCAAT |
| | | ATTCCACTAGAAGTTATCAT |
| | | TTGGAACAATAACGGCTACA |
| | | CTATTGAAAGAGCCATCATG |
| | | GGCCCTACCAGGTCGTATAA |
| | | CGACGTTATGTCTTGGAAAT |
| | | GGACCAAACTATTTGAAGCA |
| | | TTCGGAGACTTCGACGGAAA |
| | | GTATACTAATAGCACTCTCA |
| | | TTCAATGTCCCTCTAAATTA |
| | | GCACTGAAATTGGAGGAGCT |
| | | TAAGAATTCAAACAAAAGAA |
| | | GCGGGATAGAACTTTTAGAA |
| | | GTCAAATTAGGCGAATTGGA |
| | | TTTCCCCGAACAGCTAAAGT |
| | | GCATGGTTGAAGCAGCGGCA |
| | | CTTAAAAGAAATAAAAAATA |
| | | G |
| | | (SEQ ID NO. 9) |
| | ScARO10* | ATGGCTCCGGTTACCATCGA |
| | optimized | AAAATTCGTTAACCAGGAAG |
| | | AACGTCACCTGGTTTCTAAC |
| | | CGTTCTGCTACCATCCCGTT |
| | | CGGTGAATACATCTTCAAAC |
| | | GTCTGCTGTCTATCGACACC |
| | | AAATCTGTTTTCGGTGTTCC |
| | | GGGTGACTTCAACCTGTCTC |
| | | TGCTGGAATACCTGTACTCT |
| | | CCGTCTGTTGAATCTGCTGG |
| | | TCTGCGTTGGGTTGGTACCT |
| | | GCAACGAACTGAACGCTGCT |
| | | TACGCTGCTGACGGTTACTC |
| | | TCGTTACTCTAACAAAATCG |
| | | GTTGCCTGATCACCACCTAC |
| | | GGTGTTGGTGAACTGTCTGC |
| | | TCTGAACGGTATCGCTGGTT |
| | | CTTTCGCTGAAAACGTTAAA |
| | | GTTCTGCACATCGTTGGTGT |
| | | TGCTAAATCTATCGACTCTC |
| | | GTTCTTCTAACTTCTCTGAC |
| | | CGTAACCTGCACCACCTGGT |
| | | TCCGCAGCTGCACGACTCTA |
| | | ACTTCAAAGGTCCGAACCAC |
| | | AAAGTTTACCACGACATGGT |
| | | TAAAGACCGTGTTGCTTGCT |
| | | CTGTTGCTTACCTGGAAGAC |
| | | ATCGAAACCGCTTGCGACCA |
| | | GGTTGACAACGTTATCCGTG |
| | | ACATCTACAAATACTCTAAA |
| | | CCGGGTTACATCTTCGTTCC |
| | | GGCTGACTTCGCTGACATGT |
| | | CTGTTACCTGCGACAACCTG |
| | | GTTAACGTTCCGCGTATCTC |
| | | TCAGCAGGACTGCATCGTTT |
| | | ACCCGTCTGAAAACCAGCTG |
| | | TCTGACATCATCAACAAAAT |
| | | CACCTCTTGGATCTACTCTT |
| | | CTAAAACCCCGGCTATCCTG |
| | | GGTGACGTTTTAACCGACCG |
| | | TTACGGTGTAAGCAACTTCC |
| | | TGAACAAACTGATCTGCAAA |
| | | ACCGGTATCTGGAACTTCTC |
| | | TACCGTTATGGGTAAATCTG |
| | | TTATCGACGAATCTAACCCG |
| | | ACCTACATGGGTCAGTACAA |
| | | CGGTAAAGAAGGTCTGAAAC |
| | | AGGTTTACGAACACTTCGAA |
| | | CTGTGCGACCTGGTTCTGCA |
| | | CTTCGGTGTTGACATCAACG |
| | | AAATCAACAACGGTCACTAC |
| | | ACCTTCACCTACAAACCGAA |
| | | CGCTAAAATCATCCAGTTCC |
| | | ACCCGAACTACATCCGTCTG |
| | | GTTGACACCCGTCAGGGTAA |
| | | CGAACAGATGTTCAAAGGTA |
| | | TCAACTTCGCTCCGATCCTG |
| | | AAAGAACTGTACAAACGTAT |
| | | CGACGTTTCTAAACTGTCTC |
| | | TGCAGTACGACTCTAACGTT |
| | | ACCCAGTACACCAACGAAAC |
| | | CATGCGTCTGGAAGACCCGA |
| | | CCAACGGTCAGTCTTCTATC |
| | | ATCACCCAGGTTCACCTGCA |
| | | GAAAACCATGCCGAAATTCC |
| | | TGAACCCGGGTGACGTTGTT |
| | | GTTTGCGAAACCGGTTCTTT |
| | | CCAGTTCTCTGTTCGTGACT |
| | | TCGCTTTCCCGTCTCAGCTG |
| | | AAATACATCTCTCAGGGTTT |
| | | CTTCCTGTCTATCGGTATGG |
| | | CTCTGCCGGCTGCTCTGGGT |
| | | GTTGGTATCGCTATGCAGGA |
| | | CCACTCTAACGCTCACATCA |
| | | ACGGTGGTAACGTTAAAGAA |
| | | GACTACAAACCGCGTCTGAT |
| | | CCTGTTCGAAGGTGACGGTG |
| | | CTGCTCAGATGACCATCCAG |
| | | GAACTGTCTACCATCCTGAA |
| | | ATGCAACATCCCGCTGGAAG |
| | | TTATCATCTGGAACAACAAC |
| | | GGTTACACCATCGAACGTGC |
| | | TATCATGGGTCCGACCCGTT |
| | | CTTACAACGACGTTATGTCT |
| | | TGGAAATGGACCAAACTGTT |
| | | CGAAGCGTTCGGTGACTTCG |
| | | ACGGTAAATACACCAACTCT |
| | | ACCCTGATCCAGTGCCCGTC |
| | | TAAACTGGCTCTGAAACTGG |
| | | AAGAACTGAAAAACTCTAAC |
| | | AAACGTTCTGGTATCGAACT |
| | | GCTGGAAGTTAAACTGGGTG |
| | | AACTGGACTTCCCGGAACAG |
| | | CTGAAATGCATGGTTGAAGC |
| | | TGCTGCTCTGAAACGTAACA |
| | | AAAAATAAAAGCTTTAA |
| | | (SEQ ID NO. 10) |
| EC: | tyrA^{wt} | ATGGTTGCTGAATTGACCGC |
| 5.4.99.5 | Escherichia | ATTACGCGATCAAATTGATG |
| and | coli | AAGTCGATAAAGCGCTGCTG |
| EC: | | AATTTATTAGCGAAGCGTCT |
| 1.3.1.12/ | | GGAACTGGTTGCTGAAGTGG |
| P07023 | | GCGAGGTGAAAAGCCGCTTT |
| | | GGACTGCCTATTTATGTTCC |
| | | GGAGCGCGAGGCATCTATGT |
| | | TGGCCTCGCGTCGTGCAGAG |
| | | GCGGAAGCTCTGGGTGTACC |
| | | GCCAGATCTGATTGAGGATG |
| | | TTTTGCGTCGGGTGATGCGT |
| | | GAATCTTACTCCAGTGAAAA |
| | | CGACAAAGGATTTAAAACAC |
| | | TTTGTCCGTCACTGCGTCCG |
| | | GTGGTTATCGTCGGCGGTGG |
| | | CGGTCAGATGGGACGCCTGT |
| | | TCGAGAAGATGCTGACCCTC |
| | | TCGGGTTATCAGGTGCGGAT |
| | | TCTGGAGCAACATGACTGGG |
| | | ATCGAGCGGCTGATATTGTT |
| | | GCCGATGCCGGAATGGTGAT |
| | | TGTTAGTGTGCCAATCCACG |
| | | TTACTGAGCAAGTTATTGGC |
| | | AAATTACCGCCTTTACCGAA |
| | | AGATTGTATTCTGGTCGATC |
| | | TGGCATCAGTGAAAAATGGG |
| | | CCATTACAGGCCATGCTGGT |
| | | GGCGCATGATGGTCCGGTGC |
| | | TGGGGCTACACCCGATGTTC |
| | | GGTCCGGACAGCGGTAGCCT |
| | | GGCAAAGCAAGTTGTGGTCT |
| | | GGTGTGATGGACGTAAACCG |
| | | GAAGCATACCAATGGTTTCT |
| | | GGAGCAAATTCAGGTCTGGG |
| | | GCGCTCGGCTGCATCGTATT |
| | | AGCGCCGTCGAGCACGATCA |
| | | GAATATGGCGTTTATTCAGG |
| | | CACTGCGCCACTTTGCTACT |
| | | TTTGCTTACGGGCTGCACCT |
| | | GGCAGAAGAAAATGTTCAGC |
| | | TTGAGCAACTTCTGGCGCTC |
| | | TCTTCGCCGATTTACCGCCT |
| | | TGAGCTGGCGATGGTCGGGC |
| | | GACTGTTTGCTCAGGATCCG |
| | | CAGCTTTATGCCGACATCAT |
| | | TATGTCGTCAGAGCGTAATC |
| | | TGGCGTTAATCAAACGTTAC |
| | | TATAAGCGTTTCGGCGAGGC |
| | | GATTGAGTTGCTGGAGCAGG |
| | | GCGATAAGCAGGCGTTTATT |
| | | GACAGTTTCCGCAAGGTGGA |
| | | GCACTGGTTCGGCGATTACG |
| | | CACAGCGTTTTCAGAGTGAA |
| | | AGCCGCGTGTTATTGCGTCA |
| | | GGCGAATGACAATCGCCAGT |
| | | AA |
| | | (SEQ ID NO. 11) |
| | TyrA^{fbr} | ATGGTTGCTGAATTGACCGC |
| | (M531 and | ATTACGCGATCAAATTGATG |
| | A354V) | AAGTCGATAAAGCGCTGCTG |
| | Escherichia | AATTTATTAGCGAAGCGTCT |
| | coli | GGAACTGGTTGCTGAAGTGG |
| | | GCGAGGTGAAAAGCCGCTTT |
| | | GGACTGCCTATTTATGTTCC |
| | | GGAGCGCGAGGCATCTATCT |
| | | TGGCCTCGCGTCGTGCAGAG |
| | | GCGGAAGCTCTGGGTGTACC |
| | | GCCAGATCTGATTGAGGATG |
| | | TTTTGCGTCGGGTGATGCGT |
| | | GAATCTTACTCCAGTGAAAA |
| | | CGACAAAGGATTTAAAACAC |
| | | TTTGTCCGTCACTGCGTCCG |
| | | GTGGTTATCGTCGGCGGTGG |
| | | CGGTCAGATGGGACGCCTGT |
| | | TCGAGAAGATGCTGACCCTC |
| | | TCGGGTTATCAGGTGCGGAT |
| | | TCTGGAGCAACATGACTGGG |
| | | ATCGAGCGGCTGATATTGTT |
| | | GCCGATGCCGGAATGGTGAT |
| | | TGTTAGTGTGCCAATCCACG |
| | | TTACTGAGCAAGTTATTGGC |
| | | AAATTACCGCCTTTACCGAA |
| | | AGATTGTATTCTGGTCGATC |
| | | TGGCATCAGTGAAAAATGGG |
| | | CCATTACAGGCCATGCTGGT |
| | | GGCGCATGATGGTCCGGTGC |
| | | TGGGGCTACACCCGATGTTC |
| | | GGTCCGGACAGCGGTAGCCT |
| | | GGCAAAGCAAGTTGTGGTCT |
| | | GGTGTGATGGACGTAAACCG |
| | | GAAGCATACCAATGGTTTCT |
| | | GGAGCAAATTCAGGTCTGGG |
| | | GCGCTCGGCTGCATCGTATT |
| | | AGCGCCGTCGAGCACGATCA |
| | | GAATATGGCGTTTATTCAGG |
| | | CACTGCGCCACTTTGCTACT |
| | | TTTGCTTACGGGCTGCACCT |
| | | GGCAGAAGAAAATGTTCAGC |
| | | TTGAGCAACTTCTGGCGCTC |
| | | TCTTCGCCGATTTACCGCCT |
| | | TGAGCTGGCGATGGTCGGGC |
| | | GACTGTTTGCTCAGGATCCG |
| | | CAGCTTTATGCCGACATCAT |
| | | TATGTCGTCAGAGCGTAATC |
| | | TGGCGTTAATCAAACGTTAC |
| | | TATAAGCGTTTCGGCGAGGC |
| | | GATTGAGTTGCTGGAGCAGG |
| | | GCGATAAGCAGGCGTTTATT |
| | | GACAGTTTCCGCAAGGTGGA |
| | | GCACTGGTTCGGCGATTACG |
| | | TACAGCGTTTTCAGAGTGAA |
| | | AGCCGCGTGTTATTGCGTCA |
| | | GGCGAATGACAATCGCCAGT |
| | | AA |
| | | (SEQ ID NO. 12) |
| EC: | hpaB | ATGAAACCAGAAGATTTCCG |
| 1.14.14.9/ | wild-type | CGCCAGTACCCAACGTCCTT |
| Q57160 | Escherichia | TCACCGGGGAAGAGTATCTG |
| | coli | AAAAGCCTGCAGGATGGTCG |
| | | CGAGATCTATATCTATGGCG |
| | | AGCGAGTGAAAGACGTCACC |
| | | ACTCATCCGGCATTTCGTAA |
| | | TGCGGCAGCGTCTGTTGCCC |
| | | AGCTGTACGACGCACTGCAC |
| | | AAACCGGAGATGCAGGACTC |
| | | TCTGTGTTGGAACACCGACA |
| | | CCGGCAGCGGCGGCTATACC |
| | | CATAAATTCTTCCGCGTGGC |
| | | GAAAAGTGCCGACGACCTGC |
| | | GCCAGCAACGCGACGCCATC |
| | | GCTGAGTGGTCACGCCTGAG |
| | | CTATGGCTGGATGGGCCGTA |
| | | CCCCAGACTACAAAGCCGCT |
| | | TTCGGTTGCGCACTGGGCGC |
| | | GAATCCGGGCTTTTACGGTC |
| | | AGTTCGAGCAGAACGCCCGT |
| | | AACTGGTACACCCGTATTCA |
| | | GGAAACTGGCCTCTACTTTA |
| | | ACCACGCGATTGTTAACCCA |
| | | CCGATCGATCGTCATTTGCC |
| | | GACCGATAAAGTGAAAGACG |
| | | TTTACATCAAGCTGGAAAAA |
| | | GAGACTGACGCCGGGATTAT |
| | | CGTCAGCGGTGCGAAAGTGG |
| | | TTGCCACCAACTCGGCGCTG |
| | | ACTCACTACAACATGATTGG |
| | | CTTCGGCTCGGCACAAGTGA |
| | | TGGGCGAAAACCCGGACTTC |
| | | GCACTGATGTTCGTTGCGCC |
| | | AATGGATGCCGATGGCGTGA |
| | | AATTAATCTCCCGCGCCTCT |
| | | TATGAGATGGTCGCGGGTGC |
| | | TACCGGCTCGCCATACGACT |
| | | ACCCGCTCTCCAGCCGCTTC |
| | | GATGAGAACGATGCGATTCT |
| | | GGTGATGGATAACGTGCTGA |
| | | TTCCATGGGAAAACGTGCTG |
| | | ATCTACCGCGATTTTGATCG |
| | | CTGCCGTCGCTGGACGATGG |
| | | AAGGCGGTTTTGCCCGTATG |
| | | TATCCGCTGCAAGCCTGTGT |
| | | GCGCCTGGCAGTGAAATTAG |
| | | ACTTCATTACGGCACTGCTG |
| | | AAAAAATCACTCGAATGTAC |
| | | CGGCACCCTGGAGTTCCGTG |
| | | GTGTGCAGGCCGATCTCGGT |
| | | GAAGTGGTAGCGTGGCGCAA |
| | | CACCTTCTGGGCATTGAGTG |
| | | ACTCGATGTGTTCAGAAGCA |
| | | ACGCCGTGGGTCAACGGGGC |
| | | TTATTTACCGGATCATGCCG |
| | | CACTGCAAACCTATCGCGTA |
| | | CTGGCACCAATGGCCTACGC |
| | | GAAGATCAAAAACATTATCG |
| | | AACGCAACGTTACCAGTGGC |
| | | CTGATCTATCTCCCTTCCAG |
| | | TGCCCGTGACCTGAATAATC |
| | | CGCAGATCGACCAGTATCTG |
| | | GCGAAGTATGTGCGCGGTTC |
| | | GAACGGTATGGATCACGTCC |
| | | AGCGCATCAAGATCCTCAAA |
| | | CTGATGTGGGATGCTATTGG |
| | | CAGCGAATTTGGTGGTCGTC |
| | | ACGAACTGTATGAAATCAAC |
| | | TACTCCGGTAGCCAGGATGA |
| | | GATTCGCCTGCAGTGTCTGC |
| | | GCCAGGCACAAAACTCCGGC |
| | | AATATGGACAAGATGATGGC |
| | | GATGGTTGATCGCTGCCTGT |
| | | CGGAATACGACCAGGACGGC |
| | | TGGACTGTGCCGCACCTGCA |
| | | CAACAACGACGATATCAACA |
| | | TGCTGGATAAGCTGCTGAAA |
| | | TAA |
| | | (SEQ ID NO. 36) |
| | hpaB* | ATGAAACCAGAAGATTTCCG |
| | (S210T, A211L | CGCCAGTACCCAACGTCCTT |
| | and Q212E) | TCACCGGGGAAGAGTATCTG |
| | Escherichia | AAAAGCCTGCAGGATGGTCG |
| | coli | CGAGATCTATATCTATGGCG |
| | | AGCGAGTGAAAGACGTCACC |
| | | ACTCATCCGGCATTTCGTAA |
| | | TGCGGCAGCGTCTGTTGCCC |
| | | AGCTGTACGACGCACTGCAC |
| | | AAACCGGAGATGCAGGACTC |
| | | TCTGTGTTGGAACACCGACA |
| | | CCGGCAGCGGCGGCTATACC |
| | | CATAAATTCTTCCGCGTGGC |
| | | GAAAAGTGCCGACGACCTGC |
| | | GCCAGCAACGCGACGCCATC |
| | | GCTGAGTGGTCACGCCTGAG |
| | | CTATGGCTGGATGGGCCGTA |
| | | CCCCAGACTACAAAGCCGCT |
| | | TTCGGTTGCGCACTGGGCGC |
| | | GAATCCGGGCTTTTACGGTC |
| | | AGTTCGAGCAGAACGCCCGT |
| | | AACTGGTACACCCGTATTCA |
| | | GGAAACTGGCCTCTACTTTA |
| | | ACCACGCGATTGTTAACCCA |
| | | CCGATCGATCGTCATTTGCC |
| | | GACCGATAAAGTGAAAGACG |
| | | TTTACATCAAGCTGGAAAAA |
| | | GAGACTGACGCCGGGATTAT |
| | | CGTCAGCGGTGCGAAAGTGG |
| | | TTGCCACCAACTCGGCGCTG |
| | | ACTCACTACAACATGATTGG |
| | | CTTCGGCACCCTGGAAGTGA |
| | | TGGGCGAAAACCCGGACTTC |
| | | GCACTGATGTTCGTTGCGCC |
| | | AATGGATGCCGATGGCGTGA |
| | | AATTAATCTCCCGCGCCTCT |
| | | TATGAGATGGTCGCGGGTGC |
| | | TACCGGCTCGCCATACGACT |
| | | ACCCGCTCTCCAGCCGCTTC |
| | | GATGAGAACGATGCGATTCT |
| | | GGTGATGGATAACGTGCTGA |
| | | TTCCATGGGAAAACGTGCTG |
| | | ATCTACCGCGATTTTGATCG |
| | | CTGCCGTCGCTGGACGATGG |
| | | AAGGCGGTTTTGCCCGTATG |
| | | TATCCGCTGCAAGCCTGTGT |
| | | GCGCCTGGCAGTGAAATTAG |
| | | ACTTCATTACGGCACTGCTG |
| | | AAAAAATCACTCGAATGTAC |
| | | CGGCACCCTGGAGTTCCGTG |
| | | GTGTGCAGGCCGATCTCGGT |
| | | GAAGTGGTAGCGTGGCGCAA |
| | | CACCTTCTGGGCATTGAGTG |
| | | ACTCGATGTGTTCAGAAGCA |
| | | ACGCCGTGGGTCAACGGGGC |
| | | TTATTTACCGGATCATGCCG |
| | | CACTGCAAACCTATCGCGTA |
| | | CTGGCACCAATGGCCTACGC |
| | | GAAGATCAAAAACATTATCG |
| | | AACGCAACGTTACCAGTGGC |
| | | CTGATCTATCTCCCTTCCAG |
| | | TGCCCGTGACCTGAATAATC |
| | | CGCAGATCGACCAGTATCTG |
| | | GCGAAGTATGTGCGCGGTTC |
| | | GAACGGTATGGATCACGTCC |
| | | AGCGCATCAAGATCCTCAAA |
| | | CTGATGTGGGATGCTATTGG |
| | | CAGCGAATTTGGTGGTCGTC |
| | | ACGAACTGTATGAAATCAAC |
| | | TACTCCGGTAGCCAGGATGA |
| | | GATTCGCCTGCAGTGTCTGC |
| | | GCCAGGCACAAAACTCCGGC |
| | | AATATGGACAAGATGATGGC |
| | | GATGGTTGATCGCTGCCTGT |
| | | CGGAATACGACCAGGACGGC |
| | | TGGACTGTGCCGCACCTGCA |
| | | CAACAACGACGATATCAACA |
| | | TGCTGGATAAGCTGCTGAAA |
| | | TAA(SEQ ID NO. 37) |

Promoter Sequence

TABLE 9
Listing of promoter sequences

| | Sequence |
|---|---|
| Type | (SEQ ID NO.) |
| T7 promoter | TAATACGACTCACTATAG |
| | (SEQ ID NO. 31) |
| lac promoter | TTTACACTTTATGCTTCCGGCTCGTATGTTG |
| | (SEQ ID NO. 32) |
| tac promoter | TTGACAATTAATCATCGGCTCGTATAATG |
| | (SEQ ID NO. 33) |
| trc promoter | TTGACAATTAATCATCCGGCTCGTATAATG |
| | (SEQ ID NO. 34) |

### EXAMPLES

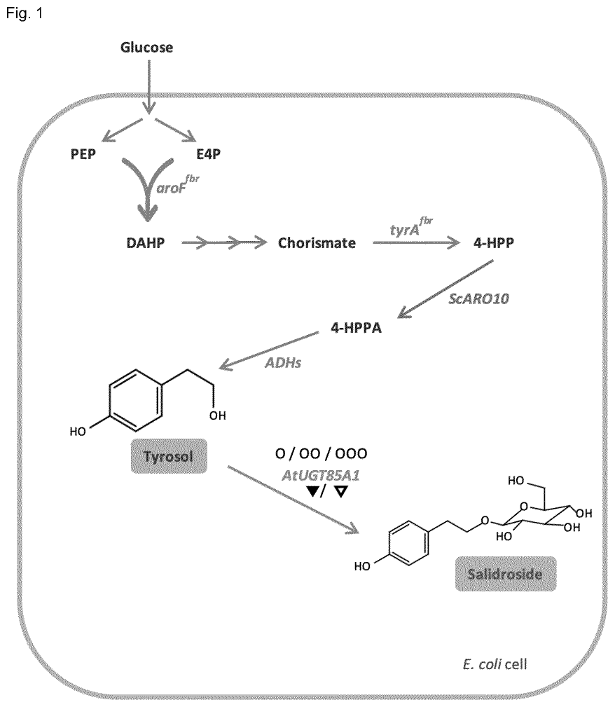

The main goal of this study was the optimization of the bioprocess of production of tyrosol and its derivatives in E. coli to titers of gram per liter, since these compounds have high-added value and important biological activities and applications. To do so, E. coli BL21 (DE3) was engineered to produce tyrosol and salidroside through the pathway depicted in FIG. 1.

### Example 1: Implementation of a Tyrosol Biosynthesis Pathway in E. coli BL21 (DE3)

The tyrosol biosynthesis pathway implemented in E. coli BL21 (DE3) (FIG. 1) begins with glucose that was converted to 4-hydroxyphenylpyruvate after several steps and finally ends with the conversion of 4-hydroxyphenylpyruvate to tyrosol by phenylpyruvate decarboxylase from S. cerevisiae (ARO10*) and endogenous alcohols dehydrogenases. Firstly, gene ARO10* from S. cerevisiae was selected and inserted into pET-21a(+) and the resulting plasmid was cloned into E. coli BL21 (DE3) to form the strain ST53. The strain ST53 produces 0.05±0.00 g/L of tyrosol after 48 h of induction with 1 mM of iPTG in M9Y medium. This result corroborated that overexpression of ScARO10 combined with endogenous ADHs could convert 4-hydroxyphenylpyruvate into tyrosol using glucose as substrate. In order to improve tyrosol production phospho-2-dehydro-3-deoxyheptonate aldolase (aroF^{fbr}) and prephenate dehydrogenase (tyrA^{fbr}) from E. coli were inserted into pET-21a(+) or pET-28a(+) and overexpressed in E. coli BL21 (DE3), obtaining the strains ST93 and ST96, respectively. These two strains were constructed to understand if these three genes work better in an operon like system or in a promoter-gene organization. With strains ST93 and 96 the tyrosol production was significantly enhanced (p<0.001), achieving 0.21±0.01 g/L for strain ST93 and 0.14±0.00 g/L for strain ST96 after 48 h of induction with 1 mM of IPTG in M9Y medium. Moreover, it was possible to verify that the production of tyrosol is inversely correlated with cell density (OD_{600 nm}), indicating that tyrosol production impacts cell growth. These results also show that tyrosol production was favoured by the heterologous expression of ScARO10* and the overexpression of aroF^{fbr} and tyrA^{fbr} in the same vector, this means that an operon like system is the best architecture to express these genes.

### Example 2: Optimization of IPTG Concentration

The isopropyl-β-d-thiogalactopyranoside (IPTG) is an effective inducer of the powerful T7 and trc promoters and is commonly used in cloning procedures. To select the best IPTG concentration to induce tyrosol production strains, the strain ST93 was induced with 0.1 and 1 mM of IPTG in M9Y medium for 48 h. Under these conditions, the strain ST93 obtained 0.65±0.07 g/L and 0.21±0.01 g/L of tyrosol after induction with 0.1 and 1 mM of IPTG, respectively (Table 10). In that way, 0.1 mM of IPTG revealed to be the best concentration to induce tyrosol production strains.

TABLE 10
Tyrosol titer (g/L) obtained with strain ST93 after induction
with 0.1 and 1 mM of IPTG in M9Y medium. Cultures were sampled
after 48 h of growth for tyrosol detection. The experiments
were independently conducted three times and experimental
data is represented by the mean ± standard deviation.

| IPTG | Time after | | Tyrosol |
|---|---|---|---|
| concentration (mM) | induction (h) | OD_{600 nm} | titer (g/L) |
| 0.1 | 48 | 3.08 ± 0.28 | 0.65 ± 0.07 |
| 1 | 48 | 2.77 ± 0.06 | 0.21 ± 0.01 |

### Example 3: Selection of the Best Phenylpyruvate Decarboxylase

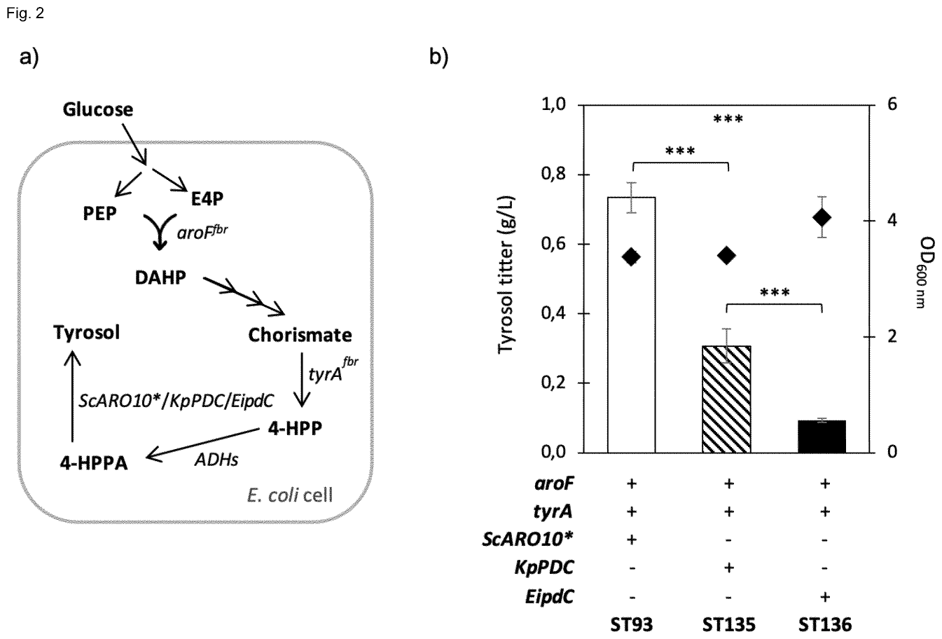

Phenylpyruvate decarboxylase is an enzyme involved in the Ehrlich pathway and catalyses the decarboxylation of phenylpyruvate to phenylacetaldehyde (FIG. 2a). In this study, ScARO10*, EipdC and KpPDC from S. cerevisiae, Enterobacter sp. and Komagataella phaffii, respectively were cloned into pET-21a(+) and transformed in E. coli BL21 (DE3), in order to evaluate which of the decarboxylases is the best enzyme for tyrosol production. In that way, the strains ST93, ST135 and ST136 were constructed harbouring ScARO10*, KpPDC and EipdC, respectively. These strains were grown in M9Y with 2% of glucose and induced with 0.1 mM of IPTG for 72 h. Results show that the strain ST93 produces 0.73±0.04 g/L of tyrosol, the strain ST135 could produce 0.31±0.05 g/L of tyrosol and the strain ST136 only produce 0.09±0.01 g/L of tyrosol after 72 h of induction with 0.1 mM of iPTG in M9Y medium (FIG. 2b). Taking this in consideration, the best decarboxylase for tyrosol production was ARO10* since the strain ST93 produced two-fold higher amount of tyrosol than the strain ST135 and produced eightfold higher amount of tyrosol comparing to the strain ST136. Furthermore, once again, higher amounts of tyrosol (ST93) are correlated with lower cell density (OD_{600 nm}) (FIG. 2b)

### Example 4: The Influence of adhP* Overexpression

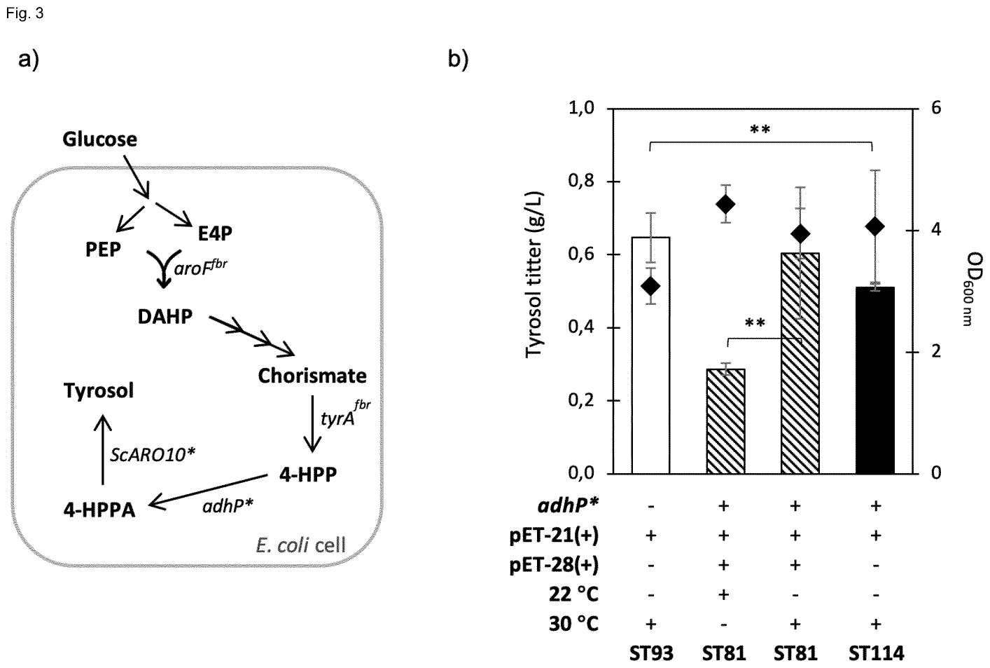

The alcohol dehydrogenase AdhP*, that was kindly provided by Prof. Isabel Rocha group, can reduce 4-hydroxyphenylacetaldehyde into tyrosol and was modified to a better performance for large substrates (FIG. 3a). The adhP* gene was cloned into pET-28a(+) or pET-21a(+) and transformed in E. coli BL21 (DE3), originating the strains ST81 and ST114, respectively to evaluate the influence of overexpression of adhP* into tyrosol production. The strain ST81 could produce 0.60±0.18 g/L of tyrosol and the strain ST114 could produce 0.51±0.01 g/L of tyrosol after 48 h of induction with 0.1 mM of iPTG in M9Y medium (FIG. 3b). Comparing these results with the titer obtained by strain ST93 at the same conditions (0.65±0.07 g/L), that was depicted in FIG. 3b, it was possible to verify that adhP*overexpression did not improve tyrosol production (data not shown), since the titer obtained by strains ST93 and ST81 was not significantly different (p>0.05), while the strain ST114 produced significantly less amount of tyrosol (p<0.01) comparing with strain ST93.

Furthermore, to test the best conditions for AdhP* catalysis, the strain ST81 was induced with 0.1 mM of iPTG in M9Y medium at 22° C. for 48 h. Under these conditions, the strain ST81 could produce 0.29±0.02 g/L of tyrosol (FIG. 3b), which was even lower titer than that obtained when this strain was induced at 30° C. Taking all the results in consideration, the best strain and conditions to produce tyrosol was ST93 after 72 h of induction with 0.1 mM of iPTG in M9Y at 30° C. (0.73±0.04 g/L).

### Example 5: Engineering Aromatic Amino Acid Pathways

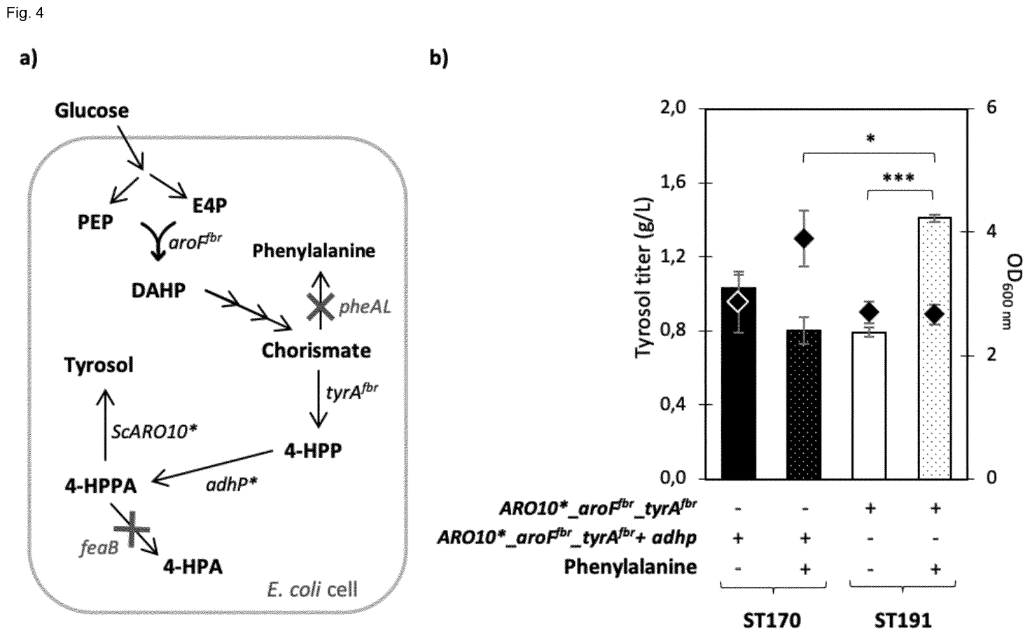

As stated before, endogenous AD H(s) in E. coli are capable of reducing 4-hydroxyphenylacetaldehyde into tyrosol, however this intermediary compound can also be oxidized into 4-hydroxyphenylacetate by an endogenous phenylacetaldehyde dehydrogenase, named FeaB (FIG. 4a). On the other hand, the bifunctional enzyme chorismate mutase/prephenate dehydratase (PheA) is in charge of a very important node in the biosynthesis of phenylalanine and tyrosine, and is responsible for diverting the carbon flux from chorismate toward phenylalanine (FIG. 4a). Consequently, the disruption of these two genes is known to redirect the carbon flux towards tyrosol production. To improve tyrosol production, E. coli BL21 (DE3) strain harbouring knockouts on feaB and pheAL genes (available at SilicoLife's laboratory) served as host to pET-21a(+) with ScARO10*, aroF^{fbr} and tyrA^{fbr} genes, originating the strain ST191. Additionally, the inventors also evaluated the overexpression of adhP* in the feaB and pheAL deletion strain by transforming ScARO10*, aroF^{fbr} and tyrA^{fbr} genes in pET-21a(+) and adhP* gene in pET-28a(+), yielding strain ST170. After growing these two strains the inventors concluded that ST191 produces 0.78±0.02 g/L of tyrosol, while ST170 produces 1.03±0.07 g/L of tyrosol after 96 h of induction with 0.1 mM of IPTG in M9Y medium (FIG. 4b). To notice that growths were prolonged up to 96 h since tyrosol production was still increasing at 72 h of growth. Regarding cell density (OD_{600 nm}) it was possible to verify that in the case of the knockout strains, growth decreased comparing to the respective strains without knockouts (ST93 and 81). Beyond the carbon deviation towards tyrosol, this decrease could be partially explained by a phenylalanine insufficiency caused by the pheAL knockout, which provokes a phenylalanine auxotrophy. Therefore, the inventors suspect that the amount of phenylalanine in M9Y medium which contains 0.025% of yeast extract could not cover the auxotrophy. To test this hypothesis strains ST170 and 191 were induced for 96 h with 0.1 mM of IPTG in M9Y medium supplemented with 20 mg/L of phenylalanine. Under these conditions, the strains ST170 and 191 produces 0.80±0.07 g/L and 1.41±0.02 g/L of tyrosol, respectively (FIG. 4b).

Analysing these results, it is possible to verify that the addition of phenylalanine improves significantly the tyrosol production (p<0.001) on ST191 and decreases for ST170. Furthermore, growth of these strains behaves differently to the addition of phenylalanine, with improved parameters for ST170 and no response in the case of ST191, in comparison with growth with no phenylalanine. In conclusion, the best tyrosol titer from glucose achieved in this work is 1.41±0.02 g/L with strain ST191 corresponding to 10 mM and was attained after 96 h of induction with 0.1 mM of IPTG and addiction of 20 mg/L of phenylalanine in M9Y medium. This result corroborates the titer accomplished by Yang and his collaborators, whose strain produces 1.32 g/L of tyrosol from glucose after 48 h of induction with 0.6 mM of IPTG in M9Y medium by engineering E. coli MG1655 with heterologous expression of ScARO10* and knockout of feaB, pheA, tyrB and tyrR genes (Yang et al., Chinese Journal of Chemical Engineering, 26, 2615-2621). However, in this study the inventors produce 6% more tyrosol than Yang and his team with a strain harbouring ScARO10*, aroF^{fbr} and tyrA^{fbr} genes and with deletions of feaB and pheAL genes. Furthermore, the inventors verify that the heterologous expression of ScARO10* associated with the overexpression of aroF^{fbr} and tyrA^{fbr} in an operon-like system cloned in a pET system improves tyrosol production in approximately 92% in comparison with the first strain constructed (ST53). Additionally, the tyrosol production was enhanced in approximately 50% with the feaB and pheAL gene knockouts in comparison with the strain without these knockouts. On the other hand, AdhP* overexpression did not improve tyrosol production, on the contrary, it decreases 7% in comparison with the strain without this enzyme as discussed above.

Salidroside Production

Salidroside is a phenylethanoid glycoside that was widely distributed in the plant kingdom and has recently attracted increased attention because of its important role in the adaptogenic effect. During the last decade, new metabolic engineering approaches were implemented in E. coli, however more effective strategies are required.

### Example 6: Engineering Salidroside Biosynthesis Pathway in E. coli BL21 (DE3)

The salidroside biosynthesis pathway created in E. coli BL21 (DE3) was achieved by heterologous expression of ScARO10* and AtUGT85A1 genes, and overexpression of aroF^{fbr} and tyrA^{fbr} genes in different plasmids. The critical step of this pathway is the glycosylation of tyrosol into salidroside mediated by uridine diphosphate dependent glycosyltransferase (UGT85A1). This gene was inserted into pET-28a(+) and transformed in E. coli BL21 (DE3) harbouring pET-21a(+)_ScARO10* and in E. coli BL21 (DE3) harbouring pET-21a(+)_ScARO10*_aroF^{fbr}_tyrA^{fbr}, achieving the strains ST95 and ST92, respectively. Both strains were grown aerobically in M9Y medium with glucose and showed a maximum of 0.02±0.01 g/L of salidroside and tyrosol after 48 h of induction with 1 mM of IPTG in M9Y medium for strain ST95 and overexpression of aroF^{fbr} and tyrA^{fbr}, while strain ST92 could produce ten-fold higher titer of salidroside than strain ST95, at the same conditions (0.24±0.05 g/L of salidroside and 0.13±0.03 g/L of tyrosol). This result supports the result obtained by strain ST93 for tyrosol production, which indicated that the overexpression of aroF^{fbr} and tyrA^{fbr} associated with the heterologous expression of ScARO10* enhanced tyrosol production and consequently, salidroside production by UGT85A1.

### Example 7: IPTG Tests and Medium Optimization for Salidroside

With the purpose of verifying if the induction with 0.1 mM of IPTG was also the best concentration for salidroside production, the strain ST92 was induced with 0.1 mM of IPTG for 48 h in M9Y medium. Under these conditions, the strain ST92 produces 0.41±0.07 g/L of salidroside and 0.15±0.04 g/L of tyrosol after 48 h of induction in M9Y medium (Table 11). This result demonstrated that, as well as for tyrosol production, salidroside production was significantly enhanced (p<0.001) by induction with 0.1 mM of IPTG instead of 1 mM of IPTG.

TABLE 11
Tyrosol and salidroside titers (g/L) obtained with strain
ST92 after induction with 0.1 and 1 mM of IPTG in M9Y medium.
Cultures were sampled after 48 h of growth for tyrosol
and salidroside detection. The experiments were independently
conducted three times and experimental data is represented
by the mean ± standard deviation.

| IPTG | Time after | | Tyrosol | Salidroside |
|---|---|---|---|---|
| concentration | induction | | titer | titer |
| (mM) | (h) | OD_{600 nm} | (g/L) | (g/L) |
| 0.1 | 48 | 6.91 ± 0.66 | 0.15 ± 0.04 | 0.41 ± 0.07 |
| 1 | 48 | 3.91 ± 0.77 | 0.13 ± 0.03 | 0.24 ± 0.05 |

However, the strain ST92 metabolism exhibited a bottleneck in salidroside production as tyrosol is accumulated in both concentrations of IPTG that were tested. Different scenarios can explain this accumulation, such as: growth arrest by low pH, consequence of a fermentative metabolism lack of UDP-glucose or other critical nutrient depleted from the medium; or improper enzyme production/folding. Therefore, different M9Y medium compositions were tested in order to see the influence of glucose and pH in salidroside production. For that, the strain ST92 was induced with 0.1 mM of IPTG in M9Y with two-fold amount of salts (2×M9Y) and complemented with 5, 10 or 20 g/L of glucose for 48 h. Under these conditions, the strain ST92 could produce 0.10±0.00 g/L of salidroside and 0.08±0.00 g/L of tyrosol from 5 g/L of glucose, 0.26±0.00 g/L of salidroside and 0.12±0.02 g/L of tyrosol from 10 g/L of glucose, and 0.34±0.01 g/L of salidroside and 0.19±0.00 g/L of tyrosol from 20 g/L of glucose (Table 12). Regarding glucose supply, salidroside production was favoured by addiction of 20 g/L of glucose in 2×M9Y medium, although the best salidroside titer was achieved in M9Y medium complemented with 20 g/L of glucose (0.41±0.07 g/L. This result indicated that buffering the M9Y medium with addiction of two-fold amount of salts did not improve salidroside production.

TABLE 12
Tyrosol and salidroside titers (g/L) obtained with strain
ST92 after induction with 0.1 mM of IPTG in M9Y or 2xM9Y
medium. Cultures were sampled after 48 h of growth for
tyrosol and salidroside detection. The experiments were
independently conducted three times and experimental data
is represented by the mean ± standard deviation.

| | Glucose | | Tyrosol | Salidroside |
|---|---|---|---|---|
| Medium | (g/L) | OD_{600 nm} | titer (g/L) | titer (g/L) |
| 2xM9Y | 5 | 4.90 ± 0.42 | 0.08 ± 0.00 | 0.10 ± 0.00 |
| 2xM9Y | 10 | 6.80 ± 0.28 | 0.12 ± 0.02 | 0.26 ± 0.00 |
| 2xM9Y | 20 | 8.50 ± 0.14 | 0.19 ± 0.00 | 0.34 ± 0.01 |
| M9Y | 20 | 6.91 ± 0.66 | 0.15 ± 0.04 | 0.41 ± 0.07 |

On the other hand, the variation of medium pH was significantly higher in 2×M9Y medium complemented with 20 g/L of glucose (p<0.01) than in 2×M9Y medium supplemented with 5 and 10 g/L of glucose. This pH variation was caused by acetate production, which was higher when 2×M9Y medium was complemented with 20 g/L of glucose. Moreover, the pH variation in M9Y medium and 2×M9Y medium complemented with 20 g/L of glucose was not very significant (p<0.05). Taking all of these in consideration, the best conditions for salidroside production were induction with 0.1 mM of IPTG in M9Y medium complemented with 20 g/L of glucose.

### Example 8: Dynamic Control Over AtUGT85A1 Gene

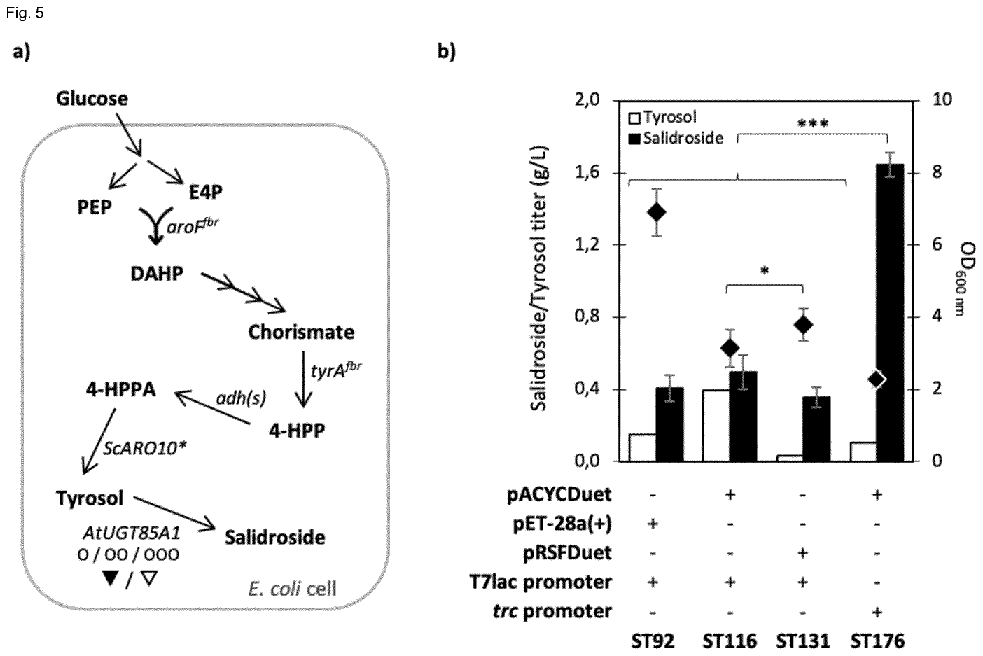

Despite all the attempts for medium optimization, the bottleneck in salidroside production has not been overcome. Thereby, a new strategy was implemented in order to understand if changing the expression level of UGT85A1, by cloning it in different copy number plasmids, would have an effect in salidroside production (FIG. 5a). In that way, AtUGT85A1 was cloned into pACYCDuet (low copy) or pRSFDuet (high copy) plasmid and transformed in E. coli BL21 (DE3) harbouring pET-21a(+)_ScARO10*_aroF^{fbr}_tyrA^{fbr}, obtaining the strains ST116 and ST131, respectively. Growth of these strains shows production values of 0.49±0.10 g/L of salidroside and 0.39±0.06 g/L of tyrosol for ST116 and 0.35±0.06 g/L of salidroside and 0.03±0.00 g/L of tyrosol for ST131. Samples were taken 48 h after induction with 0.1 mM of IPTG and M9Y medium (FIG. 5b). Comparing these results to the one obtained by strain ST92 (0.41±0.07 g/L of salidroside and 0.15±0.04 g/L of tyrosol) it was possible to conclude that although ST92 and 116 were not producing a significantly different amount of salidroside (p>0.05), strain ST116 accumulated more salidroside in absolute values. Also, tyrosol accumulation is higher in ST116 in comparison with ST92. On the other hand, the high-copy plasmid pRSFDuet corresponding to strain ST131 produced the lowest value of salidroside (FIG. 5b).

Additionally, it was possible to verify that increasing the plasmid copy number (pACYCDuet<pET-28a(+)<pRSFDuet) the tyrosol conversion into salidroside was almost totally achieved, however the salidroside titer was not enhanced, indicating that possibly UGT85A1 would be insoluble. Taking this in consideration, the T7 promoter of pACYCDuet_AtUGT85A1 was replaced by trc promoter, originating the strain ST176, in order to optimize tyrosol conversion and salidroside titer. This strain could produce 1.64±0.07 g/L of salidroside and only 0.10±0.06 g/L of tyrosol after 48 h of induction with 0.1 mM of iPTG in M9Y medium (FIG. 5b). Therefore, these results revealed that the conversion of tyrosol into salidroside was almost total and the salidroside titer was improved by heterologous expression of AtUGT85A1 in a low copy number plasmid (pACYCDuet) and under the influence of a lesser strong promoter (trc promoter).

### Example 9: The Influence of feaB and pheAL Gene Knockouts

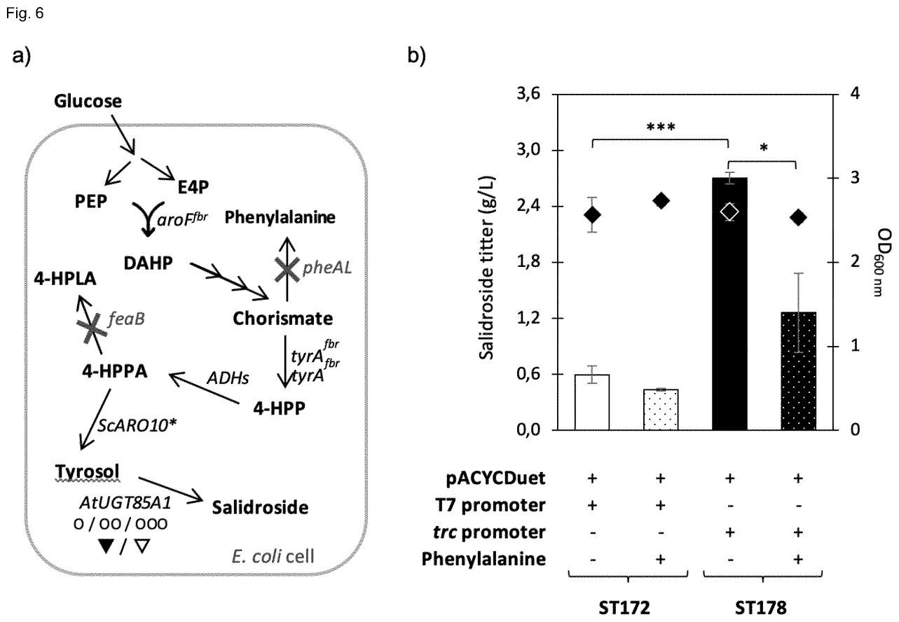

To improve metabolic flow towards salidroside, the inventors set to clone the best two gene organizations and attempt to improve its production, the best gene organizations were cloned into E. coli BL21 (DE3) harbouring feaB and pheAL gene knockouts (FIG. 6a), originating the strain ST172 with pET-21a(+)_ScARO10*_aroF^{fbr}_tyrA^{fbr} and pACYCDuet_AtUGT85A1 and the strain ST178 with pET-21a(+)_ScARO10*_aroF^{fbr}_tyrA^{fbr} and pACYCDuet_trc-pm_AtUGT85A1. The strain ST172 could produce 0.59±0.09 g/L of salidroside and 0.80±0.08 g/L of tyrosol and the strain ST178 could produce 2.70±0.06 g/L of salidroside and 0.09±0.02 g/L of tyrosol after 96 h of induction with 0.1 mM of IPTG in M9Y medium (FIG. 6b).

Once again, in ST178, tyrosol at a major extent is converted into salidroside and as observed before, ST172 accumulated salidroside in conjunction with significant amounts of tyrosol. In conclusion, cloning AtUGT85A1 in a low copy plasmid and under the influence of a weaker promoter balanced the production of the protein and improved significantly the salidroside titers. Furthermore, it was also possible to verify that the knockouts improved salidroside production in both strains, comparing to the respective strains without knockouts.

Besides that, the influence of phenylalanine supplementation was also evaluated on salidroside production. For that, the strains ST172 and ST178 were induced for 96 h with 0.1 mM of IPTG in M9Y medium supplemented with 20 mg/L of phenylalanine. Under these conditions, the strain ST172 could produce 0.43±0.01 g/L of salidroside and 0.90±0.03 g/L of tyrosol and the strain ST178 could produce 1.25±0.42 g/L of salidroside and 0.40±0.12 g/L of tyrosol (FIG. 6b). These results demonstrated that the addition of phenylalanine decrease the salidroside production, contrarily to what happens with tyrosol (data not shown). Accordingly, the best salidroside titer from glucose accomplished in this study was produced by strain ST178 (3.11±0.19 g/L of salidroside) after 121 h of induction with 0.1 mM of IPTG in M9Y medium complemented with 20 g/L of glucose. This result corresponds to approximately ten-fold higher amount of salidroside than that obtained by Chung and is team, which only produced 0.28 g/L of salidroside from glucose after 48 h of induction with 1 mM of IPTG in M9Y medium at 25° C. by engineering E. coli BL21 (DE3) with heterologous expression of PcAAS and AtUGT85A1 and knockout of tyrR, pheA and feaB genes (Chung, et al, Escherichia coli. Scientific Reports, 7, 1-8, (2017)).

Hydroxytyrosol Production

Hydroxytyrosol is one of the most abundant phenolic alcohols in olives and have some exceptional features that makes it ideal for implementation in the nutraceutical, agrochemical, cosmeceutical and food industry. However, besides all the work already done, a cost-effectively approach was not found yet.

### Example 10: Overexpressinq hpaBC* in E. coli BL21 (DE3)

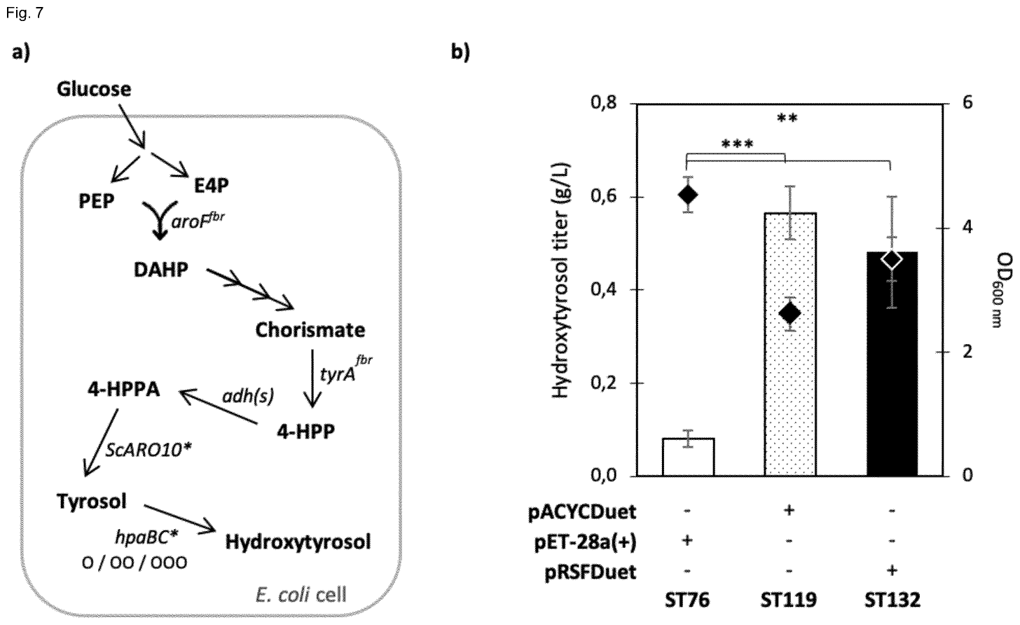

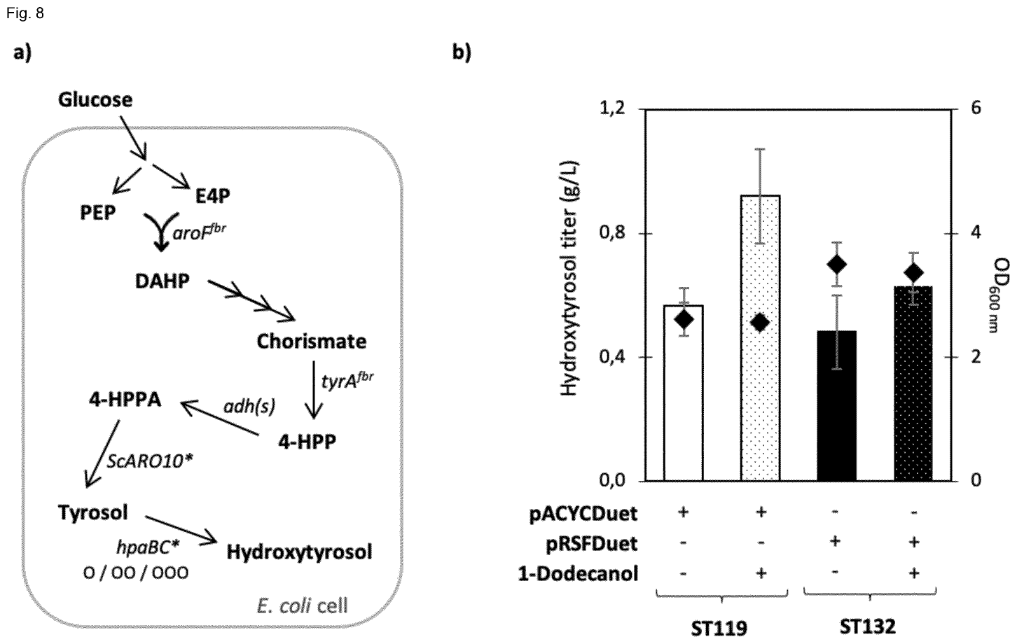

The fundamental step in hydroxytyrosol biosynthesis is the conversion of tyrosol into hydroxytyrosol. To mediate this step there are several possible candidate enzymes described in literature. Espin and his team used a mushroom tyrosinase, however this enzyme is unstable and its activity is inhibited by phenols and ascorbic acid. Another study conducted by Liebgott and his co-workers demonstrated that 4-hydroxyphenylacetic acid 3-hydroxylase from different bacteria was responsible of converting tyrosol into hydroxytyrosol. Furthermore, other native hydrolases of some aromatic compound degrading microorganisms, such as Serratia marcescens, Pseudomonas aeruginosa, Pseudomonas putida F6 and Halomonas sp. strain HTB24 were identified to convert tyrosol into hydroxytyrosol. More recently, 4-hydroxyphenylacetate 3-monooxygenase (HpaBC*) was engineered from E. coli in order to improve its activity and specificity for tyrosol. With this engineered enzyme they achieved a high activity for tyrosol and founded that its docking energy for tyrosol was much lower than that for wild-type HpaBC. So, in this study, HpaBC* was selected from all enzymes since it is an endogenous enzyme of E. coli and was engineered for a better performance from tyrosol as a substrate. That way, the hydroxytyrosol biosynthesis pathway was implemented in E. coli BL21 (DE3) by heterologous expression of ScARO10* gene and overexpression of aroF^{fbr}, tyrA^{fbr} and hpaBC* genes (FIG. 7a). In this line of thought, three strains were constructed to evaluate the influence of plasmid copy number in hpaBC*overexpression and, consequently hydroxytyrosol production. All the three strains harbour pET-21a(+)_ScARO10*_aroF^{fbr}_tyrA^{fbr}, however the hpaBC* was cloned in pET-28a(+) for strain ST76, in pACYCDuet for strain ST119 and in pRSFDuet for strain ST132. Then all the strains were induced for 48 h with 0.1 mM of IPTG in M9Y medium supplemented with 1 g/L of ascorbic acid to avoid hydroxytyrosol oxidation. Under these conditions, the strain ST76 produces 0.08±0.02 g/L of hydroxytyrosol, the strain ST119 produces 0.57±0.06 g/L of hydroxytyrosol, and the strain ST132 produces 0.48±0.12 g/L of hydroxytyrosol (FIG. 7b). For all strains, residual amounts of tyrosol were accumulated (<80 mg/L). These results were not congruous, since ST119 and 132, with low and high copy plasmid, respectively, did not produced a significantly different amount of hydroxytyrosol (p>0.05). However, it is important to notice that hydroxytyrosol production in ST132 is more irregular than strains ST119 and 76, which is an indication of plasmid instability. On the other hand, strain ST76 that has a medium copy plasmid is the strain producing less hydroxytyrosol than the other two strains. Moreover, the strain that demonstrated a lower cell density (OD_{600 nm}) was strain ST119, which was the strain that produce more hydroxytyrosol, as observed for tyrosol and salidroside. Also, toxicity towards hydroxytyrosol has not been reported to concentrations below 1 g/L of hydroxytyrosol. On the other hand, during growth of this strains the inventors noticed that culture medium changed to a darker colour indicating the oxidation of media components, which included hydroxytyrosol.

### Example 11: The Influence of a Biphasic Growth

As stated before, hydroxytyrosol is an antioxidant easily oxidized during its production, making this compound more unstable than tyrosol or salidroside. Besides that, it was reported that hydroxytyrosol shows an inhibitory effect on cell growth above 1 g/L. Taking this in consideration, the inventors designed a biphasic growth with 1-dodecanol that could sequester hydroxytyrosol, avoid its oxidation and cell toxicity. To do so, the inventors added 25% (v/v) of 1-dodecanol to the culture media when growth was no longer observed, which occurs 16 h after protein induction. Maximal production was detected at 48 h of induction with 0.1 mM of IPTG in M9Y medium supplemented with 1 g/L of ascorbic acid and addition of 12.5 ml of 1-dodecanol (FIG. 8b). Results show that strains ST119 and 132 were able to produce 0.92±0.15 g/L and 0.63±0.06 g/L, respectively, and trace amounts of tyrosol. Comparing hydroxytyrosol titers obtained by strains ST119 and 132 with or without addition of 1-dodecanol, it is possible to verify that in the biphasic system the strains ST119 and 132 increased their production in more than 30% and 20%, respectively. However, the cell density was not improved, showing that growth arrest is not associated with hydroxytyrosol accumulation. These results confirmed that the biphasic system stabilizes hydroxytyrosol production and revealed that hydroxytyrosol titer improves when hpaBC* is cloned in a low copy plasmid (ST119) in comparison with high copy plasmid in strain ST132.

### Example: 12 IPTG Optimization

Such as for tyrosol and salidroside, different IPTG concentrations were tested to evaluate the best induction condition for hydroxytyrosol production. In this case, the strain ST119 were induced with 0.1 mM and 0.2 mM of IPTG for 48 h in M9Y medium supplemented with 1 g/L of ascorbic acid and addition of 12.5 ml of 1-dodecanol. The strain ST119 produced 0.56±0.09 g/L of hydroxytyrosol and trace amounts of tyrosol after induction with 0.2 mM of IPTG, which was significantly less than the hydroxytyrosol titer obtained when strain ST119 was induced with 0.1 mM of IPTG (0.92±0.15 g/L of hydroxytyrosol) (Table 13). Furthermore, the cell density (OD_{600 nm}) was not affected when the cells were induced with 0.1 or 0.2 mM of IPTG despite the different accumulated amounts of hydroxytyrosol. With this result was possible to realise that the best conditions for hydroxytyrosol production were induction for 48 h with 0.1 mM of IPTG in M9Y medium supplemented with 1 g/L of ascorbic acid and addition of 12.5 ml of 1-dodecanol. To evaluate the solubility of ARO10*, AroF^{fbr}, TyrA^{fbr} and HpaBC* proteins whose genes were overexpressed in the pET system, a SDS-PAGE gel was performed which shows that overproduced proteins are mainly soluble.

TABLE 13
Tyrosol and hydroxytyrosol titers (g/L) achieved with strain
ST119 after induction with 0.1 and 0.2 mM of IPTG in M9Y
medium supplemented with 1 g/L of ascorbic acid and associated
with addition of 25% (v/v) of 1-dodecanol. Cultures were
sampled after 48 h of growth for tyrosol and hydroxytyrosol
detection. The experiments were independently conducted
three times and experimental data is represented by the
mean ± standard deviation.

| IPTG | Time after | | Tyrosol | Hydroxytyrosol |
|---|---|---|---|---|
| concentration | induction | | titer | titer |
| (mM) | (h) | OD_{600 nm} | (g/L) | (g/L) |
| 0.1 | 48 | 2.57 ± 0.12 | 0.06 ± 0.01 | 0.92 ± 0.15 |
| 0.2 | 48 | 2.57 ± 0.57 | 0.01 ± 0.00 | 0.56 ± 0.09 |

In conclusion, the best condition for hydroxytyrosol production was 6 mM and was obtained with strain ST119, 48 h after induction with 0.1 mM of IPTG in M9Y medium supplemented with 1 g/L of ascorbic acid and 20 g/L of glucose and addition of 12.5 ml of 1-dodecanol. Under these conditions, it was possible to accumulate 0.92±0.15 g/L of hydroxytyrosol, which corresponds to an increase of approximately 40% in comparison to the production without 1-dodecanol and up to the inventors' knowledge is the best hydroxytyrosol titer reported. However, the tyrosol conversion into hydroxytyrosol was not very efficient since only 60% of tyrosol was converted into hydroxytyrosol, comparing with tyrosol strain ST191. Hydroxytyrosol production in E. coli has been reported before (0.65 g/L of hydroxytyrosol) from glucose, by engineering E. coli BW25113 with heterologous expression of ScARO10 gene, overexpression of ADH6, tyrA, ppsA, tktA and aroG genes, and knocking out feaB gene. They achieved this production by inducing cells with 0.5 mM of IPTG in M9Y medium at 37° C. Comparing this result to the one obtained in this study, Li and his team produced approximately 30% less hydroxytyrosol, which could be explained by the use of 0.5 mM of IPTG instead of 0.1 mM of IPTG, overexpressing more genes than us and knocking out only feaB gene.

### Example 13: Hydroxytyrosol Production in E. coli with HT1 Pathway

TABLE 14
shows strain, media composition and respective titer

| Strain | Genome | Genotype | Significant differences |
|---|---|---|---|
| Strain | Media | | HT Titer (g/L) |
| HT1 | Regular media | | 0.12 |
| (ST76) | Regular media + 1 g/L ascorbic acid | | 0.08 |
| HT2 | Regular media + 1 g/L ascorbic acid | | 0.57 |
| (ST119) | Regular media + 1 g/L ascorbic acid + 25% dodecanol (v/v) | | 1.00 |
| | Regular media + 1 g/L ascorbic acid + O₂ limitation | | 0.30 |
| HT3 | Regular media + 1 g/L ascorbic acid | | 0.45 |
| (ST132) | Regular media + 1 g/L ascorbic acid + 25% dodecanol (v/v) | | 0.63 |
| HT4 | Regular media + 1 g/L ascorbic acid | | 0.44 |
| (ST173) | Regular media + 1 g/L ascorbic acid + 25% dodecanol (v/v) | | 0.42 |
| | Regular media + 1 g/L ascorbic acid + O₂ limitation | | 0.20 |
| HT1 | BL21(DE3) | | pET21a:ScARO10:tyrA^{fbr}:aroF^{fbr} + |
| (ST76) | | | pet28a:hpaBC* |

TABLE 15
Strain description:

| Strain | Genome | Genotype | Significant differences |
|---|---|---|---|
| HT1 | BL21(DE3) | pET21a:ScARO10:tyrA^{fbr}:aroF^{fbr} + | Pet28a - medium copy |
| (ST76) | | pet28a:hpaBC* | plasmid from hpaBC that |
| | | | converts tyrosol in hydrotyrosol |
| HT2 | BL21(DE3) | pET21a:ScARO10:tyrA^{fbr}:aroF^{fbr} + | pacycduet - low copy plasmid |
| (ST119) | | pACYCduet:hpaBC* | from hpaBC that converts |
| | | | tyrosol in hydrotyrosol |
| HT3 | BL21(DE3) | pET21a:ScARO10:tyrA^{fbr}:aroF^{fbr} + | Pet28a - high copy plasmid |
| (ST132) | | pRSFduet:hpaBC* | from hpaBC that converts |
| | | | tyrosol in hydrotyrosol |
| HT4 | BL21(DE3)ΔpheaLΔfeaB | pET21a:ScARO10:tyrA^{fbr}:aroF^{fbr} + | Equal to HT2 with knockout of |
| (ST173) | | pACYCduet:hpaBC* | competing pathways |

Cells were grown in LB medium for 2 h, washed and resuspended in M9Y+2% of glucose+0.1 mM of IPTG (regular media)) at 30 C and incubated for 72 h. The low copy number for hpaBC favours the accumulation of hydroxytyrosol. The addition of dodecanol increased the hydroxytyrosol production in approximately 40%. The biphasic system stabilized hydroxytyrosol production. The pheaL and feaB gene knockouts and the 02 limitation decreased the hydroxytyrosol accumulation.

1. A method for production of tyrosol, wherein a transgenic bacterial cell that heterologously expresses:
a. phenylpyruvate decarboxylase (ARO10)
and that overexpresses each of:
b. phospho-2-dehydro-3-deoxyheptonate aldolase (aroF)
c. prephenate dehydrogenase (tyrA)
and wherein each of the following genes is not expressed:
i. pheAL (bifunctional chorismate mutase/prephenate dehydratase)
ii. feaB (phenylacetaldehyde dehydrogenase)
is grown in a medium comprising
a metabolic precursor of phosphoenolpyruvate (PEP) and erythrose 4-phosphate (E4P), particularly wherein the metabolic precursor is glucose, and
optionally, phenylalanine as a supplement;
and tyrosol is extracted from said medium.

2. The method according to claim 1, wherein the transgenic bacterial cell is of the genus Escherichia, particularly wherein the transgenic bacterial cell is of the species E. coli, more particularly wherein the transgenic bacterial cell is of the strain E. coli BL21.

3. The method according to claim 1, wherein the gene encoding the phenylpyruvate decarboxylase originates from yeast, particularly from S. cerevisiae.

4. A method for production of salidroside, wherein
a transgenic bacterial cell according to claim 1 additionally heterologously expresses uridine diphosphate dependent glycosyltransferase (UGT85A1), and
the cell is grown in a medium comprising
a metabolic precursor of phosphoenolpyruvate (PEP) and erythrose 4-phosphate (E4P), particularly glucose, and
optionally, phenylalanine as a supplement;
and salidroside is extracted from said medium.

5. The method according to claim 4, wherein the gene encoding uridine diphosphate dependent glycosyltransferase originates from a plant, particularly from Arabidopsis, more particularly from A. thaliana.

6. The method according to claim 1, wherein the transgenic bacterial cell does not overexpress any of the following proteins:
alcohol dehydrogenase,
DNA-binding transcriptional regulatory protein (tyrR),
and
tyrosine aminotransferase.

7. The method according to claim 1, wherein the only heterologously expressed genes of the transgenic bacterial cell are
i) wherein the method is directed at the production of tyrosol, the only heterologously expressed gene in the cell is phenylpyruvate decarboxylase;
ii) wherein the method is directed at the production of salidroside, the only heterologously expressed genes in the cell are phenylpyruvate decarboxylase and uridine diphosphate dependent glycosyltransferase.

8. The method according to claim 1, wherein the overexpressed genes and the transgenes are introduced into the transgenic bacterial cell via one or several plasmid vector(s), particularly wherein
phenylpyruvate decarboxylase, phospho-2-dehydro-3-deoxyheptonate aldolase and prephenate dehydrogenase are encoded by a medium-copy plasmid vector, and/or
uridine diphosphate dependent glycosyltransferase is encoded by a low-copy plasmid vector.

9. The method according to claim 1, wherein said transgenic bacterial cell comprises one or more plasmids encoding said heterologously expressed or overexpressed enzymes under control of a promoter sequence operable in said cell, particularly a T7 promoter (SEQ ID NO. 31), a lac promoter (SEQ ID NO. 32), a tac promoter (SEQ ID NO. 33) or a trc promoter (SEQ ID NO. 34), more particularly wherein
the gene encoding uridine diphosphate dependent glycosyltransferase is under control of a trc promoter, and/or
the gene encoding phenylpyruvate decarboxylase is under control of a T7 promoter, and/or
the gene encoding phospho-2-dehydro-3-deoxyheptonate aldolase is under control of a T7 promoter, and/or
the gene encoding prephenate dehydrogenase is under control of a T7 promoter.

10. The method according to claim 9, wherein the expression of said heterologous and/or overexpressed genes is induced by adding isopropyl-β-d-thiogalactopyranoside (IPTG), particularly at a concentration of ˜0.1 mM IPTG for 96 h.

11. The method according to claim 1, wherein said medium comprises 10 to 50 g/L of glucose, particularly 15 to 30 g/L of glucose.

12. The method according to claim 1, wherein the transgenes are codon-optimized for expression in said transgenic bacterial cell.

13. The method according to claim 1, wherein the medium comprises:
5-10 g/L Na₂HPO₄·2H₂O,
2-4 g/L KH₂PO₄,
0.25-1 g/L NaCl,
0.5-1.5 g/L NH₄Cl,
1-3% (w/v) glucose,
0.01-0.05% (w/v) yeast extract,
3-7 mM MgSO₄,
0.005-0.02 g/L CaCl₂
and
antibiotics,
particularly wherein the antibiotics are 50-200 μg/mL ampicillin, 10-50 μg/mL kanamycin and 25-45 μg/mL chloramphenicol.

14. The method according to claim 1, wherein
a. the phenylpyruvate decarboxylase has at least 60%, 65%, 70%, 75%, 80%, particularly 85%, more particularly 90%, even more particularly 95% or yet even more particularly >95% sequence identity with SEQ ID NO 1, and wherein the phenylpyruvate decarboxylase has a catalytic activity of at least 75% of the activity of SEQ ID NO 1 and/or
b. the phospho-2-dehydro-3-deoxyheptonate aldolase has at least 60%, 65%, 70%, 75%, 80%, particularly 85%, more particularly 90%, even more particularly 95% or yet even more particularly >95% sequence identity with SEQ ID NO 2 and wherein the phospho-2-dehydro-3-deoxyheptonate aldolase has a catalytic activity of at least 75% of the activity of SEQ ID NO 2 and/or
c. the prephenate dehydrogenase has at least 60%, 65%, 70%, 75%, 80%, particularly 85%, more particularly 90%, even more particularly 95% or yet even more particularly >95% sequence identity with SEQ ID NO 3 and wherein the prephenate dehydrogenase has a catalytic activity of at least 75% of the activity of SEQ ID NO 3 and/or
d. the uridine diphosphate dependent glycosyltransferase has at least 60%, 65%, 70%, 75%, particularly 85%, more particularly 90%, even more particularly 95% or yet even more particularly >95% sequence identity with SEQ ID NO 4 and wherein the uridine diphosphate dependent glycosyltransferase has a catalytic activity of at least 75% of the activity of SEQ ID NO 4.

15. A transgenic cell according to claim 1.


SEQUENCE LISTING (41 sequences)

SEQ ID NO: 1 - protein, 635 aa, Saccharomyces cerevisiae
MAPVTIEKFVNQEERHLVSNRSATIPFGEYIFKRLLSIDTKSVFGVPGDFNLSLLEYLYSPSVESAGLRWVGTCNELNAAYAADGYSRYSNKIGCLITTYGVGELSALNGIAGSFAENVKVLHIVGVAKSIDSRSSNFSDRNLHHLVPQLHDSNFKGPNHKVYHDMVKDRVACSVAYLEDIETACDQVDNVIRDIYKYSKPGYIFVPADFADMSVTCDNLVNVPRISQQDCIVYPSENQLSDIINKITSWIYSSKTPAILGDVLTDRYGVSNFLNKLICKTGIWNFSTVMGKSVIDESNPTYMGQYNGKEGLKQVYEHFELCDLVLHFGVDINEINNGHYTFTYKPNAKIIQFHPNYIRLVDTRQGNEQMFKGINFAPILKELYKRIDVSKLSLQYDSNVTQYTNETMRLEDPTNGQSSIITQVHLQKTMPKFLNPGDVVVCETGSFQFSVRDFAFPSQLKYISQGFFLSIGMALPAALGVGIAMQDHSNAHINGGNVKEDYKPRLILFEGDGAAQMTIQELSTILKCNIPLEVIIWNNNGYTIERAIMGPTRSYNDVMSWKWTKLFEAFGDFDGKYTNSTLIQCPSKLALKLEELKNSNKRSGIELLEVKLGELDFPEQLKCMVEAAALKRNKK

SEQ ID NO: 2 - protein, 356 aa, Escherichia coli
MQKDALNNVHITDEQVLMTPEQLKAAFPLSLQQEAQIADSRKSISDIIAGRDPRLLVVCGPCSIHDPETALEYARRFKALAAEVSDSLYLVMRVYFEKPRTTVGWKGLINDPHMDGSFDVEAGLQIARKLLLELVNMGLPLATEALNPNSPQYLGDLFSWSAIGARTTESQTHREMASGLSMPVGFKNGTDGSLATAINAMRAAAQPHRFVGINQAGQVALLQTQGNPDGHVILRGGKAPNYSPADVAQCEKEMEQAGLRPSLMVDCSHGNSNKDYRRQPAVAESVVAQIKDGNRSIIGLMIESNIHEGNQSSEQPRSEMKYGVSVTDACISWEMTDALLREIHQDLNGQLTARVA

SEQ ID NO: 3 - protein, 373 aa, Escherichia coli
MVAELTALRDQIDEVDKALLNLLAKRLELVAEVGEVKSRFGLPIYVPEREASILASRRAEAEALGVPPDLIEDVLRRVMRESYSSENDKGFKTLCPSLRPVVIVGGGGQMGRLFEKMLTLSGYQVRILEQHDWDRAADIVADAGMVIVSVPIHVTEQVIGKLPPLPKDCILVDLASVKNGPLQAMLVAHDGPVLGLHPMFGPDSGSLAKQVVVWCDGRKPEAYQWFLEQIQVWGARLHRISAVEHDQNMAFIQALRHFATFAYGLHLAEENVQLEQLLALSSPIYRLELAMVGRLFAQDPQLYADIIMSSERNLALIKRYYKRFGEAIELLEQGDKQAFIDSFRKVEHWFGDYVQRFQSESRVLLRQANDNRQ

SEQ ID NO: 4 - protein, 489 aa, Arabidopsis thaliana
MGSQIIHNSQKPHVVCVPYPAQGHINPMMRVAKLLHARGFYVTFVNTVYNHNRFLRSRGSNALDGLPSFRFESIADGLPETDMDATQDITALCESTMKNCLAPFRELLQRINAGDNVPPVSCIVSDGCMSFTLDVAEELGVPEVLFWTTSGCAFLAYLHFYLFIEKGLCPLKDESYLTKEYLEDTVIDFIPTMKNVKLKDIPSFIRTTNPDDVMISFALRETERAKRASAIILNTFDDLEHDVVHAMQSILPPVYSVGPLHLLANREIEEGSEIGMMSSNLWKEEMECLDWLDTKTQNSVIYINFGSITVLSVKQLVEFAWGLAGSGKEFLWVIRPDLVAGEEAMVPPDFLMETKDRSMLASWCPQEKVLSHPAIGGFLTHCGWNSILESLSCGVPMVCWPFFADQQMNCKFCCDEWDVGIEIGGDVKREEVEAVVRELMDGEKGKKMREKAVEWQRLAEKATEHKLGSSVMNFETVVSKFLLGQKSQD

SEQ ID NO: 5 - DNA, 1071 bp, Escherichia coli
atgcaaaaagacgcgctgaataacgtacatattaccgacgaacaggttttaatgactccggaacaactgaaggccgcttttccattgagcctgcaacaagaagcccagattgctgactcgcgtaaaagcatttcagatattatcgccgggcgcgatcctcgtctgctggtagtatgtggtccttgttccattcatgatccggaaactgctctggaatatgctcgtcgatttaaagcccttgccgcagaggtcagcgatagcctctatctggtaatgcgcgtctattttgaaaaaccccgtaccactgtcggctggaaagggttaattaacgatccccatatggatggctcttttgatgtagaagccgggctgcagatcgcgcgtaaattgctgcttgagctggtgaatatgggactgccactggcgacggaagcgttagatccgaatagcccgcaatacctgggcgatctgtttagctggtcagcaattggtgctcgtacaacggaatcgcaaactcaccgtgaaatggcctccgggctttccatgccggttggttttaaaaacggcaccgacggcagtctggcaacagcaattaacgctatgcgcgccgccgcccagccgcaccgttttgttggcattaaccaggcagggcaggttgcgttgctacaaactcaggggaatccggacggccatgtgatcctgcgcggtggtaaagcgccgaactatagccctgcggatgttgcgcaatgtgaaaaagagatggaacaggcgggactgcgcccgtctctgatggtagattgcagccacggtaattccaataaagattatcgccgtcagcctgcggtggcagaatccgtggttgctcaaatcaaagatggcaatcgctcaattattggtctgatgatcgaaagtaatatccacgagggcaatcagtcttccgagcaaccgcgcagtgaaatgaaatacggtgtatccgtaaccgatgcctgcattagctgggaaatgaccgatgccttgctgcgtgaaattcatcaggatctgaacgggcagctgacggctcgcgtggcttaa

SEQ ID NO: 6 - DNA, 1071 bp, Artificial Sequence
  aroFfbr (D147N)
atgcaaaaagacgcgctgaataacgtacatattaccgacgaacaggttttaatgactccggaacaactgaaggccgcttttccattgagcctgcaacaagaagcccagattgctgactcgcgtaaaagcatttcagatattatcgccgggcgcgatcctcgtctgctggtagtatgtggtccttgttccattcatgatccggaaactgctctggaatatgctcgtcgatttaaagcccttgccgcagaggtcagcgatagcctctatctggtaatgcgcgtctattttgaaaaaccccgtaccactgtcggctggaaagggttaattaacgatccccatatggatggctcttttgatgtagaagccgggctgcagatcgcgcgtaaattgctgcttgagctggtgaatatgggactgccactggcgacggaagcgttaaatccgaatagcccgcaatacctgggcgatctgtttagctggtcagcaattggtgctcgtacaacggaatcgcaaactcaccgtgaaatggcctccgggctttccatgccggttggttttaaaaacggcaccgacggcagtctggcaacagcaattaacgctatgcgcgccgccgcccagccgcaccgttttgttggcattaaccaggcagggcaggttgcgttgctacaaactcaggggaatccggacggccatgtgatcctgcgcggtggtaaagcgccgaactatagccctgcggatgttgcgcaatgtgaaaaagagatggaacaggcgggactgcgcccgtctctgatggtagattgcagccacggtaattccaataaagattatcgccgtcagcctgcggtggcagaatccgtggttgctcaaatcaaagatggcaatcgctcaattattggtctgatgatcgaaagtaatatccacgagggcaatcagtcttccgagcaaccgcgcagtgaaatgaaatacggtgtatccgtaaccgatgcctgcattagctgggaaatgaccgatgccttgctgcgtgaaattcatcaggatctgaacgggcagctgacggctcgcgtggcttaa

SEQ ID NO: 7 - DNA, 1470 bp, Arabidopsis thaliana
atgggatctcagatcattcataactcacaaaaaccacatgtagtttgtgttccatatccggctcaaggccacatcaaccctatgatgagagtggctaaactcctccacgccagaggcttctacgtcaccttcgtcaacaccgtctacaaccacaatcgtttccttcgttctcgtgggtccaatgccctagatggacttccttcgttccgatttgagtccattgctgacggtctaccagagacagacatggatgccacgcaggacatcacagctctttgcgagtccaccatgaagaactgtctcgctccgttcagagagcttctccagcggatcaacgctggagataatgttcctccggtaagctgtattgtatctgacggttgtatgagctttactcttgatgttgcggaggagcttggagtcccggaggttcttttttggacaaccagtggctgtgcgttcctggcttatctacacttttatctcttcatcgagaagggcttatgtccgctaaaagatgagagttacttgacgaaggagtacttagaagacacggttatagattttataccaaccatgaagaatgtgaaactaaaggatattcctagcttcatacgtaccactaatcctgatgatgttatgattagtttcgccctccgcgagaccgagcgagccaaacgtgcttctgctatcattctaaacacatttgatgaccttgagcatgatgttgttcatgctatgcaatctatcttacctccggtttattcagttggaccgcttcatctcttagcaaaccgggagattgaagaaggtagtgagattggaatgatgagttcgaatttatggaaagaggagatggagtgtttggattggcttgatactaagactcaaaatagtgtcatttatatcaactttgggagcataacggttttgagtgtgaagcagcttgtggagtttgcttggggtttggcgggaagtgggaaagagtttttatgggtgatccggccagatttagtagcgggagaggaggctatggttccgccggactttttaatggagactaaagaccgcagtatgctagcgagttggtgtcctcaagagaaagtactttctcatcctgctattggagggtttttgacgcattgcgggtggaactcgatattggaaagtctttcgtgtggagttccgatggtgtgttggccattttttgctgaccagcaaatgaattgtaagttttgttgtgacgagtgggatgttgggattgagataggtggagatgtgaagagagaggaagttgaggcggtggttagagagctcatggatggagagaagggaaagaaaatgagagaaaaggcggtagagtggcagcgcttagccgagaaagcgacggaacataaacttggttcttccgttatgaattttgagacggttgttagcaagtttcttttgggacaaaaatcacaggattaa

SEQ ID NO: 8 - DNA, 1470 bp, Artificial Sequence
  optimized AtUGT85A1
atgggatcacagatcatacacaactcgcagaaaccccatgtggtctgtgtaccgtaccccgctcagggccatattaatccaatgatgcgtgtcgctaaattacttcatgccagagggttttatgtaacattcgtcaatacagtgtataatcacaatagatttcttagaagccgcgggtcgaatgcgttagacggcctgccctccttccggtttgagtcaatagccgacgggttgcctgaaacggatatggacgccacacaggacataacggctctgtgtgagtcgactatgaagaattgtctggctcccttccgcgagttgctgcaacggataaatgctggggataacgtacctcctgttagctgtatagtatccgatgggtgcatgtcctttacccttgatgtagcagaggaacttggagtgccggaagttttgttctggaccactagcgggtgtgcctttttagcatacctgcacttttatttatttatagaaaaggggttgtgtcccttaaaagacgagtcctatttgacgaaggaataccttgaggacacggttatagatttcataccgactatgaaaaacgttaagctgaaggatatacctagcttcatacgcacaactaatccggatgatgttatgatctcttttgccctgcgtgagacagagcgcgctaagcgggcgtctgcgattatattgaacacatttgacgatctggaacacgatgttgtccacgcaatgcagtccattcttcctccggtatattcagtgggacccttgcaccttttagcgaaccgggaaatcgaagaaggatctgaaataggtatgatgtcttccaacttatggaaggaagaaatggagtgtcttgactggttggatacaaagacacaaaattccgtaatatacataaacttcggcagcatcacggtgttgagcgtaaaacagctggtcgaattcgcttggggtttggcaggttccggtaaggagttcttgtgggtcataagaccagacttagtcgcgggggaagaagcaatggtaccccccgacttccttatggagacgaaagaccgttccatgttggcctcttggtgccctcaagagaaagtcttgtcacatcccgctattggagggttcctgacacactgtggttggaattcaattcttgagagcttatcgtgtggggtgccaatggtgtgctggccgttctttgcagatcagcaaatgaactgtaagttttgctgcgacgaatgggatgtaggtatagagatcggcggcgacgttaagcgcgaggaggtcgaggcagttgtaagagagctgatggacggtgagaaaggcaaaaaaatgagagaaaaagcggtcgagtggcagcggttggctgagaaagctacggaacataaacttggcagtagcgttatgaactttgaaactgttgtatcgaaatttttgctggggcagaaaagccaggactaa

SEQ ID NO: 9 - DNA, 1908 bp, Saccharomyces cerevisiae
atggcacctgttacaattgaaaagttcgtaaatcaagaagaacgacaccttgtttccaaccgatcagcaacaattccgtttggtgaatacatatttaaaagattgttgtccatcgatacgaaatcagttttcggtgttcctggtgacttcaacttatctctattagaatatctctattcacctagtgttgaatcagctggcctaagatgggtcggcacgtgtaatgaactgaacgccgcttatgcggccgacggatattcccgttactctaataagattggctgtttaataaccacgtatggcgttggtgaattaagcgccttgaacggtatagccggttcgttcgctgaaaatgtcaaagttttgcacattgttggtgtggccaagtccatagattcgcgttcaagtaactttagtgatcggaacctacatcatttggtcccacagctacatgattcaaattttaaagggccaaatcataaagtatatcatgatatggtaaaagatagagtcgcttgctcggtagcctacttggaggatattgaaactgcatgtgaccaagtcgataatgttatccgcgatatttacaagtattctaaacctggttatatttttgttcctgcagattttgcggatatgtctgttacatgtgataatttggttaatgttccacgtatatctcaacaagattgtatagtatacccttctgaaaaccaattgtctgacataatcaacaagattactagttggatatattccagtaaaacacctgcgatccttggagacgtactgactgataggtatggtgtgagtaactttttgaacaagcttatctgcaaaactgggatttggaatttttccactgttatgggaaaatctgtaattgatgagtcaaacccaacttatatgggtcaatataatggtaaagaaggtttaaaacaagtctatgaacattttgaactgtgcgacttggtcttgcattttggagtcgacatcaatgaaattaataatgggcattatacttttacttataaaccaaatgctaaaatcattcaatttcatccgaattatattcgccttgtggacactaggcagggcaatgagcaaatgttcaaaggaatcaattttgcccctattttaaaagaactatacaagcgcattgacgtttctaaactttctttgcaatatgattcaaatgtaactcaatatacgaacgaaacaatgcggttagaagatcctaccaatggacaatcaagcattattacacaagttcacttacaaaagacgatgcctaaatttttgaaccctggtgatgttgtcgtttgtgaaacaggctcttttcaattctctgttcgtgatttcgcgtttccttcgcaattaaaatatatatcgcaaggatttttcctttccattggcatggcccttcctgccgccctaggtgttggaattgccatgcaagaccactcaaacgctcacatcaatggtggcaacgtaaaagaggactataagccaagattaattttgtttgaaggtgacggtgcagcacagatgacaatccaagaactgagcaccattctgaagtgcaatattccactagaagttatcatttggaacaataacggctacactattgaaagagccatcatgggccctaccaggtcgtataacgacgttatgtcttggaaatggaccaaactatttgaagcattcggagacttcgacggaaagtatactaatagcactctcattcaatgtccctctaaattagcactgaaattggaggagcttaagaattcaaacaaaagaagcgggatagaacttttagaagtcaaattaggcgaattggatttccccgaacagctaaagtgcatggttgaagcagcggcacttaaaagaaataaaaaatag

SEQ ID NO: 10 - DNA, 1917 bp, Artificial Sequence
  optimized ScARO10*
atggctccggttaccatcgaaaaattcgttaaccaggaagaacgtcacctggtttctaaccgttctgctaccatcccgttcggtgaatacatcttcaaacgtctgctgtctatcgacaccaaatctgttttcggtgttccgggtgacttcaacctgtctctgctggaatacctgtactctccgtctgttgaatctgctggtctgcgttgggttggtacctgcaacgaactgaacgctgcttacgctgctgacggttactctcgttactctaacaaaatcggttgcctgatcaccacctacggtgttggtgaactgtctgctctgaacggtatcgctggttctttcgctgaaaacgttaaagttctgcacatcgttggtgttgctaaatctatcgactctcgttcttctaacttctctgaccgtaacctgcaccacctggttccgcagctgcacgactctaacttcaaaggtccgaaccacaaagtttaccacgacatggttaaagaccgtgttgcttgctctgttgcttacctggaagacatcgaaaccgcttgcgaccaggttgacaacgttatccgtgacatctacaaatactctaaaccgggttacatcttcgttccggctgacttcgctgacatgtctgttacctgcgacaacctggttaacgttccgcgtatctctcagcaggactgcatcgtttacccgtctgaaaaccagctgtctgacatcatcaacaaaatcacctcttggatctactcttctaaaaccccggctatcctgggtgacgttttaaccgaccgttacggtgtaagcaacttcctgaacaaactgatctgcaaaaccggtatctggaacttctctaccgttatgggtaaatctgttatcgacgaatctaacccgacctacatgggtcagtacaacggtaaagaaggtctgaaacaggtttacgaacacttcgaactgtgcgacctggttctgcacttcggtgttgacatcaacgaaatcaacaacggtcactacaccttcacctacaaaccgaacgctaaaatcatccagttccacccgaactacatccgtctggttgacacccgtcagggtaacgaacagatgttcaaaggtatcaacttcgctccgatcctgaaagaactgtacaaacgtatcgacgtttctaaactgtctctgcagtacgactctaacgttacccagtacaccaacgaaaccatgcgtctggaagacccgaccaacggtcagtcttctatcatcacccaggttcacctgcagaaaaccatgccgaaattcctgaacccgggtgacgttgttgtttgcgaaaccggttctttccagttctctgttcgtgacttcgctttcccgtctcagctgaaatacatctctcagggtttcttcctgtctatcggtatggctctgccggctgctctgggtgttggtatcgctatgcaggaccactctaacgctcacatcaacggtggtaacgttaaagaagactacaaaccgcgtctgatcctgttcgaaggtgacggtgctgctcagatgaccatccaggaactgtctaccatcctgaaatgcaacatcccgctggaagttatcatctggaacaacaacggttacaccatcgaacgtgctatcatgggtccgacccgttcttacaacgacgttatgtcttggaaatggaccaaactgttcgaagcgttcggtgacttcgacggtaaatacaccaactctaccctgatccagtgcccgtctaaactggctctgaaactggaagaactgaaaaactctaacaaacgttctggtatcgaactgctggaagttaaactgggtgaactggacttcccggaacagctgaaatgcatggttgaagctgctgctctgaaacgtaacaaaaaataaaagctttaa

SEQ ID NO: 11 - DNA, 1122 bp, Escherichia coli
atggttgctgaattgaccgcattacgcgatcaaattgatgaagtcgataaagcgctgctgaatttattagcgaagcgtctggaactggttgctgaagtgggcgaggtgaaaagccgctttggactgcctatttatgttccggagcgcgaggcatctatgttggcctcgcgtcgtgcagaggcggaagctctgggtgtaccgccagatctgattgaggatgttttgcgtcgggtgatgcgtgaatcttactccagtgaaaacgacaaaggatttaaaacactttgtccgtcactgcgtccggtggttatcgtcggcggtggcggtcagatgggacgcctgttcgagaagatgctgaccctctcgggttatcaggtgcggattctggagcaacatgactgggatcgagcggctgatattgttgccgatgccggaatggtgattgttagtgtgccaatccacgttactgagcaagttattggcaaattaccgcctttaccgaaagattgtattctggtcgatctggcatcagtgaaaaatgggccattacaggccatgctggtggcgcatgatggtccggtgctggggctacacccgatgttcggtccggacagcggtagcctggcaaagcaagttgtggtctggtgtgatggacgtaaaccggaagcataccaatggtttctggagcaaattcaggtctggggcgctcggctgcatcgtattagcgccgtcgagcacgatcagaatatggcgtttattcaggcactgcgccactttgctacttttgcttacgggctgcacctggcagaagaaaatgttcagcttgagcaacttctggcgctctcttcgccgatttaccgccttgagctggcgatggtcgggcgactgtttgctcaggatccgcagctttatgccgacatcattatgtcgtcagagcgtaatctggcgttaatcaaacgttactataagcgtttcggcgaggcgattgagttgctggagcagggcgataagcaggcgtttattgacagtttccgcaaggtggagcactggttcggcgattacgcacagcgttttcagagtgaaagccgcgtgttattgcgtcaggcgaatgacaatcgccagtaa

SEQ ID NO: 12 - DNA, 1122 bp, Artificial Sequence
  tyrA∧fbr (M53I and A354V)
atggttgctgaattgaccgcattacgcgatcaaattgatgaagtcgataaagcgctgctgaatttattagcgaagcgtctggaactggttgctgaagtgggcgaggtgaaaagccgctttggactgcctatttatgttccggagcgcgaggcatctatcttggcctcgcgtcgtgcagaggcggaagctctgggtgtaccgccagatctgattgaggatgttttgcgtcgggtgatgcgtgaatcttactccagtgaaaacgacaaaggatttaaaacactttgtccgtcactgcgtccggtggttatcgtcggcggtggcggtcagatgggacgcctgttcgagaagatgctgaccctctcgggttatcaggtgcggattctggagcaacatgactgggatcgagcggctgatattgttgccgatgccggaatggtgattgttagtgtgccaatccacgttactgagcaagttattggcaaattaccgcctttaccgaaagattgtattctggtcgatctggcatcagtgaaaaatgggccattacaggccatgctggtggcgcatgatggtccggtgctggggctacacccgatgttcggtccggacagcggtagcctggcaaagcaagttgtggtctggtgtgatggacgtaaaccggaagcataccaatggtttctggagcaaattcaggtctggggcgctcggctgcatcgtattagcgccgtcgagcacgatcagaatatggcgtttattcaggcactgcgccactttgctacttttgcttacgggctgcacctggcagaagaaaatgttcagcttgagcaacttctggcgctctcttcgccgatttaccgccttgagctggcgatggtcgggcgactgtttgctcaggatccgcagctttatgccgacatcattatgtcgtcagagcgtaatctggcgttaatcaaacgttactataagcgtttcggcgaggcgattgagttgctggagcagggcgataagcaggcgtttattgacagtttccgcaaggtggagcactggttcggcgattacgtacagcgttttcagagtgaaagccgcgtgttattgcgtcaggcgaatgacaatcgccagtaa

SEQ ID NO: 13 - DNA, 18 bp, Artificial Sequence
  primer
ctcgagcaccaccaccac

SEQ ID NO: 14 - DNA, 41 bp, Artificial Sequence
  primer
actttaagaaggagatatacatatggctccggttaccatcg

SEQ ID NO: 15 - DNA, 18 bp, Artificial Sequence
  primer
ctcgagcaccaccaccac

SEQ ID NO: 16 - DNA, 51 bp, Artificial Sequence
  primer
aacaaaattatttctattaggtaccttattttttgttacgtttcagagcag

SEQ ID NO: 17 - DNA, 35 bp, Artificial Sequence
  primer
cccaagcttttattttttgttacgtttcagagcag

SEQ ID NO: 18 - DNA, 45 bp, Artificial Sequence
  primer
gtttaactttataaggaggaaaaaaaatgcaaaaagacgcgctga

SEQ ID NO: 19 - DNA, 22 bp, Artificial Sequence
  primer
cggaagcgttaaatccgaatag

SEQ ID NO: 20 - DNA, 22 bp, Artificial Sequence
  primer
ctattcggatttaacgcttccg

SEQ ID NO: 21 - DNA, 44 bp, Artificial Sequence
  primer
aacaaaattatttctattaggtaccttaagccacgcgagccgtc

SEQ ID NO: 22 - DNA, 40 bp, Artificial Sequence
  primer
gtggcttaagcggcctaatacgactcactataggggaatt

SEQ ID NO: 23 - DNA, 40 bp, Artificial Sequence
  primer
tttctattagcggccgaattcttagtgacggaaatcaatc

SEQ ID NO: 24 - DNA, 44 bp, Artificial Sequence
  primer
aacaaaattatttctattaggtaccggggaattgttatccgctc

SEQ ID NO: 25 - DNA, 48 bp, Artificial Sequence
  primer
ggtacctaatagaaataattttgtttaactttataaggaggaaaaaaa

SEQ ID NO: 26 - DNA, 46 bp, Artificial Sequence
  primer
cagtggtggtggtggtggtgctcgagttactggcgattgtcattcg

SEQ ID NO: 27 - DNA, 26 bp, Artificial Sequence
  primer
cccccatgggatcacagatcatacac

SEQ ID NO: 28 - DNA, 24 bp, Artificial Sequence
  primer
ccggatccttagtcctggcttttc

SEQ ID NO: 29 - DNA, 54 bp, Artificial Sequence
  primer
ttgacaattaatcatccggctcgtataatgggaattgtgagcggataacaattc

SEQ ID NO: 30 - DNA, 53 bp, Artificial Sequence
  primer
cattatacgagccggatgattaattgtcaagcaggagtcgcataagggagagc

SEQ ID NO: 31 - DNA, 18 bp, Artificial Sequence
  Promoter
taatacgactcactatag

SEQ ID NO: 32 - DNA, 31 bp, Artificial Sequence
  promoter
tttacactttatgcttccggctcgtatgttg

SEQ ID NO: 33 - DNA, 29 bp, Artificial Sequence
  promoter
ttgacaattaatcatcggctcgtataatg

SEQ ID NO: 34 - DNA, 30 bp, Artificial Sequence
  promoter
ttgacaattaatcatccggctcgtataatg

SEQ ID NO: 35 - protein, 520 aa, Escherichia coli
MKPEDFRASTQRPFTGEEYLKSLQDGREIYIYGERVKDVTTHPAFRNAAASVAQLYDALHKPEMQDSLCWNTDTGSGGYTHKFFRVAKSADDLRQQRDAIAEWSRLSYGWMGRTPDYKAAFGCALGANPGFYGQFEQNARNWYTRIQETGLYFNHAIVNPPIDRHLPTDKVKDVYIKLEKETDAGIIVSGAKVVATNSALTHYNMIGFGTLEVMGENPDFALMFVAPMDADGVKLISRASYEMVAGATGSPYDYPLSSRFDENDAILVMDNVLIPWENVLIYRDFDRCRRWTMEGGFARMYPLQACVRLAVKLDFITALLKKSLECTGTLEFRGVQADLGEVVAWRNTFWALSDSMCSEATPWVNGAYLPDHAALQTYRVLAPMAYAKIKNIIERNVTSGLIYLPSSARDLNNPQIDQYLAKYVRGSNGMDHVQRIKILKLMWDAIGSEFGGRHELYEINYSGSQDEIRLQCLRQAQNSGNMDKMMAMVDRCLSEYDQDGWTVPHLHNNDDINMLDKLLK

SEQ ID NO: 36 - DNA, 1563 bp, Escherichia coli
atgaaaccagaagatttccgcgccagtacccaacgtcctttcaccggggaagagtatctgaaaagcctgcaggatggtcgcgagatctatatctatggcgagcgagtgaaagacgtcaccactcatccggcatttcgtaatgcggcagcgtctgttgcccagctgtacgacgcactgcacaaaccggagatgcaggactctctgtgttggaacaccgacaccggcagcggcggctatacccataaattcttccgcgtggcgaaaagtgccgacgacctgcgccagcaacgcgacgccatcgctgagtggtcacgcctgagctatggctggatgggccgtaccccagactacaaagccgctttcggttgcgcactgggcgcgaatccgggcttttacggtcagttcgagcagaacgcccgtaactggtacacccgtattcaggaaactggcctctactttaaccacgcgattgttaacccaccgatcgatcgtcatttgccgaccgataaagtgaaagacgtttacatcaagctggaaaaagagactgacgccgggattatcgtcagcggtgcgaaagtggttgccaccaactcggcgctgactcactacaacatgattggcttcggctcggcacaagtgatgggcgaaaacccggacttcgcactgatgttcgttgcgccaatggatgccgatggcgtgaaattaatctcccgcgcctcttatgagatggtcgcgggtgctaccggctcgccatacgactacccgctctccagccgcttcgatgagaacgatgcgattctggtgatggataacgtgctgattccatgggaaaacgtgctgatctaccgcgattttgatcgctgccgtcgctggacgatggaaggcggttttgcccgtatgtatccgctgcaagcctgtgtgcgcctggcagtgaaattagacttcattacggcactgctgaaaaaatcactcgaatgtaccggcaccctggagttccgtggtgtgcaggccgatctcggtgaagtggtagcgtggcgcaacaccttctgggcattgagtgactcgatgtgttcagaagcaacgccgtgggtcaacggggcttatttaccggatcatgccgcactgcaaacctatcgcgtactggcaccaatggcctacgcgaagatcaaaaacattatcgaacgcaacgttaccagtggcctgatctatctcccttccagtgcccgtgacctgaataatccgcagatcgaccagtatctggcgaagtatgtgcgcggttcgaacggtatggatcacgtccagcgcatcaagatcctcaaactgatgtgggatgctattggcagcgaatttggtggtcgtcacgaactgtatgaaatcaactactccggtagccaggatgagattcgcctgcagtgtctgcgccaggcacaaaactccggcaatatggacaagatgatggcgatggttgatcgctgcctgtcggaatacgaccaggacggctggactgtgccgcacctgcacaacaacgacgatatcaacatgctggataagctgctgaaataa

SEQ ID NO: 37 - DNA, 1563 bp, Escherichia coli
atgaaaccagaagatttccgcgccagtacccaacgtcctttcaccggggaagagtatctgaaaagcctgcaggatggtcgcgagatctatatctatggcgagcgagtgaaagacgtcaccactcatccggcatttcgtaatgcggcagcgtctgttgcccagctgtacgacgcactgcacaaaccggagatgcaggactctctgtgttggaacaccgacaccggcagcggcggctatacccataaattcttccgcgtggcgaaaagtgccgacgacctgcgccagcaacgcgacgccatcgctgagtggtcacgcctgagctatggctggatgggccgtaccccagactacaaagccgctttcggttgcgcactgggcgcgaatccgggcttttacggtcagttcgagcagaacgcccgtaactggtacacccgtattcaggaaactggcctctactttaaccacgcgattgttaacccaccgatcgatcgtcatttgccgaccgataaagtgaaagacgtttacatcaagctggaaaaagagactgacgccgggattatcgtcagcggtgcgaaagtggttgccaccaactcggcgctgactcactacaacatgattggcttcggcaccctggaagtgatgggcgaaaacccggacttcgcactgatgttcgttgcgccaatggatgccgatggcgtgaaattaatctcccgcgcctcttatgagatggtcgcgggtgctaccggctcgccatacgactacccgctctccagccgcttcgatgagaacgatgcgattctggtgatggataacgtgctgattccatgggaaaacgtgctgatctaccgcgattttgatcgctgccgtcgctggacgatggaaggcggttttgcccgtatgtatccgctgcaagcctgtgtgcgcctggcagtgaaattagacttcattacggcactgctgaaaaaatcactcgaatgtaccggcaccctggagttccgtggtgtgcaggccgatctcggtgaagtggtagcgtggcgcaacaccttctgggcattgagtgactcgatgtgttcagaagcaacgccgtgggtcaacggggcttatttaccggatcatgccgcactgcaaacctatcgcgtactggcaccaatggcctacgcgaagatcaaaaacattatcgaacgcaacgttaccagtggcctgatctatctcccttccagtgcccgtgacctgaataatccgcagatcgaccagtatctggcgaagtatgtgcgcggttcgaacggtatggatcacgtccagcgcatcaagatcctcaaactgatgtgggatgctattggcagcgaatttggtggtcgtcacgaactgtatgaaatcaactactccggtagccaggatgagattcgcctgcagtgtctgcgccaggcacaaaactccggcaatatggacaagatgatggcgatggttgatcgctgcctgtcggaatacgaccaggacggctggactgtgccgcacctgcacaacaacgacgatatcaacatgctggataagctgctgaaataa

SEQ ID NO: 38 - DNA, 53 bp, Artificial Sequence
  primer
cctctagattaactttaagaaggagtatacatatgaaaccagaagatttccgc

SEQ ID NO: 39 - DNA, 34 bp, Artificial Sequence
  primer
cggcaccctggaagtgatgggcgaaaacccggac

SEQ ID NO: 40 - DNA, 37 bp, Artificial Sequence
  primer
catcacttccagggtgccgaagccaatcatgttgtag

SEQ ID NO: 41 - DNA, 32 bp, Artificial Sequence
  primer
ccggatccttaaatcgcagcttccatttccag